(19) United States
(12) Patent Application Publication
(10) Pub. No.: US 2019/0017067 A1
(43) Pub. Date: Jan. 17, 2019

(54) GLYPHOSATE TOLERANT PLANTS HAVING MODIFIED 5-ENOLPYRUVYLSHIKIMATE-3-PHOSPHATE SYNTHASE GENE REGULATION

(71) Applicants: Regents of the University of Minnesota, Minneapolis, MN (US); Donald Danforth Plant Science Center, St. Louis, MO (US)

(72) Inventors: Aaron W. Hummel, St. Louis, MO (US); Daniel F. Voytas, Falcon Heights, MN (US); Rebecca Bart, St. Louis, MO (US); Nigel Taylor, St. Louis, MO (US); Raj Deepika Chauhan, St. Louis, MO (US)

(21) Appl. No.: 16/069,703

(22) PCT Filed: Jan. 12, 2017
(86) PCT No.: PCT/US2017/013208

§ 371 Date: Jul. 12, 2018

Related U.S. Application Data
(60) Provisional application No. 62/277,734, filed on Jan. 12, 2016.

(51) Int. Cl. C12N 15/82

(52) U.S. Cl. CPC C12N 15/8275; C12N 15/8213

(57) ABSTRACT

This document provides methods and materials related to plants (e.g., nontransgenic plants) that are tolerant to glyphosate-based herbicides. For example, glyphosate tolerant plants can have an altered expression profile (e.g., increased expression levels) of a 5-enolpyruvylshikimate-3-phosphate synthase (EPSPS) gene (e.g., a modified EPSPS gene).

### CLAIM OF PRIORITY

This application claims the benefit of U.S. Patent Application Ser. No. 62/277,734, filed on Jan. 12, 2016. The entire contents of which are hereby incorporated by reference.

### TECHNICAL FIELD

This document relates to methods and materials for generating plants (e.g., nontransgenic plants) that are tolerant to glyphosate-based herbicides. For example, this document provides glyphosate tolerant plants having an altered expression profile (e.g., increased expression levels) of a 5-enolpyruvylshikimate-3-phosphate synthase (EPSPS) gene (e.g., a modified EPSPS gene), as well as methods and materials for making and using glyphosate tolerant plants.

### SEQUENCE LISTING

The instant application includes a sequence listing in electronic format submitted to the United States Patent and Trademark Office via the electronic filing system. The ASCII text file, which is incorporated-by-reference herein, is titled “09531-0356WO1_ST25.txt,” was created on Jan. 12, 2017, has a size of 498 kilobytes.

### BACKGROUND

In 2012, the global value of herbicide resistant crops was estimated at US $18.7 billion (International service for the Acquisition of Agri-Biotech Applications, “Herbicide Tolerance Technology: Glyphosate and Glufosinate,” Pockets of Knowledge 15 Aug. 2015). Since then, agricultural land devoted to biotech crops has continued to increase, reaching a record 181.5 million hectares worldwide in 2014 (International Service for the Acquisition of Agri-Biotech Applications, “Biotech Crops Show Continued Growth, Benefits in 2014, Global Plantings Increase by 6 Million Hectares,” ISAAA Brief 49-2014: Press Release 28 Jan. 2015).

The use of glyphosate for chemical weed control in agricultural operations is one of the biggest advances in modern farming. Glyphosate is the most widely used herbicide in the world. Its popularity is due largely to favorable properties such as high efficacy, low toxicity to animals, little persistence in the environment, flexible application timing, broad spectrum weed control, and low cost. Its use has increased further with the development of ROUNDUP READY® and similar traits in major crops.

### SUMMARY

All glyphosate resistant traits commercialized to date have had to undergo deregulation in the markets in which they are traded. Deregulation presents a substantial barrier in new product development costs and time to market, preventing the development of glyphosate resistance traits in secondary crops. Regulatory approval costs and social acceptance continue to be major worldwide barriers to the development of new genetically modified crops (e.g., crops that carry transgenes such as DNA from a different species).

This document is based, at least in part, on the discovery that an altered expression profile of a 5-enolpyruvylshikimate-3-phosphate synthase (EPSPS) gene (e.g., an EPSPS gene having a modified coding sequence) in plants can confer glyphosate tolerance. Glyphosate resistant crops enable post-emergence weed control, benefiting farmers with increased flexibility and efficiency. This document also is based, at least in part, on the discovery that gene editing techniques can be used to produce nontransgenic plants having an altered expression profile of one or more EPSPS genes. Nontransgenic strategies, such as gene editing, can be used to produce glyphosate tolerant plants (e.g., in secondary crops and additional plant varieties) with less cost associated with regulatory approval, as well as increased public confidence in the safety of the products.

Thus, this document provides methods and materials related to plants (e.g., nontransgenic plants) that are tolerant to glyphosate-based herbicides. For example, this document provides glyphosate tolerant plants having an altered expression profile (e.g., increased expression levels) of a 5-enolpyruvylshikimate-3-phosphate synthase (EPSPS) gene (e.g., an EPSPS gene having a modified coding sequence), as well as methods and materials for making and using glyphosate tolerant plants having an altered expression profile of the modified EPSPS gene.

In some aspects, this document provides methods for generating a glyphosate tolerant plant. The methods can include site-specific editing of a plant genome in order to modify EPSPS gene regulation, such that a modification to EPSPS gene regulation is effective to cause an altered EPSPS gene expression profile compared to an EPSPS gene expression profile of a non-edited plant genome, and such that the altered EPSPS expression profile confers glyphosate tolerance. The site-specific editing can include operably linking an EPSPS gene to an alternative promoter (e.g., an actin promoter, a ubiquitin promoter, a promoter that drives expression of Manes.17G101400 gene, a promoter that drives expression of Manes.09G138100, or a promoter that drives expression of Manes.11G090400) at a native genomic location of the alternative promoter. The alternative promoter can be a constitutive promoter, and the altered EPSPS gene expression profile can include increased EPSPS expression. The alternative promoter can be a tissue-specific promoter (e.g., a meristem specific promoter such as CLV3, FIL, and WUS), and the altered EPSPS gene expression profile can include a change in spatial expression of the EPSPS gene (e.g., increased EPSPS gene expression in a meristem). The site-specific editing can include operably linking an EPSPS gene to a recombinant promoter at a native genomic location of the EPSPS gene. A recombinant promoter can be a constitutive promoter and the altered EPSPS gene expression profile can be increased EPSPS expression. A recombinant promoter can be a tissue-specific promoter (e.g., a meristem-specific promoter such as CLV3, FIL, or WUS) and the altered EPSPS gene expression profile can be a change in spatial expression of the EPSPS gene (e.g., increased expression in a meristem). A recombinant promoter can be an inducible promoter (e.g., Es or PR-1). The site-specific editing can include introducing an enhancer (e.g., a 35s enhancer, a translational enhancer from the tobacco etch virus, or a pea PetE enhancer) into an endogenous EPSPS regulatory sequence and the altered EPSPS gene expression profile can be an increase in EPSPS expression. The EPSPS gene can include a modified EPSPS coding sequence. A modified EPSPS coding sequence can include a substitution of the glycine at amino acid 101 (G101), a substitution of the threonine at amino acid 102 (T102), a substitution of the proline at amino acid 106 (P106), a substitution of the glycine at amino acid 144 (G144), a substitution of the alanine at amino acid 192 (A192), or any combination thereof. For example, a modified EPSPS coding sequence can include a T102I substitution and a P106A substitution, a G101A substitution and an A192T substitution, a T102I substitution and a P106C substitution, a T102I substitution and a P106I substitution, a T102I substitution and a P106S substitution, a G101A substitution and a G144N substitution, or a G101A substitution and a G144D substitution.

In some aspects, this document provides methods for generating a glyphosate tolerant plant (e.g., nontransgenic glyphosate tolerant plant). Such methods can include introducing into a plant cell a site-specific nuclease and a repair template including an EPSPS gene or gene fragment, selecting a plant cell having an altered expression profile of the EPSPS gene or gene fragment compared to an EPSPS gene expression profile of a non-edited plant genome, wherein the altered expression profile of said modified EPSPS gene is effective to confer glyphosate tolerance, and regenerating a glyphosate tolerant plant from the selected plant cell. A site-specific nuclease can be a zinc finger nuclease (ZFN), a transcription activator-like effector nuclease (TALE nuclease), a CRISPR-associated nuclease (e.g., Cas9 or Cpf1), or a homing endonuclease (HE). The repair template further can include a constitutive promoter. The constitutive promoter can be from the same species as the EPSPS gene or gene fragment. Both the constitutive promoter and the EPSPS gene or gene fragment can be from the same species as the plant cell. The EPSPS gene or gene fragment can include a modified EPSPS coding sequence. The modified EPSPS coding sequence can include a T102I substitution and a P106A substitution, a G101A substitution and an A192T substitution, a T102I substitution and a P106C substitution, a T102I substitution and a P106I substitution, a T102I substitution and a P106S substitution, a G101A substitution and a G144N substitution, or a G101A substitution and a G144D substitution.

In some aspects, this document provides glyphosate tolerant plants (e.g., nontransgenic glyphosate tolerant plants). Such plants can include a genome edited in a site-specific manner to modify EPSPS gene regulation, such that the modified EPSPS gene regulation is effective to cause an altered expression profile of an EPSPS gene compared to an EPSPS gene expression profile of a non-edited plant genome, such that the altered expression profile confers glyphosate tolerance to the plants. The modification to EPSPS gene regulation can include introducing an enhancer (e.g., a 35s enhancer, a translational enhancer from the tobacco etch virus, or a pea PetE enhancer) into an EPSPS gene regulatory sequence and the altered EPSPS gene expression profile can be an increase in EPSPS gene expression. The enhancer can be introduced into an EPSPS promoter, into an EPSPS intron, upstream of an EPSPS promoter, or downstream of an EPSPS terminator. The edited genome can include an EPSPS gene operably linked to an alternative promoter (e.g., an actin promoter, a ubiquitin promoter, a promoter that drives expression of Manes.17G101400 gene, a promoter that drives expression of Manes.09G138100, or a promoter that drives expression of Manes.11G090400) at a native genomic location of the alternative promoter and the altered EPSPS gene expression profile can be increased EPSPS expression. The edited genome can include an EPSPS gene operably linked to a recombinant promoter at a native genomic location of the EPSPS gene. The recombinant promoter can be a constitutive promoter and the altered EPSPS gene expression profile can be increased EPSPS expression. The recombinant promoter can be a tissue-specific promoter (e.g., a meristem-specific promoter such as CLV3, FIL, or WUS) and the altered EPSPS gene expression profile is a change in spatial expression of the EPSPS gene (e.g., expression in a meristem). The recombinant promoter can be an inducible promoter (e.g., Es or PR-1). The glyphosate tolerant plant can be a monocotyledonous plant (e.g., maize, rice, wheat, barley, sugarcane, oat, rye, millet, sorghum, switchgrass, turfgrass, or bamboo). The glyphosate tolerant plant can be a dicotyledonous plant (e.g., bean, soybean, cotton, pea, cowpea, peanut, almond, walnut, apple, plum, peach, pear, citrus, sugar beet, squash, melon, cassava, tomato, pepper, canola, banana, flax, or sunflower). The EPSPS gene can include a modified EPSPS coding sequence. The modified EPSPS coding sequence can include a G101 substitution, a T102 substitution, a P106 substitution, a G144 substitution, a A192 substitution, or any combination thereof. The modified EPSPS coding sequence can include a T102I substitution and a P106A substitution, a G101A substitution and an A192T substitution, a T102I substitution and a P106C substitution, a T102I substitution and a P106I substitution, a T102I substitution and a P106S substitution, a G101A substitution and a G144N substitution, or a G101A substitution and a G144D substitution.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph showing quantitative PCR data. The EPSPS gene is expressed at a relatively low level in meristems compared to other tissues. Values are reported relative to expression in leaf tissue. FEC=friable, embryogenic callus.

FIG. 2 shows schematics of gene models used to test the levels of glyphosate resistance conferred by expression of an EPSPS gene. Expression was driven by either a cassava TME7 native EPSPS promoter or a tandemly repeated cauliflower mosaic virus 35s strong constitutive (double 35s) promoter. The EPSPS coding sequence was either wild type (WT) or modified to contain double amino acid substitutions that confer reduced inhibition by glyphosate.

FIG. 3 shows semi-quantitative PCR to assess expression of the introduced EPSPS gene models in leaves of regenerated plants. H001: WT EPSPS with native promoter; H002: T102I/P106A (TIPA) EPSPS with native promoter; H003: WT EPSPS with 2×35s promoter; H004: T201I/P106A EPSPS with 2×35s promoter; H009: G101A/A192T EPSPS with native promoter; H010: G101A/A192T EPSPS with 2×35s promoter; H013: T102I/P106I EPSPS with native promoter; H014: T102I/P106I EPSPS with 2×35s promoter. Expression was compared to that of the reference gene GTPb. GTPb (-RT) was used as a negative control reaction.

FIGS. 4A-B show an in vitro rooting test to compare glyphosate resistance in transgenic plant lines in the presence of glyphosate. FIG. 4A is a graph showing the average number of roots on stem cuttings derived from five independent transgenic events with each gene model vector. FIG. 4B is a graph showing the average root length on stem cuttings derived from five independent transgenic events. The roots were counted and measured after two weeks of growth on media containing 0.05 mM glyphosate.

FIGS. 5A-H show results of de novo gene model transformation events selected on media supplemented with glyphosate. FIG. 5A is an image of a transformed embryogenic callus on glyphosate-containing media 5 weeks after co-cultivation. FIG. 5B is an image of the maturation of embryogenic callus to form green cotyledon stage embryos from a highly expressing line. FIG. 5C is an image of the inhibition of growth of a low expressing callus line on glyphosate containing media. FIG. 5D is an image of a germinating embryo on glyphosate free media. FIG. 5E is an image of a rooted transgenic plantlet on glyphosate free media. FIG. 5F shows a series of images illustrating key points in the process of gene model transformation, selection with glyphosate, and testing for herbicide tolerance. FIG. 5G shows a table summarizing the of de novo gene model transformation events recovered under glyphosate selection and compared to paromomycin selection. Only one event was recovered with the EPSPS endogenous promoter under glyphosate selection, whereas 29 events were recovered with the double 35s promoter under glyphosate selection. This indicates the superiority of highly expressed EPSPS variants for robust herbicide tolerance during regeneration compared to expression levels provided by the native EPSPS promoter. The highly expressed EPSPS variants could also be used in a selectable marker cassette in a plant transformation program. FIG. 5H shows an example of the more vigorous rooting and vegetative growth from plants containing the double 35s promoter driving the TIPA enzyme compared to plant containing the endogenous EPSPS promoter driving the TIPA enzyme in the presence of glyphosate. In each image, the top left plate contains WT control plants on media without glyphosate and the bottom left plate contains WT control plants on media containing glyphosate. All other plates contain plants with the respective gene model on media containing glyphosate.

FIGS. 6A-C shows the response of cassava plants transformed with the EPSPS gene models after application of glyphosate. FIG. 6A is a graph showing damage to plants transgenic for gene construct H002 (EPSPS promoter driving the TIPA EPSPS sequence) and H004 (double 35S promoter driving the TIPA EPSPS sequence) over time. Impact of herbicide application was assessed three times weekly on a scale of 1-7 for damage to the shoots where 1=no damage to 7=plant death. Average damage shown by H004 plants was less than 4 while damage to H002 plants was about 6. FIG. 6B is a graph showing damage to plants transgenic for gene construct H009 (EPSPS promoter driving the GAAT (G101A/A192T) EPSPS sequence) and H010 (double 35S promoter driving the GAAT EPSPS sequence) in the same assay. Average damage shown by H010 plants was 3-4 while damage to H009 plants was 4-6.5. FIG. 6C is a graph showing damage to plants transgenic for gene construct H013 (EPSPS promoter driving the TIPI (T102I/P106I) EPSPS sequence) and H014 (double 35S promoter driving the TIPI EPSPS sequence) in the same assay. Average damage shown by H014 plants was 1-3.5, while damage to H013 plants was 4-5.5. A small number of H013 and H014 lines showed no resistance to glyphosate. This is likely due to no or poor expression of the gene models in these events, which were not characterized before challenge with glyphosate. Together these data show the superior herbicide tolerance of EPSPS enzymes expressed at high levels compared to expression from the endogenous promoter, regardless of which combination of amino acid substitutions is used.

FIGS. 7A-C show example phenotypes of herbicide tolerance by plants containing either the H004 or the H010 gene models. FIG. 7A is a photo showing representative response of plant lines transgenic for H004 21 days after application of 50 mg active ingredient per plant. WT Control—surfactant only, WT—non-transgenic plant, 018, 019, 020—plants from three independent events of H004 transformation. FIG. 7B is an example photo showing root health of a plant derived from event 019 with the H004 gene model after application of 50 mg active ingredient per plant. Storage root development is comparable to the H004 019 plant treated with surfactant only. WT Control—WT plant that was not treated, —Surfactant only—H004 019 plant sprayed only with surfactant, Sprayed—H004 019 plant treated with surfactant and 50 mg active ingredient. FIG. 7C is a photo showing representative response of plant lines transgenic for H010 21 days after application of 50 mg active ingredient per plant. WT Control—surfactant only, WT—non-transgenic plant, 016, 017, 018—plants from three independent events of H010 transformation.

FIGS. 8A-D show the identification and testing of native plant promoters suitable for driving EPSPS expression. FIG. 8A shows cassava genes with constitutive expression profiles identified from an RNA-seq experiment designed to assess global gene expression in different tissues. FIG. 8B shows comparative activity of the β-Glucuronidase (GUS) enzyme expressed by several of these promoters from T-DNAs in transgenic cassava leaves. FIG. 8C shows eGFP expression driven by a double cauliflower mosaic virus 35S promoter (2×35s) and cassava promoters with functional annotations of cold, circadian rhythm, and RNA binding 1 promoter (RB1); dehydrin family promoter (DFP); and rotamase CYP 1 promoter (RC1). Based on intensity of eGFP fluorescence, each promoter drove eGFP expression to a level near to that of the double 35s promoter. FIG. 8D shows GUS expression driven by 1200, 1000, and 526 nucleotide lengths of the RB1 promoter transiently delivered on T-DNAs via Agroinfiltration of tobacco leaves. The left side of each leaf was infiltrated by a T-DNA expressing GUS driven by the double 35s promoter and the right side of each leaf was infiltrated by a T-DNA expressing GUS driven by the RB1 promoter fragment or no promoter. This indicates that fragments of the RB1 promoter as short as 526 nucleotides are sufficient to drive expression of the GUS gene to similar levels as the double 35s promoter.

FIG. 9A-B show the response of cassava plants transformed with TIPA gene models, expressed by the RB1 and DFP promoters identified in FIG. 8, after application of glyphosate. FIG. 9A is a graph showing damage to plants transgenic for gene construct H063 (RB1 promoter driving the TIPA EPSPS sequence) over time. The assay was performed as described for FIG. 6. Average damage shown by two independent lines was 3-4, which is comparable to the performance of the H004 gene model (double 35s promoter driving the TIPA EPSPS sequence) shown in FIG. 6. One H063 line showed no resistance to glyphosate. This is likely due to no or poor expression of the gene model in this event, which was not characterized before challenge with glyphosate. FIG. 9B is a graph showing damage to plants transgenic for gene construct H064 (DFP promoter driving the TIPA EPSPS sequence) in the same assay. Average damage shown by four independent lines was around 4, which is comparable to the performance of the H004 gene model (double 35s promoter driving the TIPA EPSPS sequence) shown in FIG. 6. One H064 line showed no resistance to glyphosate. This is likely due to no or poor expression of the gene model in this event, which was not characterized before challenge with glyphosate. Together these data show that robust glyphosate tolerance can be achieved by driving EPSPS enzymes to high levels of expression with properly selected plant-derived promoters.

FIGS. 10A-E show EPSPS gene editing with a strong constitutive promoter in cassava protoplasts. FIGS. 10A-C show schematics of an approach to perform gene editing of the EPSPS gene. After introduction of gene editing reagents into the plant cell, Cas9-based SSNs produce DSBs in the genomic target and, in the case of the NHEJ knockin strategy, in the equivalent locations of the repair template (RT) (A). The RT fragment is incorporated into the broken chromosome by NHEJ or HR, or some combination of the two pathways (B) to produce an edited EPSPS allele with amino acid substitutions driven by a strong constitutive promoter (C). FIG. 10D shows a schematic for a PCR detection strategy of gene editing events in protoplast populations treated with editing reagents. By pairing one primer that binds only in the genome with another that binds only in the repair template (e.g., primer 1 paired with primer 2, or primer 3 paired with primer 4), it is possible to selectively amplify the left- or right-junctions of gene editing events out of a population consisting mostly of unmodified genomes. FIG. 10E shows PCR amplicons obtained from the left- and right-junction PCRs following the strategy described. The expected bands for gene editing events at both junctions are indicated in samples treated with a vector having a RT using either the NHEJ or HR repair pathways (SEQ ID NO: 9) or a vector having a RT using only the HR repair pathway (SEQ ID NO: 10) as indicated.

FIGS. 11A-B show sequences obtained from Sanger-sequencing of clones derived from the gene editing events according to the shown in FIG. 10. FIG. 11A shows sequences at the left-junction of the gene editing event. FIG. 11B shows sequences at the right-junction of the gene editing event.

FIG. 12 shows schematics of gene editing models. An EPSPS gene is edited to contain T102I and P106A substitutions and is edited to have expression driven by a double 35s promoter, or as nonlimiting examples of plant promoters with strong constitutive expression profiles, by cassava promoters from genes with functional annotations of cold, circadian rhythm, and RB1; DFP; or RC1.

FIGS. 13A-F show the generation, recovery, and verification of plants with 2×35s promoter TIPA gene editing events by selection on media supplemented with 2.5 to 5.0 mM glyphosate or with 45 uM paromomycin. FIG. 13A shows an EPSPS gene-edited embryogenic callus growing on glyphosate-containing media 5 weeks after co-cultivation with Agrobacterium. FIG. 13B shows formation of green cotyledon stage embryos on glyphosate-containing media. FIG. 13C shows germinating embryos of putative EPSPS gene editing events intended to introduce the TIPA EPSPS configuration driven by the double 35S promoter. The table given in FIG. 13D shows the frequency of glyphosate or paromomycin resistant event recovery after treatment with each of several repair templates and selection strategies. FIG. 13E shows PCR-detection of EPSPS editing events by amplifying across the entire site of the gene editing event (full length) or by amplifying across the left homology arm from the genome into the double 35s promoter (left junction). The table indicates what is shown in each lane of the gel images. FIG. 13F shows a schematic of a successful editing event and the primers used for PCR validations in FIG. 13E.

FIG. 14 shows Sanger sequencing confirmation of gene editing events. Panels A and B are data from a glyphosate tolerant plant generated by NHEJ using vector H055. In Panel A, the top sequence depicts the expected gene targeting product (SEQ ID NO:31). Below this figure are Sanger sequencing reads for H0551A-139-VLP139_C09.ab1 (SEQ ID NO:32) and H0551A-140-VLP140_D09.ab1 (SEQ ID NO:33) and their consensus sequence (SEQ ID NO:34) showing that the TIPA mutations were incorporated. Panel B shows the site of Cas9 cleavage within the target sequence (SEQ ID NO:35). Below this figure are Sanger sequencing reads for H0551A-140-VLP140 D09.ab1 (SEQ ID NO:36), H0551A-141-VLP141_E09.ab1 (SEQ ID NO:37), and H0551A-476-VLP476_A09.ab1, SEQ ID NO:38) and their consensus sequence (SEQ ID NO:39). Note that mutations have been introduced due to imprecise repair by NHEJ. Panels C and D illustrate DNA sequences from a glyphosate tolerant plant generated by HR using vector H056. As in Panel A, the TIPA mutations have been incorporated into the chromosome. In Panel C, top sequence depicts the expected gene targeting product (SEQ ID NO:40). Below this figure are Sanger sequencing reads for H0561E-139-VLP139_DH10.ab1 (SEQ ID NO:41), H0561E-140-VLP140_A11.ab1 (SEQ ID NO:42) and H0561E-529-VLP529_G10.ab1 (SEQ ID NO:43) and their consensus sequence (SEQ ID NO:44). Panel D shows that the site of Cas9 cleavage lacks mutations, indicative of precise repair by HR. The top sequence is the target sequence (SEQ ID NO:45). Below this figure are Sanger sequencing reads for H0561E-140-VLP140_A11.ab1 (SEQ ID NO:46), H0561E-529-VLP529_G10.ab1 (SEQ ID NO:47), and H0561E-141-VLP141_B11.ab1, SEQ ID NO:48) and their consensus sequence (SEQ ID NO:49).

FIG. 15A-C show the response of cassava plants edited to contain the double 35s promoter driving the TIPA enzyme after application of glyphosate. FIG. 15A is a graph showing damage to edited plants over time after treatment with glyphosate. The glyphosate tolerance is equivalent to performance of the best H004 (double 35S promoter driving the TIPA EPSPS sequence) event, which is shown in FIG. 15B. The assay was performed as described for FIG. 6. FIG. 15C shows a representative phenotype of edited plants 21 days after application of glyphosate at a rate 50 mg active ingredient per plant. WT Control—WT plant treated with surfactant only, WT treated—WT plant treated with glyphosate, H004 019 treated—plant from the 019 event with the H004 gene model treated with glyphosate, H056 001 treated—plant from the 001 event edited with the H056 vector (which produces an event with the double 35s promoter driving the TIPA enzyme) treated with glyphosate. Together these data show the generation of robust glyphosate tolerance by the editing of a strong constitutive promoter together with the TIPA amino acid substitutions into a native plant EPSPS allele.

### DETAILED DESCRIPTION

This document relates to plants (e.g., nontransgenic plants) that are tolerant to glyphosate-based herbicides. For example, this document provides glyphosate tolerant plants having an altered expression profile of an EPSPS gene (e.g., a modified EPSPS gene). In some cases, a glyphosate tolerant plant having an altered expression profile of an EPSPS gene can have increased expression of an EPSPS gene. This document also relates to methods and materials for making and using glyphosate tolerant plants having an altered expression profile of an EPSPS gene. As described herein, gene editing techniques can be used to produce a plant having an altered expression profile of one or more EPSPS genes. For example, one or more modifications can be made to an EPSPS gene regulatory sequence in a plant, which can be effective to cause an altered expression profile of one or more EPSPS genes and confer glyphosate tolerance to the plant.

A glyphosate tolerant plant having an altered expression profile of an EPSPS gene (e.g., a modified EPSPS gene) as described herein can be a transgenic plant or a nontransgenic plant. The term “nontransgenic” plant as used herein refers to a plant which can have one or more modifications made by, for example, gene editing, but which does not contain genetic material from another species stably integrated into the plant genome. Nontransgenic plants and some transgenic plants may achieve nonregulated status under regulatory systems with product-based assessments (e.g., the U.S. regulatory systems). Nontransgenic plants can be made using gene editing strategies as described herein, and can include, for example, site-specific editing of a regulatory sequence and/or a coding sequence, and/or a genomic rearrangement to place a gene under the control of a promoter different from the one by which it is normally controlled.

The term “glyphosate tolerant” as used herein refers to the ability of a plant to survive and/or grow in the presence of one or more glyphosate-based herbicides without exhibiting death, developmental setback, substantial physiological or physical deterioration, or any symptom of herbicide injury. A glyphosate tolerant plant may also be referred to herein as a glyphosate resistant plant.

The term “altered expression profile” as used herein refers to a change in the level of expression (e.g., an increase or decrease), a change in temporal expression as it relates to plant development, and/or a change in spatial expression of the EPSPS gene and/or the EPSPS enzymes encoded by the EPSPS genes when compared to the expression profile observed in an unmodified plant.

Glyphosate tolerant “plants” having an altered expression profile of an EPSPS gene (e.g., an EPSPS gene having a modified coding sequence) as described herein refers to whole plants or plant parts such as plant organs, plant organelles (e.g., plastids), plant tissues, plant propagules, seeds, and plant cells, as well as progeny of the same. Plant cells include, without limitation, cells from seeds, suspension cultures, embryos, meristematic regions, callus tissue, leaves, roots, shoots, gametophytes, sporophytes, pollen, and microspores.

The glyphosate tolerant plants described herein can be derived from any species of plant that is susceptible to glyphosate. In some cases, a plant can be a monocotyledonous plant. In some cases, a plant can be a dicotyledonous plant.

In some cases, a glyphosate tolerant plant as described herein can be a crop plant. Crop plants can include, for example, food crops for human consumption, feed crops for livestock consumption, fiber crops for cordage and textiles, oil crops for consumption or industrial uses, energy crops used to make biofuels, and industrial crops. Non-limiting examples of monocotyledonous crops include maize, rice, wheat, barley, sugarcane, oat, rye, millet, sorghum, switchgrass, turfgrass, and bamboo. Non-limiting examples of dicotyledonous crops include bean, soybean, cotton, pea, cowpea, peanut, almond, walnut, apple, plum, peach, pear, citrus, poplar, pine, sugar beet, squash, melon, strawberry, blueberry, raspberry, cassava, tomato, pepper, canola, banana, flax, and sunflower. In some cases, a glyphosate tolerant plant having an altered expression profile of an EPSPS gene (e.g., a modified EPSPS gene) described herein can be an ornamental plant. Ornamental plants can include, for example, plants grown for decorative purposes in gardens and landscape design projects, as houseplants, for cut flowers, and for specimen display.

A glyphosate tolerant plant having an altered expression profile of an EPSPS gene (e.g., a modified EPSPS gene) as described herein can have increased expression levels of the modified EPSPS gene in one or more tissues.

An altered expression profile of an EPSPS gene can be achieved by modifying a sequence that regulates EPSPS gene expression. In some cases, site-specific gene editing can be used to modify an EPSPS gene regulatory sequence. In some cases, site-specific editing can be used to cause a rearrangement of the plant genome to place an EPSPS gene under the control of an alternative promoter. The one or more modifications to EPSPS gene regulation can be effective to alter the balance of expression (e.g., such that EPSPS is expressed in different tissues of the plant) relative to an unmodified EPSPS gene regulation. The altered expression profile of one or more EPSPS genes can be effective to confer glyphosate tolerance to the plant. Examples of regulatory regions that can be modified or added include, without limitation, promoters, enhancers, suppressors, terminators, 5′- and 3′-untranslated regions, intronic sequences, translation initiation sites, ribosome binding sites, and transcription factor recognition sites. Regulatory regions can be cis- or trans-regulatory regions, and can affect the primary structure, the secondary structure, and/or the stability of the RNA transcript. A promoter can be a constitutive promoter or a regulated promoter. A constitutive promoter may also be referred to as a ubiquitous promoter and can drive transcription of an operably linked nucleic acid molecule (e.g., an EPSPS gene) in most cell types at most times. A regulated promoter may also be referred to as a restricted promoter and can drive transcription of an operably linked nucleic acid molecule (e.g., an EPSPS gene) in response to specific stimuli. A promoter can be a minimal promoter or a composite promoter. A minimal promoter is a promoter having a single genomic promoter fragment derived from a single gene, while a composite promoter is an engineered promoter and can be a synthetic promoter containing a combination of elements from different promoters and/or different origins.

In some cases, a glyphosate tolerant plant having modified EPSPS gene regulation described herein can include one or more modifications to a native promoter region of an EPSPS gene. For example, a glyphosate tolerant plant described herein can include an EPSPS gene having one or more enhancers introduced in any appropriate location such that expression of the EPSPS gene is increased. An enhancer can be a transcriptional enhancer or a translational enhancer. Non-limiting examples of enhancers include the 35s enhancer, the translational enhancer from the tobacco etch virus, and the pea PetE enhancer. Appropriate locations into which an enhancer can be introduced include, for example, an EPSPS promoter, an EPSPS intron, upstream of an EPSPS promoter, downstream of an EPSPS terminator, and/or at another location that enables cis- or trans-activation of the EPSPS gene expression to confer glyphosate resistance.

In some cases, a glyphosate tolerant plant having modified EPSPS gene regulation as described herein can include an EPSPS gene that is under the control of an alternative endogenous promoter. An “alternative” promoter as used herein is a promoter that is native to the plant being modified, but that does not normally control expression of an EPSPS gene. In some cases, an EPSPS gene can be inserted into a plant genome at the native genomic location of the alternative promoter. Alternatively, a genomic rearrangement can result in an EPSPS gene becoming operably linked to an alternative promoter at the native genomic location of the alternative promoter. An alternative promoter can be a constitutive promoter or a regulated promoter. Non-limiting examples of alternative constitutive promoters that can be used to drive expression of a modified EPSPS gene are actin family promoters, ubiquitin family promoters, and promoters driving expression of housekeeping genes. Additional examples of alternative promoters include promoters driving expression of genes in the version 6.1 draft assembly of Manihot esculenta AM560-2 provided on Phytozome 10.3 including, for example, promoters driving expression of genes with IDs of Manes.17G101400, Manes.09G138100, and Manes.11G090400. Additional alternative promoters that can be used to drive expression of a modified EPSPS gene as described herein can be identified using, for example, tissue-specific RNA-seq experiments as described in Example 7.

In some cases, a glyphosate tolerant plant having modified EPSPS gene regulation described herein can include a modified EPSPS gene under the control of a recombinant promoter. A “recombinant promoter” as used herein refers to any promoter that does not natively drive expression of an EPSPS gene. For example, a native EPSPS promoter can be replaced by a recombinant promoter at the native genomic location of the endogenous EPSPS gene. The choice of promoters to be included depends upon several factors, including, but not limited to, efficiency, selectability, inducibility, desired expression level, and desired cell or tissue specificity or non-specificity. For example, expressing an EPSPS gene in plant tissues sensitive to glyphosate can be effective to confer glyphosate tolerance to a plant.

A recombinant promoter can be derived from the same species as the modified EPSPS gene (also referred to as a homologous promoter) or from a different species (e.g., a different plant species, or from any eukaryotic or prokaryotic organism) relative to the modified EPSPS gene (also referred to as a heterologous promoter).

In some cases, a glyphosate tolerant plant having modified EPSPS gene regulation described herein can be made by introducing a constitutive promoter to the native genomic location of an EPSPS gene, such that the constitutive promoter is operably linked to the EPSPS gene. A non-limiting example of a constitutive promoter that can be operably linked to an EPSPS gene is a cauliflower mosaic virus (CaMV) 35S promoter. For example, a glyphosate tolerant plant as described herein can include an EPSPS gene under the control of a constitutive double CaMV 35S promoter.

In some cases, a glyphosate tolerant plant having modified EPSPS gene regulation as described herein can be made by introducing a regulated promoter to the native genomic location of an EPSPS gene, such that the regulated promoter is operably linked to the EPSPS gene. Examples of regulated promoters include, without limitation, cell and/or tissue specific promoters (e.g., promoters that drive transcription predominantly, but not necessarily exclusively, in one cell type or one tissue type), developmentally specific promoters (e.g., promoters that drive transcription based on developmental events), and inducible promoters (e.g., promoters that drive transcription in response to presence of a specific stimulus). It should be understood that some promoters may belong to more than one category of promoter. For example, a promoter that drives transcription in floral meristems can be considered as both a tissue-specific promoter and a developmentally-specific promoter. Non-limiting examples of cell specific and/or tissue specific promoters include promoters that can drive transcription of an operably linked nucleic acid molecule (e.g., an EPSPS gene) in leaf blade, leaf midvein, petiole, stem, lateral bud, shoot apical meristem (SAM), root apical meristem (RAM), immature pollen and other reproductive tissue types, fibrous roots, storage root, friable embryogenic callus (FEC), and/or organized embryogenic structures (OES) of a plant.

Without being bound by theory, it is believed that an altered EPSPS expression profile that changes tissue-specific expression of the EPSPS gene can further optimize glyphosate resistance and plant fitness by maximizing expression in the tissues where glyphosate is most toxic, such as in the meristems. In some embodiments, a glyphosate tolerant plant having an altered expression profile of an EPSPS gene as described herein can include an EPSPS gene under the control of a regulated promoter that can drive transcription of an operably linked nucleic acid molecule (e.g., an EPSPS gene) in meristems (e.g., SAM and/or RAM) of the plant. Examples of regulated promoters that can drive transcription of a modified EPSPS gene in meristems include, without limitation, the Arabidopsis CLV3, FIL, and WUS.

Inducible promoters drive transcription of an operably linked nucleic acid molecule in response to the presence of exogenous conditions or stimuli that can, in some embodiments, be artificially controlled. For example, inducible promoters can be regulated by chemical compounds (e.g., nitrogen, tetracycline, steroids, ethanol, jasmonate, salicylic acid, safeners, gibberellic acid and/or ethylene) or by environmental signals (e.g., light, heat, cold, stress, flooding, drought, phytohormones, and/or wounding). In some cases, an inducible promoter can be responsive to a chemical compound that can be applied to a crop in a field. Examples of regulated promoters that can drive transcription of a modified EPSPS gene in response to specific stimuli include, without limitation, Es (which drives transcription in response to estradiol), and PR-la (which drives transcription in response to salicylic acid, 2,6, dichloroiso-nicotinic acid, and in response to 1,2,3-benzothiadiazole-7-carbothioic acid S-methyl ester).

An EPSPS gene as described herein can be a plant EPSPS gene. An “EPSPS gene” refers to any nucleic acid sequence which can encode an EPSPS enzyme and can include any EPSPS gene or gene fragment. Non-limiting examples of plant EPSPS genes include EPSPS genes from Arabidopsis, maize, rice, wheat, barley, sugarcane, oat, rye, millet, sorghum, switchgrass, turfgrass, bamboo, bean, soybean, cotton, pea, cowpea, peanut, almond, walnut, apple, plum, peach, pear, citrus, poplar, pine, sugar beet, squash, melon, strawberry, blueberry, raspberry, cassava, tomato, pepper, canola, banana, flax, and sunflower. Exemplary EPSPS plant genes are corn EPSPS gene (see, e.g., locus GRMZM5G877500 in the maize genetics and genomics database (MaizeGDB); SEQ ID NO:21), a cassava EPSPS gene (see, e.g., locus cassava4.1_005539 m.g in the Phytozome database; SEQ ID NO:23), a rice EPSPS gene (see, e.g., locus LOC_Os06g04280 in the Plant Expression Database (PLEXdb); SEQ ID NO:25), an Arabidopsis EPSPS gene (see, e.g., locus AT2G45300 in the Arabidopsis Information Resource (TAIR) database; SEQ ID NO:27), and a petunia EPSPS gene (see, e.g., Accession M21084 J03227 (version M21084.1, GI:169190) in the National Center for Biotechnology Information (NCBI) database; SEQ ID NO:29). In some cases, an EPSPS gene is a cassava EPSPS gene.

Exemplary EPSPS enzymes encoded by EPSPS genes include, for example, a corn EPSPS enzyme (SEQ ID NO:22), a cassava EPSPS enzyme (SEQ ID NO:24), a rice EPSPS enzyme (SEQ ID NO:26), an Arabidopsis EPSPS enzyme (SEQ ID NO:28), and a petunia EPSPS enzyme (SEQ ID NO:30).

In some embodiments, a glyphosate tolerant plant having an altered expression profile of an EPSPS gene as described herein can include one or more modifications in the coding sequence of an EPSPS gene. In some embodiments, a “modified EPSPS coding sequence” as described herein can encode an EPSPS enzyme which is less sensitive to glyphosate. A modified EPSPS coding sequence can be present in a single copy or in multiple copies. In some cases, a glyphosate tolerant plant described herein can include a modified EPSPS gene encoding an EPSPS polypeptide with at least one amino acid substitution. Examples of suitable substitutions include those described elsewhere (see, e.g., Yu et al., 2015 Plant Physiol. 167:1440-1447; Sammons et al., 2014 Pest Manag. Sci. 70:1367-1377; WO 2004/074443, and WO 2007/084294). For example, a modified EPSPS gene can include a G101 substitution, a T102 substitution, a P106 substitution, a G144 substitution, a A192 substitution, or any combination thereof. The glycine at amino acid 101 can be substituted with, for example, alanine, such that the modified EPSPS polypeptide includes a G101A substitution. The threonine at amino acid 102 can be substituted with, for example, isoleucine, such that the modified EPSPS gene includes a T102I substitution. The proline at amino acid 106 can be substituted with, for example, alanine, serine, threonine, glycine, cysteine, isoleucine, valine, methionine, or leucine. For example, the modified EPSPS gene can include a P106A substitution, a P106C substitution, a P106S substitution, or a P106I substitution. The glycine at amino acid 144 can be substituted with, for example, asparagine or aspartic acid, such that the modified EPSPS gene includes a G144D substitution or a G144N substitution. The alanine at amino acid 192 can be substituted with, for example, threonine, such that the modified EPSPS gene includes an A192T substitution. In some embodiments, a glyphosate tolerant plant having an altered expression profile of an EPSPS gene as described herein can include a modified EPSPS gene having two or more modifications in the coding sequence of the EPSPS gene. For example, a modified EPSPS gene can include a G101A substitution and an A192T substitution, a T102I substitution and a P106A substitution, a T102I substitution and a P106C substitution, a T102I substitution and a P106I substitution, a T102I substitution and a P106S substitution, or a G101A substitution and a G144D substitution. EPSPS amino acid numbering is relative to mature plant EPSPS proteins and as used elsewhere (see, e.g., Sammons and Gaines, 2014 Pest Management Science 70: 1367-1377).

Exemplary modified EPSPS enzymes include, for example, a corn EPSPS enzyme including one or more of the modifications set forth in SEQ ID NO:22, a cassava EPSPS enzyme including one or more of the modifications set forth in SEQ ID NO:24, a rice EPSPS enzyme including one or more of the modifications set forth in SEQ ID NO:26, an Arabidopsis EPSPS enzyme including one or more of the modifications set forth in SEQ ID NO:28, and a petunia EPSPS enzyme including one or more of the modifications set forth in SEQ ID NO:30.

A glyphosate tolerant plant having an altered expression profile of an EPSPS gene (e.g., a modified EPSPS gene) as described herein can also include a modification providing an alternative mechanism of glyphosate tolerance as described elsewhere (see, e.g., Sammons et al., 2014 Pest Manag. Sci. 70:1367-1377).

A glyphosate tolerant plant having an altered expression profile of an EPSPS gene (e.g., a modified EPSPS gene) as described herein can also include a modification useful for transporting the expressed EPSPS enzyme. EPSPS needs to be translocated to the chloroplasts, found in stems and leaves, to function. The addition of a sequence to the coding region which places a chloroplast transit peptide (CTP) on the EPSPS enzyme can direct the EPSPS protein to the chloroplasts in the plant cell.

A glyphosate tolerant plant having an altered expression profile of an EPSPS gene (e.g., a modified EPSPS gene) as described herein can also include a modification that confers additional (e.g., stacked) herbicide tolerance. Continuous use of single mode of action herbicide chemistries can lead to the development of herbicide resistant weeds and/or minimization of the value in those areas for a crop resistant to those same herbicides. Additional herbicide tolerance can include tolerance against any appropriate herbicide (e.g., agricultural herbicides) including, without limitation, acetolactate synthase (ALS) inhibitors, glufosinate, 2,4-D, and dicamba. For example, a glyphosate tolerant plant having an altered expression profile of an EPSPS gene (e.g., a modified EPSPS gene) as described herein can also be resistant to one or more ALS inhibitors. Resistance to one or more ALS inhibitors can be conferred by any appropriate method.

Any appropriate method can be used to introduce one or more modifications into an EPSPS regulatory sequence and/or into an EPSPS coding sequence to produce a glyphosate tolerant plant having an altered expression profile of an EPSPS gene (e.g., a modified EPSPS gene) as described herein. For example, genome editing can be used to produce a glyphosate tolerant plant (e.g., a nontransgenic glyphosate tolerant plant). Genome editing, or genome editing with engineered nucleases (GEEN) inserts, replaces, or removes DNA from a genome using one or more site-specific nucleases (SSN) and, in some cases, a repair template (RT). Nucleases can be targeted to a specific position in the genome, where their action can introduce a particular modification to the endogenous sequences. For example, a SSN can introduce a targeted double-strand break (DSB) in the genome, such that cellular DSB repair mechanisms incorporate a RT into the genome in a configuration that produces heritable glyphosate resistance in the cell, in a plant regenerated from the cell, and in any progeny of the regenerated plant. Nucleases useful for genome editing include, for example, zinc finger nucleases (ZFNs), transcription activator-like effector nucleases (TALE nucleases), CRISPR CRISPR-associated nucleases such as Cas9 or Cpf1, and homing endonucleases (HE; also referred to as meganucleases).

A RT can include right and left homology arms to mediate the process of DSB repair by homologous recombination (HR). The DSB repair process can include repair by HR or by insertion of the RT through non-homologous end joining (NHEJ), or by some combination of the two pathways, or by an unknown pathway. A RT can be designed to include one or more modifications to an EPSPS regulatory sequence and/or coding sequence of an EPSPS gene as described herein. A RT can include an EPSPS gene or a fragment of an EPSPS gene. In embodiments where a RT includes a fragment of an EPSPS gene, incorporation of the RT into the genome results in an edited genome that can encode an EPSPS polypeptide. In some embodiments, for example, a RT can be designed to include one or more modifications to an EPSPS regulatory sequence as described herein, as well as one or more modifications in the coding sequence of an EPSPS gene, or a fragment thereof, as described herein.

Other appropriate methods can be used to introduce one or more modifications into an EPSPS regulatory sequence and/or into an EPSPS coding sequence, to produce a glyphosate tolerant plant having an altered expression profile of an EPSPS gene (e.g., a modified EPSPS gene), as described herein. For example, one or more modifications can be made to an EPSPS gene regulatory sequence and/or to an EPSPS coding sequence without an SSN, by providing a RT that will modify the appropriate chromosomal target without the introduction of a DSB. Alternatively, one or more modifications can be made to an EPSPS gene regulatory sequence and/or to an EPSPS coding sequence without a RT, by introducing one or more SSNs that cut the plant genome in such a way as to cause a rearrangement of the endogenous sequence. Such modification can result in EPSPS gene expression being driven by a different promoter than that which was driving its expression before the modification.

The gene editing events described herein can be performed in appropriate plant cells or within any appropriate portion of a plant cell. For example, gene editing can be performed in the nucleus. In some cases, gene editing to introduce a modified EPSPS gene under the control of any appropriate promoter as described herein can be performed in a plastid of a plant.

The gene editing reagents described herein can be introduced into a plant by any appropriate method. In some cases, nucleic acids encoding the gene editing reagents can be introduced into a plant cell using Agrobacterium or Ensifer mediated transformation, particle bombardment, liposome delivery, nanoparticle delivery, electroporation, polyethylene glycol (PEG) transformation, or any other method suitable for introducing a nucleic acid into a plant cell. In some cases, the SSN or other expressed gene editing reagents can be delivered as RNAs or as proteins to a plant cell and the RT, if one is used, can be delivered as DNA.

A glyphosate tolerant plant having an altered expression profile of an EPSPS gene (e.g., a modified EPSPS gene) as described herein can be identified by, for example, glyphosate selection, selection for another introduced marker (e.g., NPTII, bar, or hpt), or by molecular screening (such as by PCR) of regenerated tissues.

This document also provides methods of optimizing glyphosate tolerance in a glyphosate tolerant plant described herein. For example, a method for optimizing the resistance to glyphosate in crop plants as an outcome of editing the plant's native EPSPS gene.

The invention will be further described in the following examples, which do not limit the scope of the invention described in the claims.

### Examples

### Example 1: Tissue-Specific Expression of the EPSPS Promoter

To examine the strength of expression caused by the native EPSPS promoter in various parts of the plant, a variety of tissues were dissected from TME204 cassava (Manihot esculenta) and tested for the abundance of the EPSPS mRNA by real time reverse-transcriptase quantitative PCR (qPCR).

Tissues were dissected from healthy cassava plants and total RNA was extracted for analysis. For leaf tissue and friable embryogenic callus (FEC), total RNA was isolated using the Spectrum Plant Total RNA kit (Sigma) and manufacturer's protocol. For shoot apical meristem, total RNA was isolated using the Ovation Pico WTA System V2 and manufacturer's protocol. For root samples, RNA was isolated using CTAB buffer extraction. Contaminating genomic DNA was removed from all samples with the TURBO DNA-free kit (Life Technologies) and manufacturer's protocol. cDNA synthesis was performed with SuperScript III reverse transcriptase (Life Technologies) and manufacturer's protocol. Quantitative PCR was performed with the SYBR Green PCR Master Mix (Life Technologies) and manufacturer's protocol to determine EPSPS expression in each tissue.

Following cDNA synthesis, quantitative PCR was performed to determine the relative expression of EPSPS. Expression of EPSPS was normalized to that of the reference gene GTPb. The following primers were used to amplify the EPSPS mRNA: forward 5′-GCTTCGAGTGCCAGTTATTTCCTGG-3′ (SEQ ID NO:50), reverse 5′-CAAGCACTTCAGCAAACCTTACATC-3′ (SEQ ID NO:51). The following primers were used to amplify GTPb mRNA: forward 5′-GTTGCCTTCTTTTGCGTTTCT-3′ (SEQ ID NO:52), reverse 5′-GCAATTTGATCCGTTTTCCAT-3′ (SEQ ID NO:53).

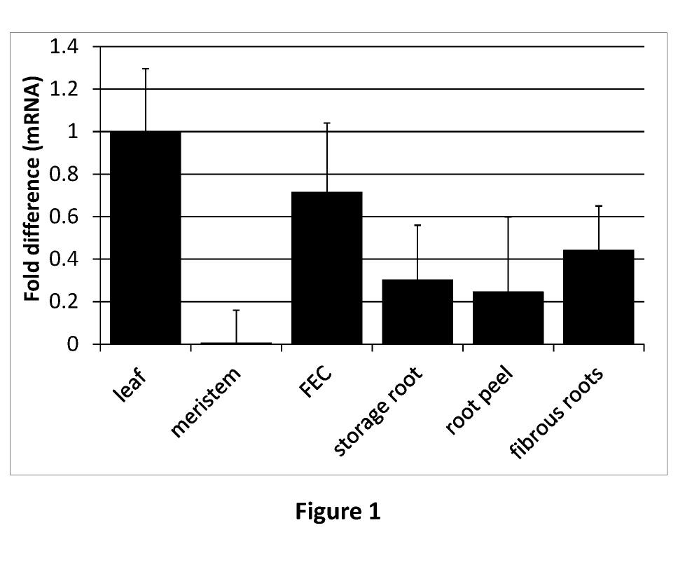

EPSPS expression was highest in leaves and lowest in the meristems (FIG. 1). Glyphosate accumulates in rapidly growing and developing tissues, such as meristems.

These results demonstrated that expression from the native EPSPS promoter may not be a sufficient to attain robust herbicide resistance in edited plants.

### Example 2: EPSPS Gene Models

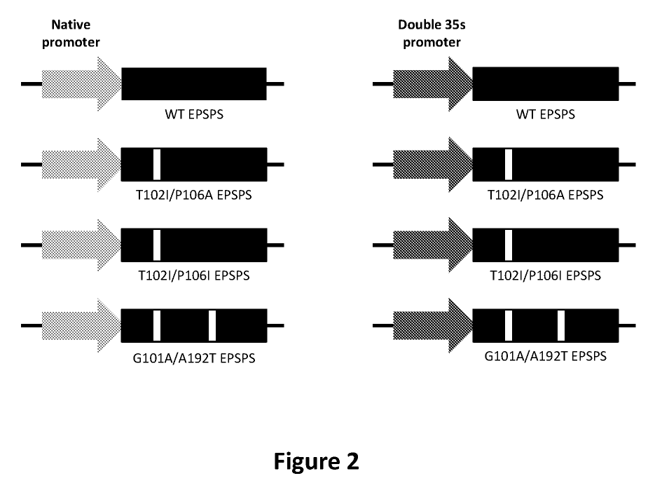

To investigate whether it is beneficial to express plant EPSPS genes more strongly than what is produced from the native promoter, a series of gene models (FIG. 2) were generated to compare the WT enzyme with three different amino acid substitution variants and each gene was expressed either from the TME7 cassava EPSPS promoter or from a double version of the Cauliflower Mosaic Virus 35S promoter for strong constitutive expression.

Gene models were assembled by PCR amplification and Gibson assembly in a pCAMBIA T-DNA vector backbone with a C-terminal 3× FLAG epitope tag for Agrobacterium transformation and plant regeneration to facilitate testing for glyphosate resistance at the tissue and whole-plant levels. The T-DNA contained the NPTII plant selectable marker driven by the nopaline synthase promoter, and events were selected during plant regeneration on media containing paromomycin. See SEQ ID NOs: 1-8 for the vector sequences. The transgenic plants were recovered following the method described by Chauhan et al., Plant Cell Tissue and Organ Culture 121: 591-603, 2015. A total of 17, 48, 24, 29, 19, 34, 16 and 16 rooted independent transgenic events were recovered form tissues transformed with constructs H001, H003, H002, H004, H009, H010, H013 and H014, respectively. H001, WT EPSPS with native promoter; H002, T102I/P106AEPSPS with native promoter; H003, WT EPSPS with 2×35s promoter; H004, T102I/P106AEPSPS with 2×35s promoter; H009, G101A/A192T EPSPS with native promoter; H010, G101A/A192T EPSPS with 2×35s promoter; H013, T102I/P106I EPSPS with native promoter; H014, T102I/P106I EPSPS with 2×35s promoter.

### Example 3: Expression of EPSPS Gene Models in Regenerated Plants

To ensure plants derived from the gene model transformation events would be suitable for glyphosate resistance testing, the expression of the EPSPS genes was assessed by semi-quantitative reverse-transcriptase PCR (sqPCR).

The following constructs were used: H001, WT EPSPS with native promoter; H002, T102I/P106A EPSPS with native promoter; H003, WT EPSPS with 2×35s promoter; H004, T102I/P106A EPSPS with 2×35s promoter; H009, G101A/A192T EPSPS with native promoter; H010, G101A/A192T EPSPS with 2×35s promoter; H013, T102I/P106I EPSPS with native promoter; H014, T102I/P106I EPSPS with 2×35s promoter.

Total RNA was isolated from leaf tissue for five transgenic lines derived from independent events with each construct with the Spectrum Plant Total RNA kit using the manufacturer's protocol. Genomic DNA contamination was removed from the RNA samples with the TURBO DNA-free kit and manufacturer's protocol. cDNA synthesis was performed with SuperScript III reverse transcriptase and manufacturer's protocol.

Following cDNA synthesis, expression of the EPSPS gene models was determined by semi-quantitative PCR using a forward primer (5′-TTGCAATTTGCACAGAGCTCAGG-3′; SEQ ID NO:54) that anneals in exon 7 of EPSPS and a reverse primer (5′-TCGTGGTCCTTGTAGTCGCC-3′; SEQ ID NO:55) that anneals in the 3× FLAG epitope tag that was included as a C-terminal fusion with the EPSPS gene models. These primers do not amplify the native EPSPS gene making them suitable to assess expression of the EPSPS gene models. Expression was compared to that of the reference gene GTPb. To verify that genomic DNA contamination was not interfering with the results, each sample was also tested in a negative control reaction (GTPb (-RT)) lacking reverse transcriptase.

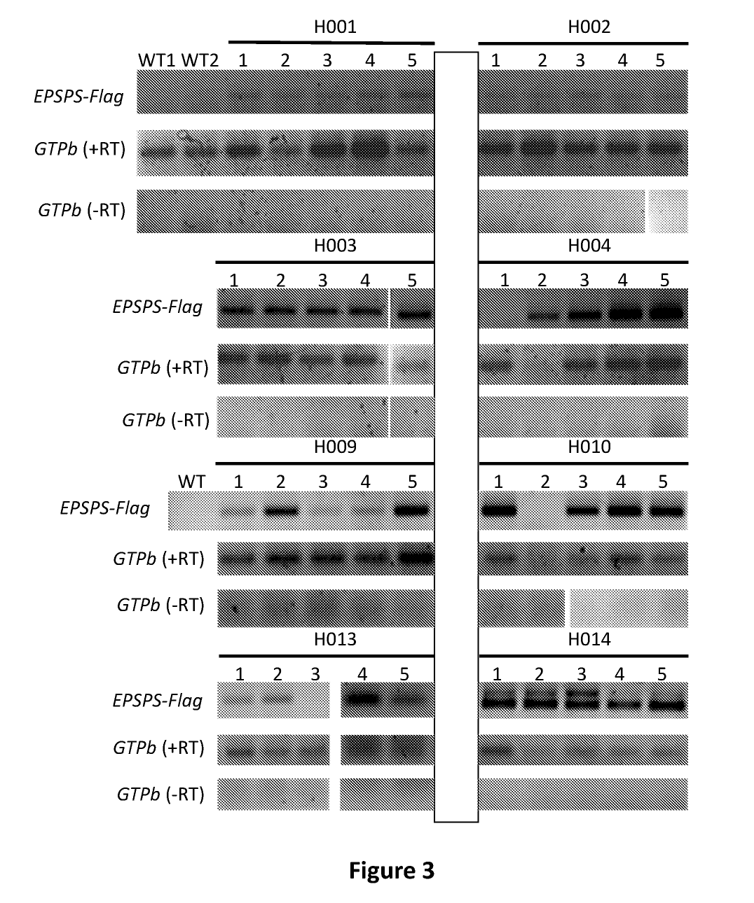

The majority of events derived from gene models with the double 35s promoter showed substantially stronger expression of the EPSPS mRNA than those derived from gene models with the cassava EPSPS promoter (FIG. 3).

These results demonstrate that EPSPS gene models can be expressed at high levels from the double 35s promoter.

### Example 4: Expression of EPSPS and Glyphosate Sensitivity

Experiments were conducted to access the effects of glyphosate on in vitro rooting by culturing non transgenic shoots on media containing different concentrations of glyphosate ranging from 0.001 mM to 1.0 mM.

Gene models are shown in FIG. 2, and indicated as follows: H001, WT EPSPS driven by the TME7 EPSPS promoter; H002, T102I/P106A EPSPS driven by the TME7 EPSPS promoter; H003, WT EPSPS driven by the 2×35s promoter; H004, T102I/P106A EPSPS driven by the 2×35s promoter.

Plantlets were regenerated from five independent transformation events with each gene model vector. Apical stem cuttings approximately 1.5 cm in length were excised from in vitro shoot cultures of transgenic plant lines and cultured on Murashige and Skoog basal media supplemented with 0.05 mM glyphosate. A total of five Petri dishes were established per transgenic event with three plantlets cultured in each dish.

Plants were monitored for a period of three weeks. The number of roots formed, length of roots, shoot vigor, and overall plant health were recorded at weekly intervals.

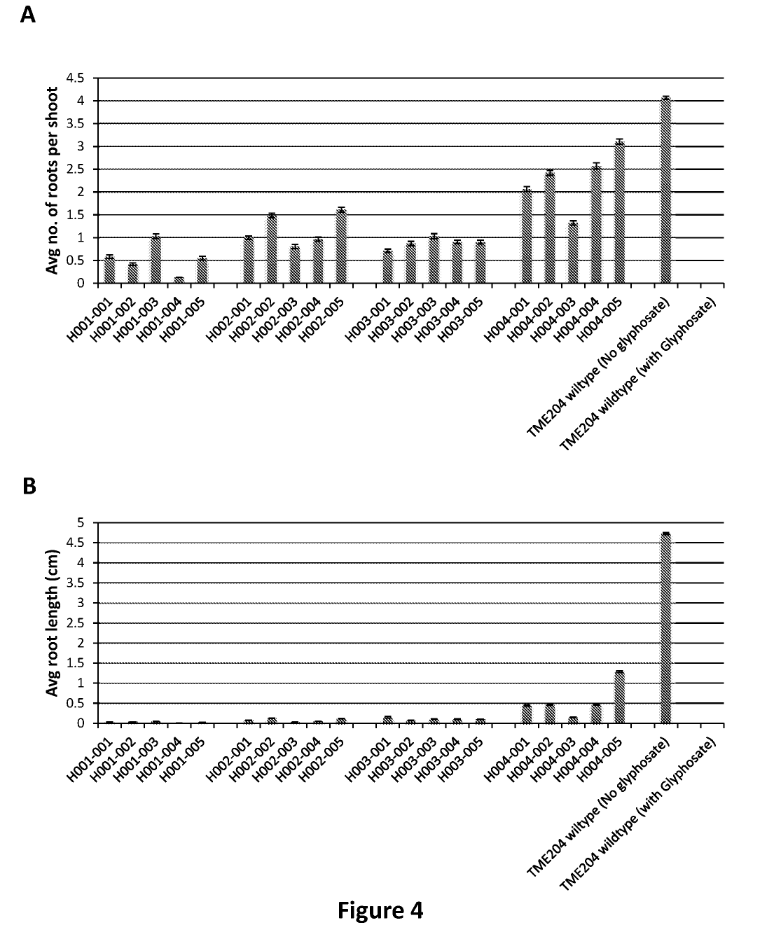

Inhibited root formation and shoot death was observed in non-transgenic stem cuttings when glyphosate was added to the medium at 0.05 mM. This assay was performed twice, and results of both experiments were combined, resulting in the data shown in FIG. 4. Therefore, selected independent transgenic events were cultured on media containing 0.05 mM glyphosate. The testing demonstrated that significantly (p=0.00) more and longer roots were formed from shoots of transgenic events expressing high levels of the T102I/P106A (TIPA) enzyme on glyphosate-containing media as compared to those expressing the gene variant at lower levels.

These results demonstrate that plants with highly expressed EPSPS gene variants are less sensitive to glyphosate during in vitro rooting.

### Example 5: Expression of EPSPS and Recovery of Transgenic Events with Glyphosate Selection

FEC was transformed with the EPSPS gene models to produce plants in which the EPSPS sequence was driven by the 2×35S promoter or the native EPSPS cassava promoter.

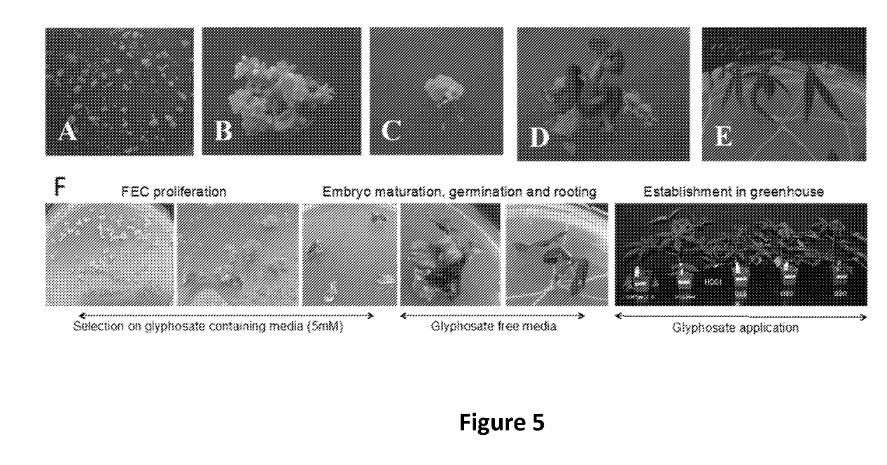

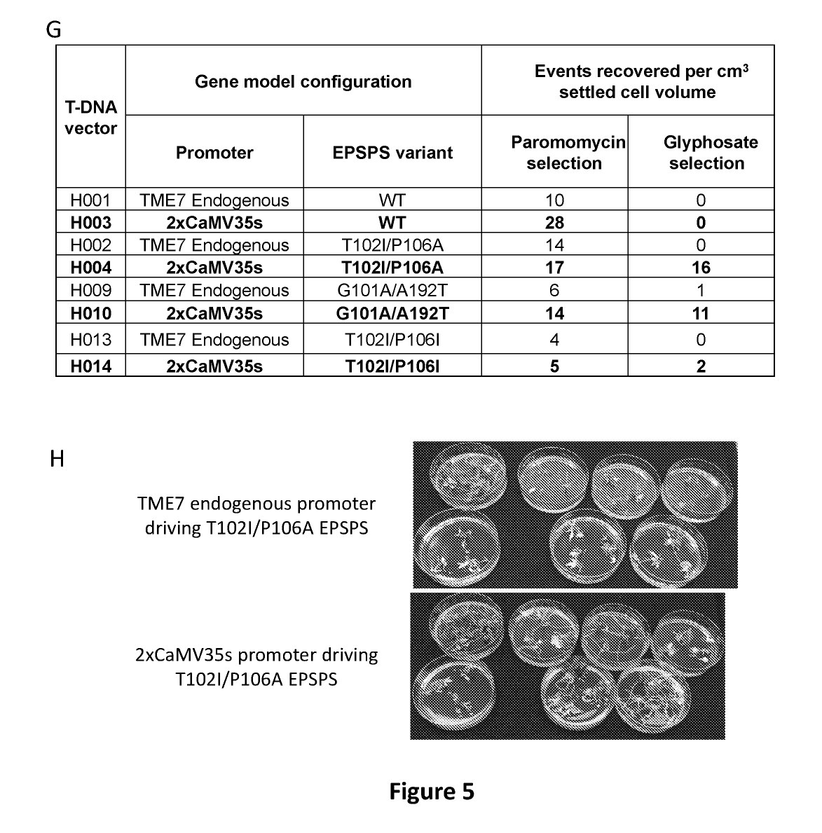

FEC transformed with EPSPS gene models were selected on media containing glyphosate in the range of 2.5—5.0 mM. Callus lines were recovered on media containing 2.5 mM glyphosate and transferred to embryo regeneration medium containing 5 mM glyphosate. Regenerated cotyledon-stage embryos were subcultured onto glyphosate free medium for germination and plantlet establishment (FIGS. 5A-F).

Selection of de novo gene model transformation events on media supplemented with glyphosate yielded 29 independent events as rooted plants from tissues expressing 2×35S driven EPSPS variants compared to only one event derived from a gene model expressing the EPSPS variant from the TME7 EPSPS EPSPS promoter (FIG. 5G). The TIPA, TIPI, and GAAT EPSPS variants driven by the 2×35s promoter all produced similar numbers of events as with selection by paromomycin (selection for the NPTII cassette, which was also present in the T-DNA of all gene models), indicating that these gene configurations are suitable for use as selectable markers during plant transformation.

These results demonstrated that highly expressed EPSPS gene variants produce more de novo independent transgenic events recovered under glyphosate selection than EPSPS gene variants expressed from the EPSPS promoter, indicating that glyphosate tolerance is increased with higher expression of the EPSPS enzymes.

### Example 6: Expression of EPSPS and Greenhouse Applications of Glyphosate

Plants transgenic for 2×35S and native EPSPS promoter expression cassettes were established in the greenhouse following procedures described elsewhere (Taylor et al., 2012 Tropical Plant Biology, 5: 127-139; and Chauhan et al., 2015 Plant Cell Tissue and Organ Culture 121: 591-603). Glyphosate 5.4 was prepared in a solution containing Surface surfactant and applied to leaves of 4-5 week old plants. Glyphosate was applied at a rate of 50 mg active ingredient per plant in a dropwise manner to the youngest 1-2 fully expanded leaves. Plants were grown on the greenhouse bench under conditions described elsewhere (Taylor et al., 2012 Tropical Plant Biology, 5: 127-139; and Chauhan et al., 2015 Plant Cell Tissue and Organ Culture 121: 591-603). Three times per week plants were scored visually on a 1-7 scale for damaged caused by the glyphosate application, where 1=no damage and 7=death.

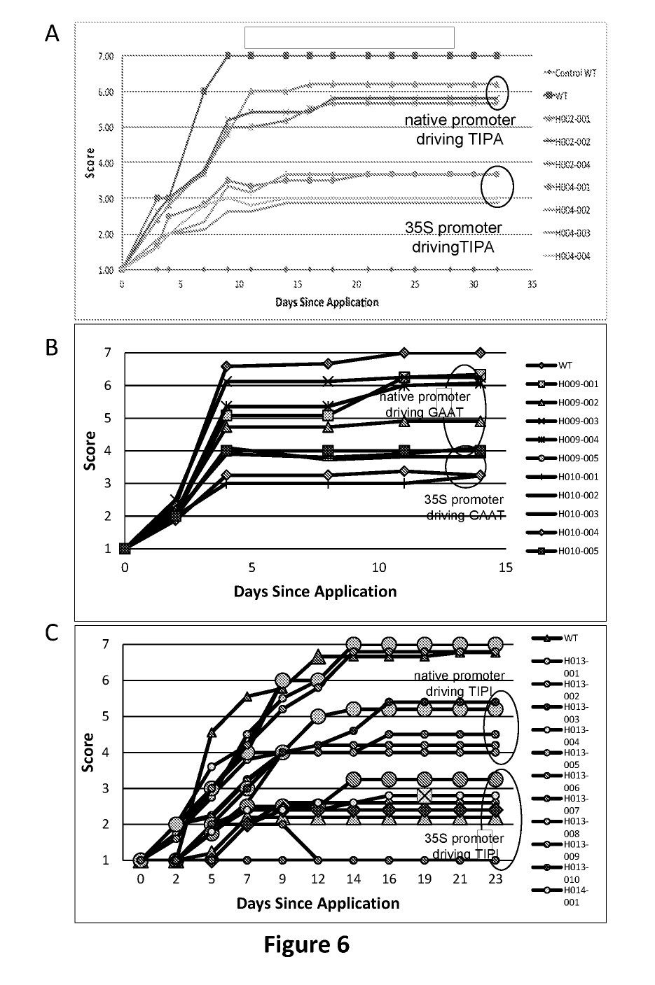

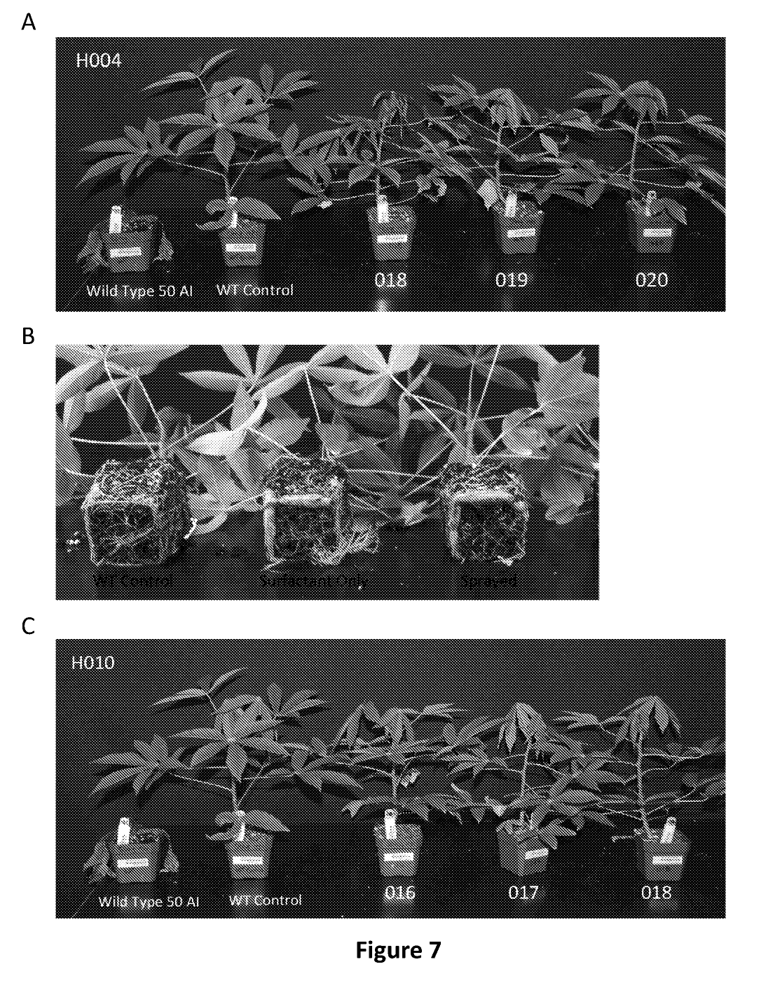

Regardless of the EPSPS amino acid substitution variants tested, plants in which the 35S promoter drove EPSPS expression were significantly more tolerant to herbicide treatment than plants in which the EPSPS promoter drove expression of the enzyme. Plants transgenic for H004 (double 35s promoter driving the TIPA enzyme) averaged a damage score of less than 3 while plants expressing the same enzyme under control of the native EPSPS promoter (H002) averaged a damage scale of approximately 6 in the same assay (FIG. 6A). Similarly, plants transgenic for H010 (double 35s promoter driving the GAAT enzyme) averaged a damage score of 3-4, while plants expressing the same enzyme under control of the native EPSPS promoter (H009) averaged a damage scale between 4 and 6 in the same assay (FIG. 6B). Finally, plants transgenic for H014 (double 35s promoter driving the TIPI enzyme) averaged a damage score of 1 to approximately 3, while plants expressing the same enzyme under control of the native EPSPS promoter (H013) averaged a damage scale between 4 to approximately 5.5 in the same assay (FIG. 6C). A small number of lines transformed with the H009, H010, H013, and H014 gene models showed no resistance to glyphosate. However, these lines were not extensively characterized prior to herbicide challenge, so they likely do not contain a functional copy of the EPSPS cassettes, or exhibit low expression of the enzyme. FIG. 7 shows representative phenotypes of H004 and H010 events after herbicide challenge, compared to complete killing the susceptible control.

These results demonstrated that regardless of amino acid substitution combinations used, plants with highly expressed EPSPS gene variants show superior glyphosate tolerance compared to the same EPSPS variants expressed from the EPSPS promoter.

### Example 7: Identification of Strong Constitutive Promoters for Driving EPSPS Expression

RNA sequencing was performed to assess global gene expression in cassava and identify promoters with strong constitutive expression.

RNA from 11 different tissues was examined: leaf blade, leaf midvein, petiole, stem, lateral bud, shoot apical meristem (SAM), root apical meristem (RAM), fibrous roots, storage root, friable embryogenic callus (FEC), and organized embryogenic structures (OES). Three separate biological replicates were isolated for each tissue. For tissues other than apical meristems, total RNA was isolated from leaf tissue with the Spectrum Plant Total RNA kit and manufacturer's protocol. For SAM and RAM, total RNA was isolated with the PicoPure RNA Isolation kit (Arcturus) and manufacturer's protocol. Genomic DNA was removed from the RNA samples with the TURBO DNA-free kit and manufacturer's protocol. RNA quality was assessed on an Agilent Bioanalyzer.

For library preparation with tissues other than SAM and RAM, 5 μg of RNA was used as input. For SAM and RAM tissues, six samples were pooled to get a total of 500-600 ng of RNA from each. The NEBNext Poly(A) mRNA Magnetic Isolation Module (New England BioLabs) was used to isolate mRNA, which was then used for library prep using NEBNext mRNA Library Prep Master Mix Set for Illumina (New England BioLabs) with 13 cycles of PCR amplification. Standard library prep protocol was followed for all samples except for the SAM and RAM, in which 1 μL of fragmentation enzyme was used instead of 2 μL and 0.5 μL of random primer was used instead of 1 μL. Library quality was assessed with the Agilent Bioanalyzer and quantified by Qubit. In total, 33 RNAseq libraries were made from 11 different tissue types with 3 biological replicates each. All libraries were multiplexed into one lane of Illumina HiSeq 2500.

The sequencing reads from each sample were cleaned with Trimmomatic version 0.32. Using tophat2 version 2.1.0, these cleaned reads were then mapped to the version 6.1 draft assembly of Manihot esculenta AM560-2 provided on Phytozome10.3 (http://phytozome.jgi.doe.gov/pz/portal.html). The read mapping output was then linked into candidate gene models for each sample using Cufflinks version 2.2.1. The gene models from all samples of the experiment were merged into one gene model file using Cuffmerge version 2.2.1. Using the output from Cuffmerge and the read mapping files from each sample, a differential expression analysis between tissue types was performed using Cuffdiff version 2.2.1. The output of Cuffdiff was processed in Python with the pandas package to visualize the expression data. EPSPS expression in leaf tissue is 110 fragments per kilobase of transcript per million mapped reads (FPKM).

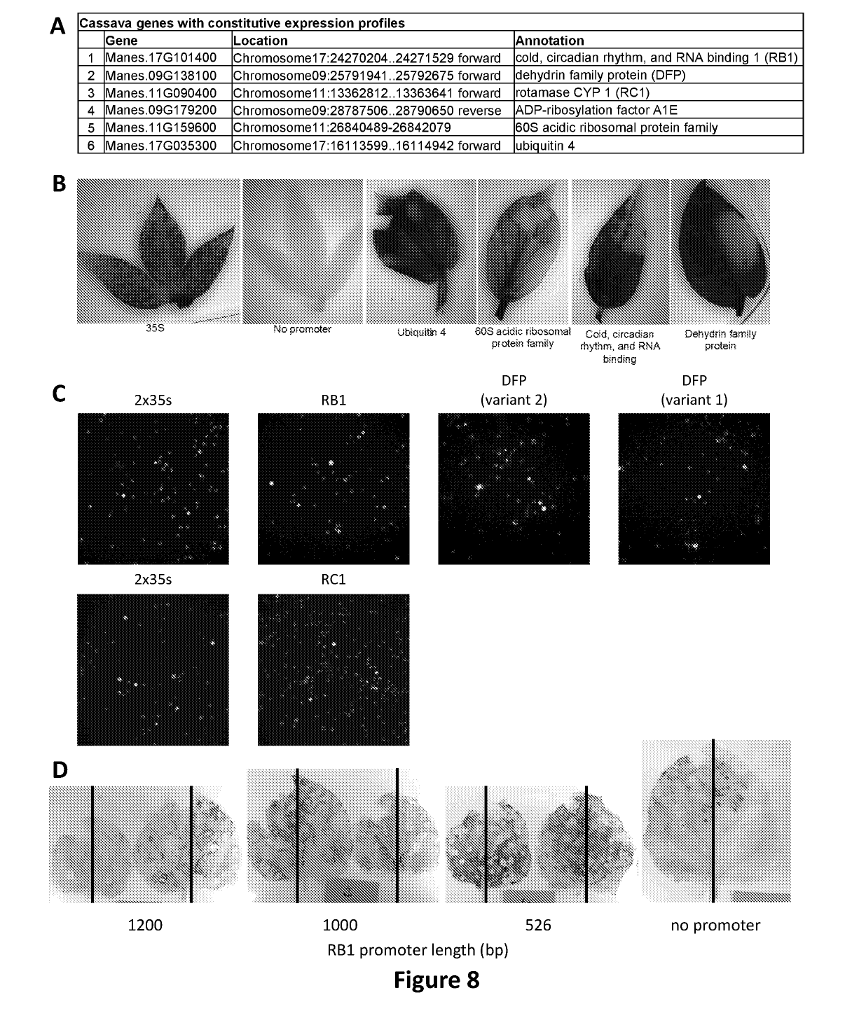

Six annotated genes (FIG. 8A) expressed in every tissue and having expression value of at least 200 FPKM (approximately two-fold greater than that of EPSPS in leaf tissue) for all 11 tissues studied were identified as having candidate promoters that may be useful for driving EPSPS expression. Several of these promoters were cloned and tested for their ability to drive expression of the β-Glucuronidase (GUS) gene in N. benthamiana leaves, in cassava FEC (Figures S2 and 5, respectively, of Wilson et al., 2016 bioRxiv preprint doi: http://dx.doi.org/10.1101/073213), in cassava leaves (FIG. 8B), and in cassava protoplasts (FIG. 8C).

To test for strong expression outside of their native genomic context, several promoters were PCR-amplified and cloned into a plasmid-borne eGFP expression cassette followed by transfection into cassava leaf protoplasts. Cells were prepared from leaf tissue of approximately 24 in vitro plants (Taylor et al., 2012 Tropical Plant Biology, 5: 127-139, and Chauhan et al., 2015 Plant Cell Tissue and Organ Culture 121: 591-603). Cells were isolated and transfected as described for tobacco protoplasts (Zhang et al., 2013 Plant Physiology, 161: 20-27) except for the sliced leaf tissue which was digested approximately 14 hours. Cells were then filtered through a 40 μM filter, and incubated 48 hours in washing (W5) buffer after transfection.

Each sample was transfected with approximately 15 μg of plasmid DNA containing the eGFP gene driven by each identified cassava promoter. As shown in FIG. 7B, three promoters (a promoter driving expression of a Manes.17G101400 gene, a promoter driving expression of a Manes.09G138100 gene, and a promoter driving expression of a Manes.11G090400 gene) produced eGFP intensity in protoplasts comparable to that generated by the double 35s promoter, indicating strong expression when those promoters are removed from their native genomic context.

In all tests, promoters with the functional annotations of cold, circadian rhythm, and RNA binding 1 (RB1), and dehydrin family protein (DFP) drove expression of the reporter gene to the highest levels, and were comparable to expression levels achieve by the double 35s promoter. The RB1 promoter was further characterized by evaluating the ability of shorter fragments to drive expression of the GUS reporter in N. benthamiana leaves. T-DNAs delivering different length fragments of the RB1 promoter were Agroin filtrated in the right half of the young leaves, while a similar construct using the double 35s promoter was infiltrated into the left side. Based on the GUS expression strength shown in FIG. 8D, RB1 derivatives as short as 526 nucleotides were capable of expressing the reporter to similar levels as the double 35s promoter. For this reason, the RB1 promoter was selected for use in repair templates to swap it in for the EPSPS promoter during editing experiments.

Putative functional annotations were determined by phytozome from sequence similarity to A. thaliana TAIR10 gene models (Prochnik et al., 2012 Tropical plant biology. 5:88-94) along with the genome assembly from phytozome. For the stable cassava transformation, each promoter was operably linked to the GUS gene open reading frame and transformed into cassava by random T-DNA integration as described in Example 2 for the gene model constructs.

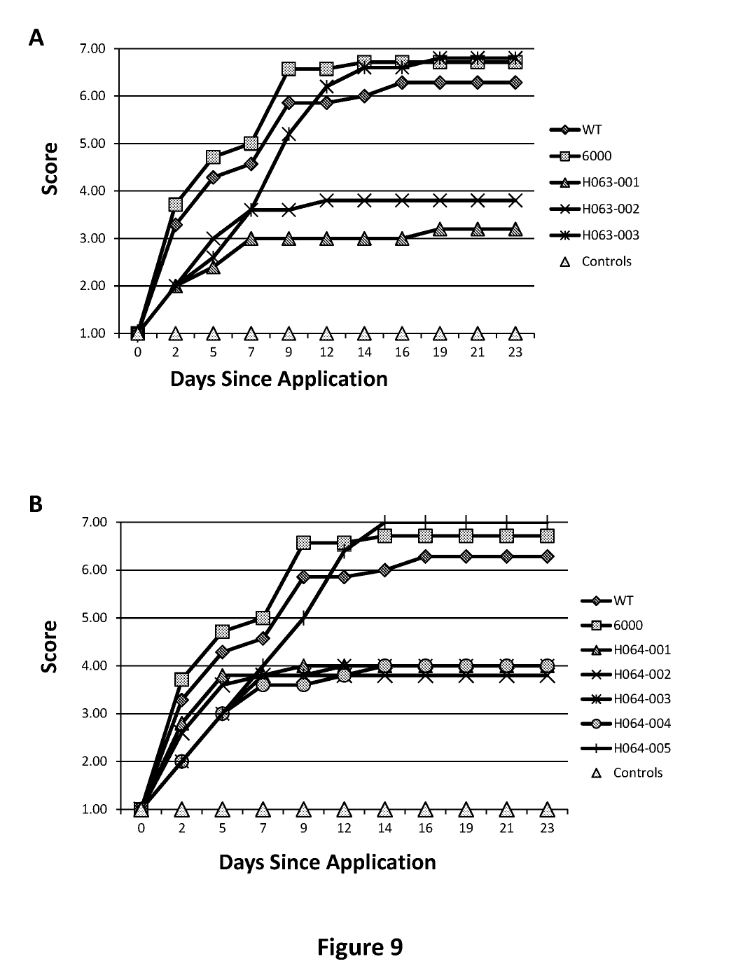

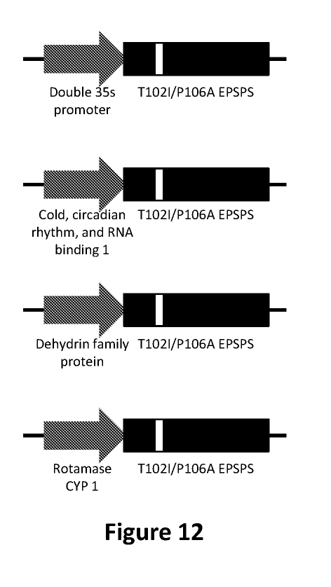

To test the suitability of the two best promoter candidates for driving EPSPS expression, the RB1 and DFP promoters were used to drive expression of TIPA gene models (as shown in FIG. 12) transformed into cassava as described for the other gene models in Example 2. Independent lines were challenged with glyphosate as described for the other gene models in Example 6. Both native promoters provided glyphosate tolerance comparable to that provided by the double 35s promoter driving the TIPA enzyme (compare the H063 and H064 performance in FIGS. 9A and 9B, respectively, to the H004 performance in FIG. 6A).

These results demonstrated that native plant promoters such the promoters shown in FIG. 7A (including promoters driving expression of genes with IDs Manes.17G101400, Manes.09G138100, and Manes.11G090400) can be used to drive strong constitutive expression of a glyphosate resistant, class I EPSPS enzyme without the incorporation of foreign DNA into a modified plant.

### Example 8: EPSPS Gene Editing in Protoplasts

Plasmids were constructed for delivery of gene editing reagents into plant cells on standard T-DNAs or on T-DNAs harboring a geminivirus derived replicon (see, e.g., WO 2013/192278) which amplifies the expression and copy number of the gene editing reagents after transfer into plant cells.

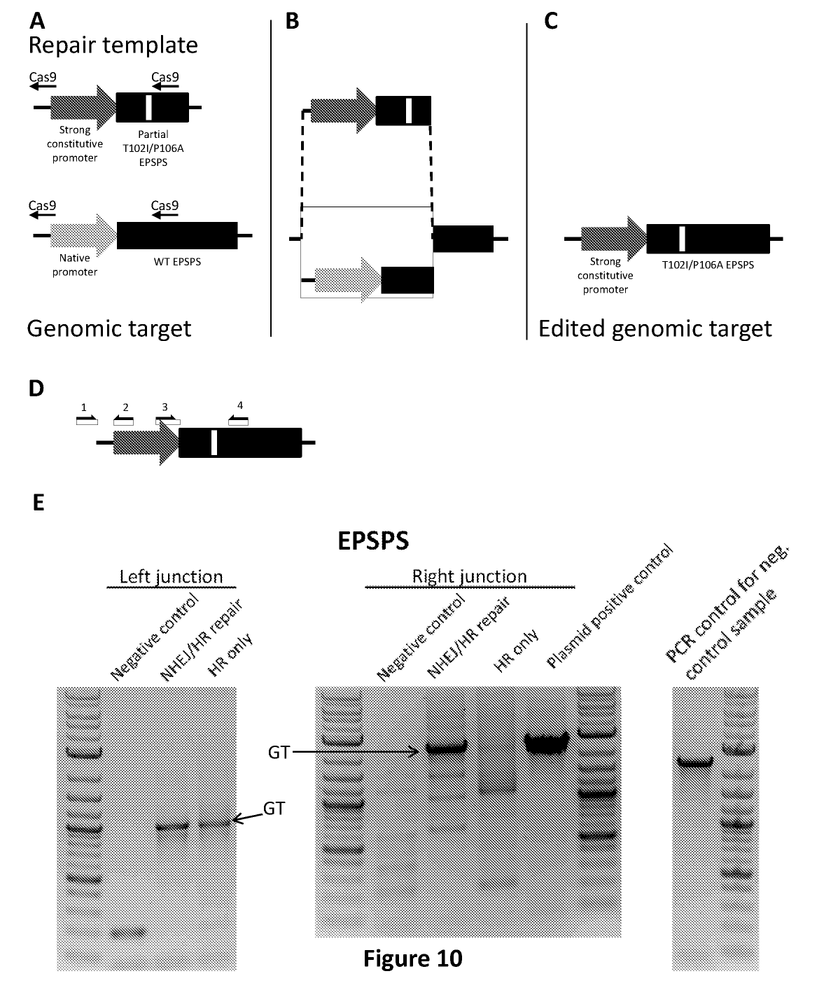

Each replicon carried Cas9 expressed by the double 35s promoter, and two RNA polymerase III promoters for the expression of two sgRNAs, one targeted approximately 1.7 kb upstream of the EPSPS start codon and the other targeted to the second intron approximately 150 bp downstream of exon 2. Cutting at both targets simultaneously resulted in excision of the EPSPS promoter and the first two exons of the gene. Each replicon also carried a repair template either without nuclease targets that was designed to repair the broken EPSPS allele by HR only (SEQ ID NO: 10), or a repair template with nuclease targets that was designed to repair the broken EPSPS allele by either HR or NHEJ (SEQ ID NO: 9). An example NHEJ repair event is illustrated in FIGS. 10A-C.

These vectors were transfected into fresh cassava protoplasts as described in Example 7. Two days after transfection, total genomic DNA was isolated and tested for gene editing by PCR using the strategy shown in FIG. 10D.

Primers were designed so that one primer specific for flanking genomic DNA was paired with another primer specific for sequence contained only in the repair template. Using this strategy, template suitable for PCR amplification was only present if the gene editing event had occurred in the correct orientation. Primer pairs specific for both the left and right junctions were used to test the isolated genomic DNA. As shown in FIG. 10E, expected bands were observed for both junctions in samples treated with gene editing plasmids, but not in samples treated with the control.

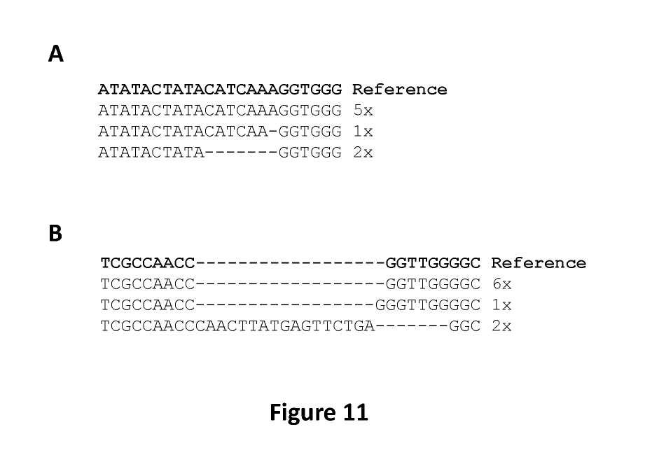

To verify that these bands represented useful gene editing events, individual clones derived from the amplicon pools from the vector using NHEJ or HR repair were sequenced at the left-junction (FIG. 11A) and the right-junction (FIG. 11B). In both cases the expected sequences are shown on top (“Reference”) which would result from perfect recombination of the cleaved RT into the genomic DSB without any loss or addition of nucleotides. All observed sequences are consistent with the expected NHEJ or HR repair scenarios for the intended gene editing events. See SEQ ID NO: 9 for the sequence of the plasmid used to cause the gene editing events shown in the protoplast population. Furthermore, all nine of the sequences shown in FIG. 11B represented right junction repair events that would result in proper expression of the T102I/P106A EPSPS (numbering relative to other mature plant EPSPS proteins consistent with Sammons and Gaines, 2014 Pest Management Science. 70: 1367-1377) edited gene from the double 35s promoter.

These results demonstrated successful gene editing in cassava protoplasts to modify the EPSPS gene by replacement of the native promoter with a strong constitutive promoter and to introduce amino acid substitutions that confer herbicide tolerance.

### Example 9: EPSPS Gene Editing and Regeneration of Herbicide Tolerant Plants

Editing of plant EPSPS genes for amino acid substitutions and strong constitutive expression can lead to robust resistance to glyphosate in recovered plants.

Vectors containing gene editing reagents described in Example 8 were introduced into cassava callus via Agrobacterium transformation (for example, SEQ ID NOs: 11-14). The vectors were designed to repair the EPSPS promoter excision event either by HR or NHEJ from a standard T-DNA (H055; SEQ ID NO: 11), by HR only from a standard T-DNA (H056; SEQ ID NO: 12), or by HR only from a geminivirus replicon (H060; SEQ ID NO: 14). Gene editing events using these reagents could be identified by selecting in culture for resistance to glyphosate. A second series of these same vector configurations was generated with an NPTII selectable marker cassette to enable paromomycin selection for genomic integration of the gene editing vector (H080 and H081). For these vectors, plants were first regenerated by selecting for paromomycin and then tested for gene editing at EPSPS.

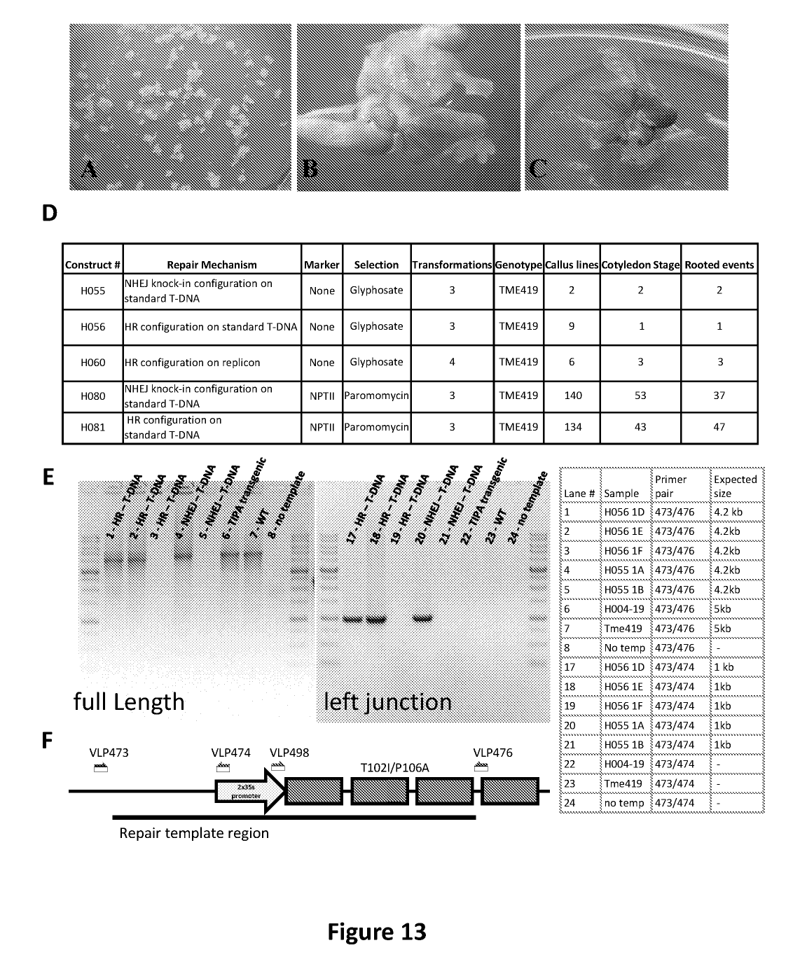

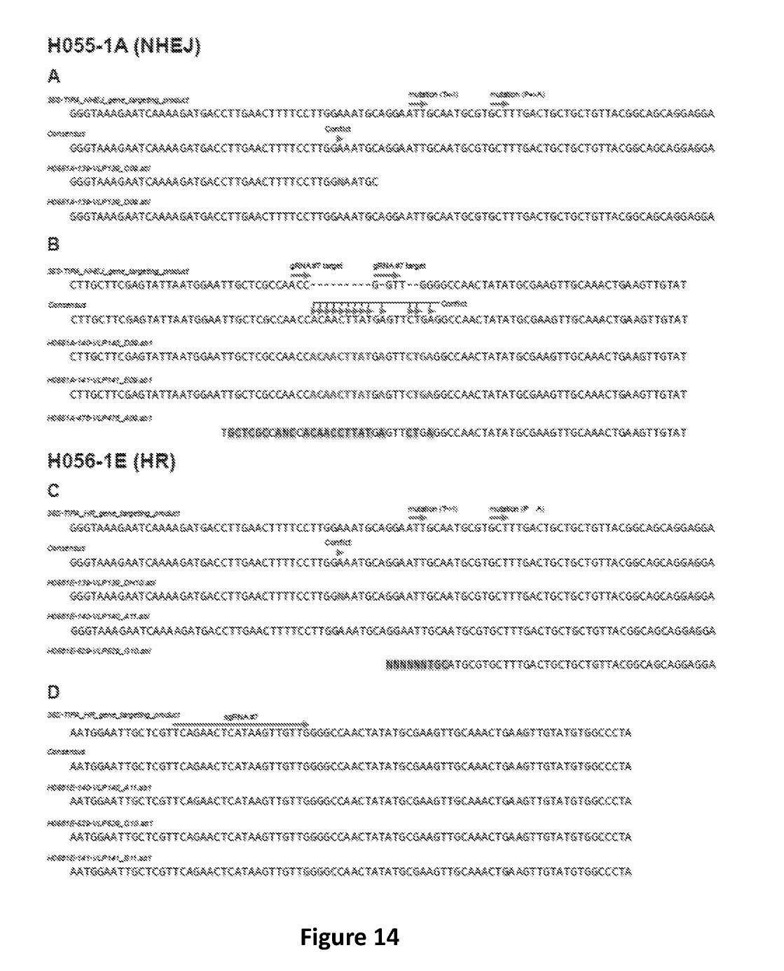

Gene editing events were recovered by plant regeneration in the presence of glyphosate or paromomycin. The methods described by Taylor et al., Tropical Plant Biology, 5: 127-139, 2012 and Chauhan et al., Plant Cell Tissue and Organ Culture 121: 591-603, 2015 were used for production of FEC, Agrobacterium mediated transformation and recovery of plants. Selection of EPSPS editing events (FIGS. 13A-C) was performed on media containing 2.5 and 5.0 mM glyphosate for the H055, H056 and H060 vectors and on paromomycin as described by Chauhan et al., 2015 for the H080 and H081 vectors. As shown in the table in FIG. 13D, glyphosate resistant events were obtained from all of the described repair template configurations and selection strategies.

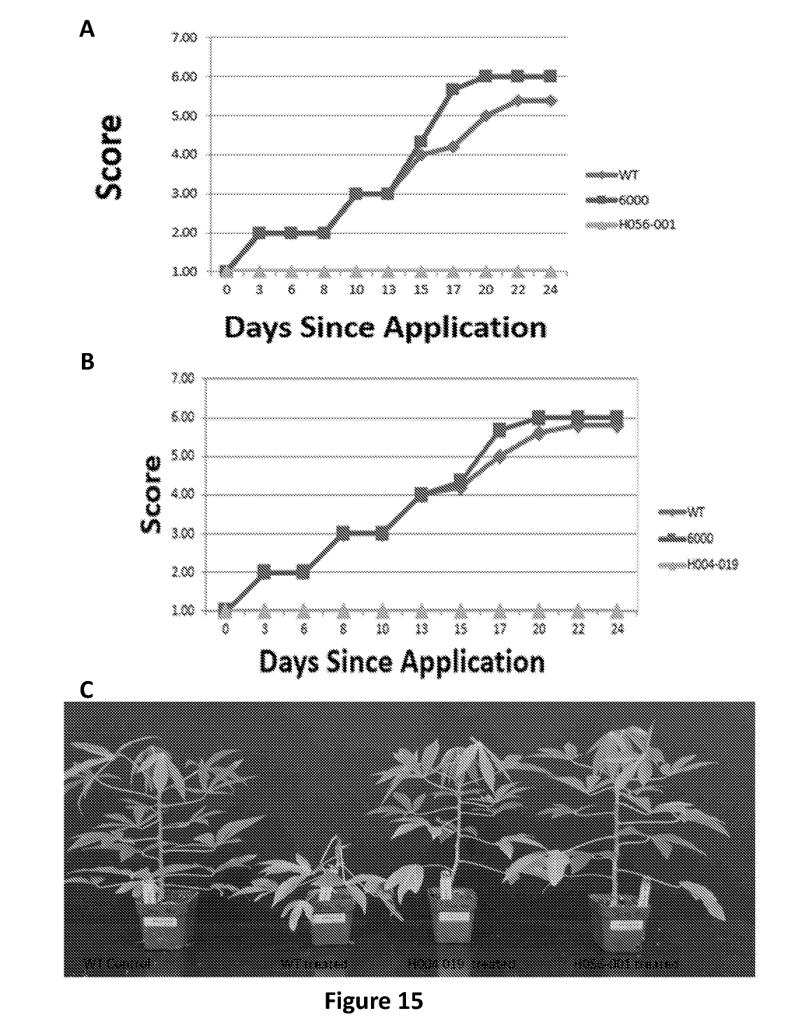

Putative EPSPS gene editing events were molecularly characterized by PCR for the presence of the left junction and the full length edited allele (FIGS. 13E-G) to verify presence of the expected editing events. Glyphosate resistance of one of the regenerated editing events was verified with the herbicide challenge assay described in Example 6. FIGS. 15A-C indicate the tolerance of the edited event is equivalent to the best H004 event recovered. Together these data indicate it is possible to edit a plant native EPSPS allele to produce strong constitutive expression of an EPSPS enzyme that provides robust herbicide tolerance in regenerated plants. The editing events can be made with or without geminivirus replicons, using either the HR or NHEJ double strand break repair pathways. The selective agent used to isolate and recover treated cells can be either glyphosate (to directly select for the successful editing event) or another suitable compound such as paromomycin (to select for cells with integrated gene editing reagents that can be molecularly screened for the editing event).

### Example 10: EPSPS Gene Editing and Regeneration of Herbicide Tolerant Plants with Native Strong Constitutive Promoters

To generate non-transgenic glyphosate resistant plants, it is possible to use promoters derived from the organism itself to drive EPSPS expression after a successful editing event. As an example of this, other vectors carrying the three strong cassava promoters validated in protoplasts in Example 7 are introduced into cassava callus. The gene editing reagents can be carried either on a standard T-DNA or on a T-DNA containing a geminivirus replicon and can employ either the NHEJ or HR pathways (for example, SEQ ID NOs: 15-20).

Possible EPSPS gene editing outcomes produced in cassava edited EPSPS alleles resulting from modification with the above described vectors are shown in FIG. 12

Native promoters can be incorporated into EPSPS alleles by gene editing and are useful for driving expression of plant EPSPS gene variants to produce glyphosate tolerance without incorporation of foreign DNA at the edited allele.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1. A method for generating a glyphosate tolerant plant, the method comprising:
site-specific editing of a plant genome in order to modify EPSPS gene regulation, wherein said site-specific editing comprises operably linking an EPSPS gene to an alternative promoter at a native genomic location of the alternative promoter, operably linking an EPSPS gene to a recombinant promoter at a native genomic location of the EPSPS gene, or introducing an enhancer into an endogenous EPSPS regulatory sequence,
wherein said modification to EPSPS gene regulation is effective to cause an altered EPSPS gene expression profile compared to an EPSPS gene expression profile of a non-edited plant genome, and
wherein said altered EPSPS expression profile confers glyphosate tolerance.

2. (canceled)

3. The method of claim 1, wherein said alternative promoter is selected from the group consisting of an actin promoter, a ubiquitin promoter, a histone promoter, a promoter that drives expression of Manes.17G101400 gene, a promoter that drives expression of Manes.09G138100, and a promoter that drives expression of Manes.11G090400.

4. The method of claim 1, wherein said alternative promoter is a constitutive promoter, and wherein said altered EPSPS gene expression profile comprises increased EPSPS expression.

5. (canceled)

6. The method of claim 1, wherein said alternative promoter is a meristem specific promoter selected from the group consisting of CLV3, FIL, and WUS, and wherein said altered EPSPS gene expression profile is increased EPSPS gene expression in a meristem.

7.-9. (canceled)

10. The method of claim 1, wherein said recombinant promoter is a constitutive promoter, and wherein said altered EPSPS gene expression profile comprises increased EPSPS expression.

11. (canceled)

12. The method of claim 1, wherein said alternative promoter is a meristem specific promoter selected from the group consisting of CLV3, FIL, and WUS, and wherein said altered EPSPS gene expression profile is increased EPSPS gene expression in a meristem.

13. (canceled)

14. The method of claim 1, wherein said recombinant promoter is an inducible promoter selected from the group consisting of Es and PR-1.

15. (canceled)

16. (canceled)

17. The method of claim 1, wherein the enhancer is selected from the group consisting of a 35s enhancer, a translational enhancer from the tobacco etch virus, and a pea PetE enhancer, and wherein said altered EPSPS gene expression profile is an increase in EPSPS expression.

18. The method of claim 1, wherein said EPSPS gene comprises a modified EPSPS coding sequence, wherein said modified EPSPS coding sequence comprises a substitution of the glycine at amino acid 101 (G101), a substitution of the threonine at amino acid 102 (T102), a substitution of the proline at amino acid 106 (P106), a substitution of the glycine at amino acid 144 (G144), a substitution of the alanine at amino acid 192 (A192), or any combination thereof.

19. (canceled)

20. The method of claim 18, wherein said modified EPSPS coding sequence comprises a T102I substitution and a P106A substitution, a G101A substitution and an A192T substitution, a T102I substitution and a P106C substitution, a T102I substitution and a P106I substitution, a G101A substitution and a G144D substitution, a G101A substitution and a G144N substitution, or a T102I substitution and a P106S substitution.

21.-38. (canceled)

39. A glyphosate tolerant plant comprising:
a genome edited in a site-specific manner to modify EPSPS gene regulation, wherein said site-specific editing comprises operably linking an EPSPS gene to an alternative promoter at a native genomic location of the alternative promoter, operably linking an EPSPS gene to a recombinant promoter at a native genomic location of the EPSPS gene, or introducing an enhancer into an endogenous EPSPS regulatory sequence;
wherein said modification to EPSPS gene regulation is effective to cause an altered expression profile of an EPSPS gene compared to an EPSPS gene expression profile of a non-edited plant genome;
wherein said altered expression profile of said EPSPS gene confers glyphosate tolerance.

40. The glyphosate tolerant plant of claim 39, wherein said modification to EPSPS gene regulation comprises introducing an enhancer into an EPSPS gene regulatory sequence, and wherein said altered EPSPS gene expression profile is an increase in EPSPS gene expression.

41. The glyphosate tolerant plant of claim 40, wherein the enhancer is selected from the group consisting of a 35s enhancer, a translational enhancer from the tobacco etch virus, and a pea PetE enhancer.

42. The glyphosate tolerant plant of claim 40, wherein the enhancer is introduced into an EPSPS promoter, into an EPSPS intron, upstream of an EPSPS promoter, or downstream of an EPSPS terminator.

43. The glyphosate tolerant plant of claim 39, wherein said edited genome comprises said EPSPS gene operably linked to an alternative promoter at a native genomic location of the alternative promoter, and wherein said altered EPSPS gene expression profile comprises increased EPSPS expression.

44. (canceled)

45. The glyphosate tolerant plant of claim 43, wherein said alternative promoter is selected from the group consisting of an actin promoter, a ubiquitin promoter, a histone promoter, a promoter that drives expression of Manes.17G101400 gene, a promoter that drives expression of Manes.09G138100, and a promoter that drives expression of Manes.11G090400.

46. (canceled)

47. The glyphosate tolerant plant of claim 39, wherein said alternative promoter is a meristem specific promoter selected from the group consisting of CLV3, FIL, and WUS, and wherein said altered EPSPS gene expression profile is EPSPS gene expression in a meristem.

48. (canceled)

49. (canceled)

50. The glyphosate tolerant plant of claim 39, wherein said recombinant promoter is a constitutive promoter selected from the group consisting of an actin promoter, a ubiquitin promoter, a histone promoter, a promoter that drives expression of Manes.17G101400 gene, a promoter that drives expression of Manes.09G138100, and a promoter that drives expression of Manes.11G090400, and wherein said altered EPSPS gene expression profile comprises increased EPSPS expression.

51. (canceled)

52. (canceled)

53. The glyphosate tolerant plant of claim 39, wherein said alternative promoter is a meristem-specific promoter selected from the group consisting of CLV3, FIL, and WUS, and wherein said altered EPSPS gene expression profile is increased EPSPS gene expression in a meristem.

54. (canceled)

55. The glyphosate tolerant plant of claim 39, wherein said recombinant promoter is an inducible promoter selected from the group consisting of Es and PR-1.

56. (canceled)

57. The glyphosate tolerant plant of claim 39, wherein said glyphosate tolerant plant is a monocotyledonous plant selected from the group consisting of maize, rice, wheat, barley, sugarcane, oat, rye, millet, sorghum, switchgrass, turfgrass, and bamboo.

58. (canceled)

59. The glyphosate tolerant plant of claim 39, wherein said glyphosate tolerant plant is a dicotyledonous plant selected from the group consisting of bean, soybean, cotton, pea, cowpea, peanut, almond, walnut, apple, plum, peach, pear, citrus, sugar beet, squash, melon, cassava, tomato, pepper, canola, banana, flax, and sunflower.

60. (canceled)

61. The glyphosate tolerant plant of claim 39, wherein said EPSPS gene comprises a modified EPSPS coding sequence, wherein said modified EPSPS coding sequence comprises a substitution of the glycine at amino acid 101 (G101), a substitution of the threonine at amino acid 102 (T102), a substitution of the proline at amino acid 106 (P106), a substitution of the glycine at amino acid 144 (G144), a substitution of the alanine at amino acid 192 (A192), or any combination thereof.

62. (canceled)

63. The glyphosate tolerant plant of claim 61, wherein said modified EPSPS coding sequence comprises a T102I substitution and a P106A substitution, a G101A substitution and an A192T substitution, a T102I substitution and a P106C substitution, a T102I substitution and a P106I substitution, a G101A substitution and a G144D substitution, a G101A substitution and a G144N substitution, or a T102I substitution and a P106S substitution.

64.-69. (canceled)


SEQUENCE LISTING (55 sequences)

SEQ ID NO: 1 - DNA, 17523 bp, artificial
  T-DNA vector with wildtype cassava TME7 promoter driving wildtype EPSPS
taatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaaaagctttcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtcgacagatctagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcgaattcgagctcggtacccggggatccgcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggaggcccgaaagtagtgaagcccagtctacgctatagtccgttttcttcctcgtcataatattcaatttgattaagaaaatggttctaataacgtcaatttttgaaaactttggtggattcaccaacttcattgatgtccaaaatgccaatttacatgacctgaaccgagcggtttgaataaacgggttgaatttgaacctagccatagcaggaagttcacaagataaggaatcaaatgatcgagacattggacgcatgattcggtttttaaaatcttactaaatcagcgttatcattaaagggtttaaatcacaactacacgtgctattcatttttttatattcagttatcatatctttatcatccatttaacatgaagtgtagattgctattaatattttatcaaactttaattattttattttattttaaatttaattaaaatatttttatatttataataacatataaatttatatgagtctttcaaaatcaaaacacttatacactattattgtaatctaaacaatcaccatagacttgagtcatattgtgatcaaattaaaaaaattaatttagaaattgagttctattttttatttattttaatttattttttaattttaaaaattttaattatttttatttagtttgattttaataaaaaaataaaaaccgaactaattagttataataatatattatttttaataatataaaaaaatagattataatgaaagttaaaatattttaattaaattttaaaatattaaaaataaaaaatttattaaaaaatcaaatcaaatcgaattaaattaaattaaattaaattaatttaatttttttaattttaaattaaactgaatatcagaaaaactaatctaatccataacaatataccgaaaaatataacattaacaaacggtcacttaaatcaaaatactaaaaagtaaaaagcacaataaatcactagaaggaaaatgttttataatatatcattttaattattataattttaaccattcataatatttaaatctcttcaattaaaattaataaaacaagtataatagtatattatttatgaatataaatttaatagaaagcaaagagtggaaatacattaattaagtttataaaaagtagataataaattgtcatttattttatgaaaactattttttaaggaaatatttctctttactttttaatatttgatattttgagaaaattattaataatttttaaattaaaaatttatttttaaataaaagaaatttaaaaaataactttcttatttattttctggtatttgacaatatagtaaattttattacataaattcattaatatttttattttttaaattaaaaaaagcctgagttatttttttttaaaaaaatttatatgcattttttaaaaaaaaaaataaaaccaaacttgataaatctacaaggtgcaagcgacgatagcaaaaatcgaattatatctttaaaaaaaaaaggggcaaaaatcgcaacctgaaaatcaaacctcttccccagcagacaaggttggctggtgattggtgaatgccccacttacaacaaactacacaaagcgctgttacacctaccgctgcctccatatctttttcttcacagtagcttttcgaaaaagaaagtaaaatcttcagagaaaaggtagagaaagagcggtaggagaatcttgtagacgcagaagaaatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaacagcaatgcgtccattgactgctgctgttacggcagcaggtggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgttcagaactcataagttgggttggggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgtagaaggggcggagatacactatgtctttataactttaaactctactgctgatcatgtctacatagaggaaataattaaaaaaagttgtaatttatgctgacagctggtgcctgctatttattaatggtcagaatcatttaaaatggtcttactgaggagaaaattaggtaagctagatgcatttaccttttttatttaagaagaatagtttgagtttgaaacatctttcaatttgttaattatttcccatatgtttagtaaaggcactcgtgttgttcagctgtcatgtcatgaattccttgcatatttttaaagattaaaaaaaaaacctgttagctccaatgttttgcttatatcaggcaatgcaatttgttttcctgtttagttcatttctcatttccttcttgaagggtaatatgttgaacttatcatcttatttctatgacaaccgttgagaatcgaacaatcacacaatctaaaaataagcatgaaaagaaattaacactctcaacggacaatcacacaatctaaaaataagcacgaaaagaaattaatacagatttaacgtggttcactaatttggctatgtccaggacggctagagttttcttatttctttatgagtttttggaaccgaataccctaagagaatattacatatttatatgtcaggaaaaatataaaaagacaaaatagtcctaaaaaaattcactttgggagctcccgcctcccacactccccgaccaacttagcgcggagttggattcgcgacagtaggacatggcgtatagtccataatatattattagtctccatattttaacaattcttccacttggagactaatccagtcaacccagtcattttcattctccatgccaatgtattcaacaaaaacacctaattgcaaatagctttcatactactgtaaaagtccaattgagaccttgcacaaccccagctttccagcatcaacacctttggtcaatatgtctagtcaatatgtctgctggatccttagtaccttcaatttttttttaaacacgtctcaccttcttccaccagactacgagtaaaatggtacctgcatcggatgtgttttgttttgaagtgatacactaaattcttcactaactgtatgacactctgactatcaataaaaaaaatcttgttaagctgcttcttgctcaattcttccaagtaatcggtcaatcatatcatttccttaccagcttcaacaattgcaacatattcagcctttgtagatgacatagagacacatttctgaagtttagacatccaactcaccgttgtcccatctatagtgtagacatacctagatgtgtttttccttgtatccatatctctactaagatcggcatcaacaaaaccttctaatattaccttgccattctcataacaaagtgatgtactagctgttcctttcaaatatctcagagaccacttcacagctttccaatactctttacctggatctgatatatatttgctcacagctcccactacatgtgctatatcaggtctggtgcagaccatagcatacatcaaactcccaactgcagaagcataaggtacttttgccatgtgatcacgctccatagttgtcttgggtgattgctcatttgacaatttgagatggctagccaaaggcgtgctcctggacttagtatcatcaattttgaacctggacaacaccttctgaatgtattgctcctgagataaatttagaattccagcagacctgtcccggaaaagcctcatccctaagaatatgctttgctgcatccaaatcttttatctcaaactctaatgacagtctccccttttgagttcgatcgaccatgtttaccatgcccacctttcaccttactcctgcctctaccctcgacactaagtggatctacgtgaactgctaattgcgaaaacaatagtatcccacgactcaggtagagaggataacaaaattagatttcttatctcatcatcaaaattaatcttgagagagcttaattgactgattatcatattaaattcgttaatatgatccgagatagattcacttttagacatctccaaattaatcaaacaatgcatcagatatattttgttaatggttgacggtttctcatatatgttcgctagacctgtcaacaatcgtacagttgttgtctccttaacgatattgaaggcaccgtttctcgccaacgtcaaatggatcaaccctagtgcctttcaatccttcaaatcccaaacctcctgtttcatagtttctggcttctctcctgacaagggttcatggaggttattctgatacaggtaatcttcaatctgcatctttcagaaaccaaaatctattccatcgaatttctcaattcgaatcttcgggctttccattgtcttttcagattgttgtgatacaaaccaaaagctctgataccagttgttaagaatcgaacagtcacacaatgtaaaaataagcacgaaaagaaattaacacataaatttaacgtggttcaccaatttagttacttccacggctactaagtttttttatttctctgagtttttgaaaccgaatacgctaggagaatatattattaggctccatatctcaacaataacttcatttttaacgaaattcatcatttttagtcatcatttcctataccttctttttcaggttaagctctctggttcaattagcagtcaatacttgactgctttgctcatggcagctcctttggctcttggagatgtagaaattgagattattgataaattgatatccattccttatgttgatatgactttgaagttaatggaacgattcggagtcactgcagaacacagtggtagctgggatcgtttcttcattcgtgggggtcaaaagtacaagtatgtttttttcaatatagatctgcaagctttttactgttgtttgactgaaattaacaacctgaaaaatcaattagatctcctggaaaagcttttgttgaaggtgatgcttcgagtgccagttatttcctggctggtgcagcaatcactggtggaaccatcactgttgaaggttgtggaacaagtagtttgcaggtatttcactcattaatatactgtcttactgcttcctataatcttaaaatggcttgtggaagtgcttattgtatccattgctttttgatgtttgaaaatttcgtcaaaattttctcaacatacacatgtatatatgatgtcttctccatggctccctactgtctaatatttcctaggaggaagtttgcaacataattgtatgcatttacacttaccaaaccaacttgcttagtttccttatctttatgaaaatcaagggggatgtaaggtttgctgaagtgcttgacaaaatgggagcaaaagtcacctggacagagaacagtgttactgtgactggaccaccacgcaattctcctattaaaaagcacttgcgagctattgatgtcaacatgaacaaaatgccagatgttgctatgacattggccgtggttgcactttttgctgatggccctactgccataagagacggtataatttgtccttcaaactttgtgaataccagtcatattctttcaataaaatgatcatgcagtacataaacaatgaactggtgattattatttggtggatcatagaacagctttggcttctattcatggaagatggcaatgaatgtaacccaattctaaatcattgaaatcccatttcttgggtccggacttcttgattaatacaaaatggtagtggctaaaagtaggaagcttcagctcatggaggatgaagtttctgaaggaaaaaatggtggctgtttcttttctttatctaataacagctagaacttccatatttgaccttcggtggatatttaagatctaaaacattgttatcttttcatctgaatcaaatcatcagcaggtttcacatatgtttccttgtaacttgtatgccaatgccaatctcttgatttctcttatttccagtggccagttggagagttaaagaaacagaacggatgattgcaatttgcacagagctcaggaaggtcagtttctggcagttcttaaactcatctgaatttcatgcaaacaaaaggtggtttatatttttttcttatataaatgacatttgggctgcaaatttcagctcggagcaaatgttgaagagggaccagactactgtttgatcactccacctgagaaactgaacatcacagagattgacacttatgatgatcacagaatggccatggcgttctcccttgctgcctgcggggatgttccagtcacaatcaaggaccctggttgcacaagaaaaacttttccagactactttgaggtccttgagaggttcactaagcatgattataaggatcacgatggcgactacaaggaccacgatattgactacaaagacgatgacgataaatgagccacacttcaacagtcacaattaataatagagagataggtttaccagcagtgtcactgcaactatgatgaagcaacctatatgtaataattttccccacctgattgcccattgtaatctttgataatttatgcgaggtgatgtttgttagtgtcccagccaaattgtccccaggtgactcgaagttacgtataggttatccagtgaatgcttggggtttagcgaatacttcattggtttctggaacgagacaatcgaaattggaaacacagtcgaccactcggtcataaaatgatttgatagtgaaagcattatttaaagtgatttgatagtggatcataaaatgttaacttagctaaccgttgataattattaaaacaattagtagttgaaagtacatagaatcatatagttccgggcctagttgcatcggccaatatatataaaatataaataagggaagtaattattcagaggtgcagacgttgtgcattatatttgtaacagcttgtgagatgggttggaggttggaggtggctccgggagatttatttctttgctttttcttcaaaatgtaaactaatgaacaagtgtttggggaggaaaaggagtacaagggactgtcaggactcagctatctacccggatcctctagagtcgacctgcaggcatgcaagcttggcgcgccattaaattaaactatgagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtactgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcgcgagctgttggctggctggtggcaggatatattgtggtgtaaacacgagctgttggctggctggtggcaggatatattgtggtgtaaacacgagctgttggctggctggtggcaggatatattgtggtgtaaacaaattgacgcttagacaacttaataacacattgcggacgtttttaatgtactgaatttacgccgaat

SEQ ID NO: 2 - DNA, 17523 bp, artificial
  T-DNA vector with wildtype cassava TME7 promoter driving G101A/A192T EPSPS
taatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaaaagctttcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtcgacagatctagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcgaattcgagctcggtacccggggatccgcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggaggcccgaaagtagtgaagcccagtctacgctatagtccgttttcttcctcgtcataatattcaatttgattaagaaaatggttctaataacgtcaatttttgaaaactttggtggattcaccaacttcattgatgtccaaaatgccaatttacatgacctgaaccgagcggtttgaataaacgggttgaatttgaacctagccatagcaggaagttcacaagataaggaatcaaatgatcgagacattggacgcatgattcggtttttaaaatcttactaaatcagcgttatcattaaagggtttaaatcacaactacacgtgctattcatttttttatattcagttatcatatctttatcatccatttaacatgaagtgtagattgctattaatattttatcaaactttaattattttattttattttaaatttaattaaaatatttttatatttataataacatataaatttatatgagtctttcaaaatcaaaacacttatacactattattgtaatctaaacaatcaccatagacttgagtcatattgtgatcaaattaaaaaaattaatttagaaattgagttctattttttatttattttaatttattttttaattttaaaaattttaattatttttatttagtttgattttaataaaaaaataaaaaccgaactaattagttataataatatattatttttaataatataaaaaaatagattataatgaaagttaaaatattttaattaaattttaaaatattaaaaataaaaaatttattaaaaaatcaaatcaaatcgaattaaattaaattaaattaaattaatttaatttttttaattttaaattaaactgaatatcagaaaaactaatctaatccataacaatataccgaaaaatataacattaacaaacggtcacttaaatcaaaatactaaaaagtaaaaagcacaataaatcactagaaggaaaatgttttataatatatcattttaattattataattttaaccattcataatatttaaatctcttcaattaaaattaataaaacaagtataatagtatattatttatgaatataaatttaatagaaagcaaagagtggaaatacattaattaagtttataaaaagtagataataaattgtcatttattttatgaaaactattttttaaggaaatatttctctttactttttaatatttgatattttgagaaaattattaataatttttaaattaaaaatttatttttaaataaaagaaatttaaaaaataactttcttatttattttctggtatttgacaatatagtaaattttattacataaattcattaatatttttattttttaaattaaaaaaagcctgagttatttttttttaaaaaaatttatatgcattttttaaaaaaaaaaataaaaccaaacttgataaatctacaaggtgcaagcgacgatagcaaaaatcgaattatatctttaaaaaaaaaaggggcaaaaatcgcaacctgaaaatcaaacctcttccccagcagacaaggttggctggtgattggtgaatgccccacttacaacaaactacacaaagcgctgttacacctaccgctgcctccatatctttttcttcacagtagcttttcgaaaaagaaagtaaaatcttcagagaaaaggtagagaaagagcggtaggagaatcttgtagacgcagaagaaatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcagctacagcaatgcgtccattgactgctgctgttacggcagcaggtggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgttcagaactcataagttgggttggggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgtagaaggggcggagatacactatgtctttataactttaaactctactgctgatcatgtctacatagaggaaataattaaaaaaagttgtaatttatgctgacagctggtgcctgctatttattaatggtcagaatcatttaaaatggtcttactgaggagaaaattaggtaagctagatgcatttaccttttttatttaagaagaatagtttgagtttgaaacatctttcaatttgttaattatttcccatatgtttagtaaaggcactcgtgttgttcagctgtcatgtcatgaattccttgcatatttttaaagattaaaaaaaaaacctgttagctccaatgttttgcttatatcaggcaatgcaatttgttttcctgtttagttcatttctcatttccttcttgaagggtaatatgttgaacttatcatcttatttctatgacaaccgttgagaatcgaacaatcacacaatctaaaaataagcatgaaaagaaattaacactctcaacggacaatcacacaatctaaaaataagcacgaaaagaaattaatacagatttaacgtggttcactaatttggctatgtccaggacggctagagttttcttatttctttatgagtttttggaaccgaataccctaagagaatattacatatttatatgtcaggaaaaatataaaaagacaaaatagtcctaaaaaaattcactttgggagctcccgcctcccacactccccgaccaacttagcgcggagttggattcgcgacagtaggacatggcgtatagtccataatatattattagtctccatattttaacaattcttccacttggagactaatccagtcaacccagtcattttcattctccatgccaatgtattcaacaaaaacacctaattgcaaatagctttcatactactgtaaaagtccaattgagaccttgcacaaccccagctttccagcatcaacacctttggtcaatatgtctagtcaatatgtctgctggatccttagtaccttcaatttttttttaaacacgtctcaccttcttccaccagactacgagtaaaatggtacctgcatcggatgtgttttgttttgaagtgatacactaaattcttcactaactgtatgacactctgactatcaataaaaaaaatcttgttaagctgcttcttgctcaattcttccaagtaatcggtcaatcatatcatttccttaccagcttcaacaattgcaacatattcagcctttgtagatgacatagagacacatttctgaagtttagacatccaactcaccgttgtcccatctatagtgtagacatacctagatgtgtttttccttgtatccatatctctactaagatcggcatcaacaaaaccttctaatattaccttgccattctcataacaaagtgatgtactagctgttcctttcaaatatctcagagaccacttcacagctttccaatactctttacctggatctgatatatatttgctcacagctcccactacatgtgctatatcaggtctggtgcagaccatagcatacatcaaactcccaactgcagaagcataaggtacttttgccatgtgatcacgctccatagttgtcttgggtgattgctcatttgacaatttgagatggctagccaaaggcgtgctcctggacttagtatcatcaattttgaacctggacaacaccttctgaatgtattgctcctgagataaatttagaattccagcagacctgtcccggaaaagcctcatccctaagaatatgctttgctgcatccaaatcttttatctcaaactctaatgacagtctccccttttgagttcgatcgaccatgtttaccatgcccacctttcaccttactcctgcctctaccctcgacactaagtggatctacgtgaactgctaattgcgaaaacaatagtatcccacgactcaggtagagaggataacaaaattagatttcttatctcatcatcaaaattaatcttgagagagcttaattgactgattatcatattaaattcgttaatatgatccgagatagattcacttttagacatctccaaattaatcaaacaatgcatcagatatattttgttaatggttgacggtttctcatatatgttcgctagacctgtcaacaatcgtacagttgttgtctccttaacgatattgaaggcaccgtttctcgccaacgtcaaatggatcaaccctagtgcctttcaatccttcaaatcccaaacctcctgtttcatagtttctggcttctctcctgacaagggttcatggaggttattctgatacaggtaatcttcaatctgcatctttcagaaaccaaaatctattccatcgaatttctcaattcgaatcttcgggctttccattgtcttttcagattgttgtgatacaaaccaaaagctctgataccagttgttaagaatcgaacagtcacacaatgtaaaaataagcacgaaaagaaattaacacataaatttaacgtggttcaccaatttagttacttccacggctactaagtttttttatttctctgagtttttgaaaccgaatacgctaggagaatatattattaggctccatatctcaacaataacttcatttttaacgaaattcatcatttttagtcatcatttcctataccttctttttcaggttaagctctctggttcaattagcagtcaatacttgactgctttgctcatggcagctcctttgactcttggagatgtagaaattgagattattgataaattgatatccattccttatgttgatatgactttgaagttaatggaacgattcggagtcactgcagaacacagtggtagctgggatcgtttcttcattcgtgggggtcaaaagtacaagtatgtttttttcaatatagatctgcaagctttttactgttgtttgactgaaattaacaacctgaaaaatcaattagatctcctggaaaagcttttgttgaaggtgatgcttcgagtgccagttatttcctggctggtgcagcaatcactggtggaaccatcactgttgaaggttgtggaacaagtagtttgcaggtatttcactcattaatatactgtcttactgcttcctataatcttaaaatggcttgtggaagtgcttattgtatccattgctttttgatgtttgaaaatttcgtcaaaattttctcaacatacacatgtatatatgatgtcttctccatggctccctactgtctaatatttcctaggaggaagtttgcaacataattgtatgcatttacacttaccaaaccaacttgcttagtttccttatctttatgaaaatcaagggggatgtaaggtttgctgaagtgcttgacaaaatgggagcaaaagtcacctggacagagaacagtgttactgtgactggaccaccacgcaattctcctattaaaaagcacttgcgagctattgatgtcaacatgaacaaaatgccagatgttgctatgacattggccgtggttgcactttttgctgatggccctactgccataagagacggtataatttgtccttcaaactttgtgaataccagtcatattctttcaataaaatgatcatgcagtacataaacaatgaactggtgattattatttggtggatcatagaacagctttggcttctattcatggaagatggcaatgaatgtaacccaattctaaatcattgaaatcccatttcttgggtccggacttcttgattaatacaaaatggtagtggctaaaagtaggaagcttcagctcatggaggatgaagtttctgaaggaaaaaatggtggctgtttcttttctttatctaataacagctagaacttccatatttgaccttcggtggatatttaagatctaaaacattgttatcttttcatctgaatcaaatcatcagcaggtttcacatatgtttccttgtaacttgtatgccaatgccaatctcttgatttctcttatttccagtggccagttggagagttaaagaaacagaacggatgattgcaatttgcacagagctcaggaaggtcagtttctggcagttcttaaactcatctgaatttcatgcaaacaaaaggtggtttatatttttttcttatataaatgacatttgggctgcaaatttcagctcggagcaaatgttgaagagggaccagactactgtttgatcactccacctgagaaactgaacatcacagagattgacacttatgatgatcacagaatggccatggcgttctcccttgctgcctgcggggatgttccagtcacaatcaaggaccctggttgcacaagaaaaacttttccagactactttgaggtccttgagaggttcactaagcatgattataaggatcacgatggcgactacaaggaccacgatattgactacaaagacgatgacgataaatgagccacacttcaacagtcacaattaataatagagagataggtttaccagcagtgtcactgcaactatgatgaagcaacctatatgtaataattttccccacctgattgcccattgtaatctttgataatttatgcgaggtgatgtttgttagtgtcccagccaaattgtccccaggtgactcgaagttacgtataggttatccagtgaatgcttggggtttagcgaatacttcattggtttctggaacgagacaatcgaaattggaaacacagtcgaccactcggtcataaaatgatttgatagtgaaagcattatttaaagtgatttgatagtggatcataaaatgttaacttagctaaccgttgataattattaaaacaattagtagttgaaagtacatagaatcatatagttccgggcctagttgcatcggccaatatatataaaatataaataagggaagtaattattcagaggtgcagacgttgtgcattatatttgtaacagcttgtgagatgggttggaggttggaggtggctccgggagatttatttctttgctttttcttcaaaatgtaaactaatgaacaagtgtttggggaggaaaaggagtacaagggactgtcaggactcagctatctacccggatcctctagagtcgacctgcaggcatgcaagcttggcgcgccattaaattaaactatgagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtactgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcgcgagctgttggctggctggtggcaggatatattgtggtgtaaacacgagctgttggctggctggtggcaggatatattgtggtgtaaacacgagctgttggctggctggtggcaggatatattgtggtgtaaacaaattgacgcttagacaacttaataacacattgcggacgtttttaatgtactgaatttacgccgaat

SEQ ID NO: 3 - DNA, 17523 bp, artificial
  T-DNA vector with wildtype cassava TME7 promoter driving T102I/P106A EPSPS
taatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaaaagctttcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtcgacagatctagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcgaattcgagctcggtacccggggatccgcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggaggcccgaaagtagtgaagcccagtctacgctatagtccgttttcttcctcgtcataatattcaatttgattaagaaaatggttctaataacgtcaatttttgaaaactttggtggattcaccaacttcattgatgtccaaaatgccaatttacatgacctgaaccgagcggtttgaataaacgggttgaatttgaacctagccatagcaggaagttcacaagataaggaatcaaatgatcgagacattggacgcatgattcggtttttaaaatcttactaaatcagcgttatcattaaagggtttaaatcacaactacacgtgctattcatttttttatattcagttatcatatctttatcatccatttaacatgaagtgtagattgctattaatattttatcaaactttaattattttattttattttaaatttaattaaaatatttttatatttataataacatataaatttatatgagtctttcaaaatcaaaacacttatacactattattgtaatctaaacaatcaccatagacttgagtcatattgtgatcaaattaaaaaaattaatttagaaattgagttctattttttatttattttaatttattttttaattttaaaaattttaattatttttatttagtttgattttaataaaaaaataaaaaccgaactaattagttataataatatattatttttaataatataaaaaaatagattataatgaaagttaaaatattttaattaaattttaaaatattaaaaataaaaaatttattaaaaaatcaaatcaaatcgaattaaattaaattaaattaaattaatttaatttttttaattttaaattaaactgaatatcagaaaaactaatctaatccataacaatataccgaaaaatataacattaacaaacggtcacttaaatcaaaatactaaaaagtaaaaagcacaataaatcactagaaggaaaatgttttataatatatcattttaattattataattttaaccattcataatatttaaatctcttcaattaaaattaataaaacaagtataatagtatattatttatgaatataaatttaatagaaagcaaagagtggaaatacattaattaagtttataaaaagtagataataaattgtcatttattttatgaaaactattttttaaggaaatatttctctttactttttaatatttgatattttgagaaaattattaataatttttaaattaaaaatttatttttaaataaaagaaatttaaaaaataactttcttatttattttctggtatttgacaatatagtaaattttattacataaattcattaatatttttattttttaaattaaaaaaagcctgagttatttttttttaaaaaaatttatatgcattttttaaaaaaaaaaataaaaccaaacttgataaatctacaaggtgcaagcgacgatagcaaaaatcgaattatatctttaaaaaaaaaaggggcaaaaatcgcaacctgaaaatcaaacctcttccccagcagacaaggttggctggtgattggtgaatgccccacttacaacaaactacacaaagcgctgttacacctaccgctgcctccatatctttttcttcacagtagcttttcgaaaaagaaagtaaaatcttcagagaaaaggtagagaaagagcggtaggagaatcttgtagacgcagaagaaatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtgctttgactgctgctgttacggcagcaggtggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgttcagaactcataagttgggttggggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgtagaaggggcggagatacactatgtctttataactttaaactctactgctgatcatgtctacatagaggaaataattaaaaaaagttgtaatttatgctgacagctggtgcctgctatttattaatggtcagaatcatttaaaatggtcttactgaggagaaaattaggtaagctagatgcatttaccttttttatttaagaagaatagtttgagtttgaaacatctttcaatttgttaattatttcccatatgtttagtaaaggcactcgtgttgttcagctgtcatgtcatgaattccttgcatatttttaaagattaaaaaaaaaacctgttagctccaatgttttgcttatatcaggcaatgcaatttgttttcctgtttagttcatttctcatttccttcttgaagggtaatatgttgaacttatcatcttatttctatgacaaccgttgagaatcgaacaatcacacaatctaaaaataagcatgaaaagaaattaacactctcaacggacaatcacacaatctaaaaataagcacgaaaagaaattaatacagatttaacgtggttcactaatttggctatgtccaggacggctagagttttcttatttctttatgagtttttggaaccgaataccctaagagaatattacatatttatatgtcaggaaaaatataaaaagacaaaatagtcctaaaaaaattcactttgggagctcccgcctcccacactccccgaccaacttagcgcggagttggattcgcgacagtaggacatggcgtatagtccataatatattattagtctccatattttaacaattcttccacttggagactaatccagtcaacccagtcattttcattctccatgccaatgtattcaacaaaaacacctaattgcaaatagctttcatactactgtaaaagtccaattgagaccttgcacaaccccagctttccagcatcaacacctttggtcaatatgtctagtcaatatgtctgctggatccttagtaccttcaatttttttttaaacacgtctcaccttcttccaccagactacgagtaaaatggtacctgcatcggatgtgttttgttttgaagtgatacactaaattcttcactaactgtatgacactctgactatcaataaaaaaaatcttgttaagctgcttcttgctcaattcttccaagtaatcggtcaatcatatcatttccttaccagcttcaacaattgcaacatattcagcctttgtagatgacatagagacacatttctgaagtttagacatccaactcaccgttgtcccatctatagtgtagacatacctagatgtgtttttccttgtatccatatctctactaagatcggcatcaacaaaaccttctaatattaccttgccattctcataacaaagtgatgtactagctgttcctttcaaatatctcagagaccacttcacagctttccaatactctttacctggatctgatatatatttgctcacagctcccactacatgtgctatatcaggtctggtgcagaccatagcatacatcaaactcccaactgcagaagcataaggtacttttgccatgtgatcacgctccatagttgtcttgggtgattgctcatttgacaatttgagatggctagccaaaggcgtgctcctggacttagtatcatcaattttgaacctggacaacaccttctgaatgtattgctcctgagataaatttagaattccagcagacctgtcccggaaaagcctcatccctaagaatatgctttgctgcatccaaatcttttatctcaaactctaatgacagtctccccttttgagttcgatcgaccatgtttaccatgcccacctttcaccttactcctgcctctaccctcgacactaagtggatctacgtgaactgctaattgcgaaaacaatagtatcccacgactcaggtagagaggataacaaaattagatttcttatctcatcatcaaaattaatcttgagagagcttaattgactgattatcatattaaattcgttaatatgatccgagatagattcacttttagacatctccaaattaatcaaacaatgcatcagatatattttgttaatggttgacggtttctcatatatgttcgctagacctgtcaacaatcgtacagttgttgtctccttaacgatattgaaggcaccgtttctcgccaacgtcaaatggatcaaccctagtgcctttcaatccttcaaatcccaaacctcctgtttcatagtttctggcttctctcctgacaagggttcatggaggttattctgatacaggtaatcttcaatctgcatctttcagaaaccaaaatctattccatcgaatttctcaattcgaatcttcgggctttccattgtcttttcagattgttgtgatacaaaccaaaagctctgataccagttgttaagaatcgaacagtcacacaatgtaaaaataagcacgaaaagaaattaacacataaatttaacgtggttcaccaatttagttacttccacggctactaagtttttttatttctctgagtttttgaaaccgaatacgctaggagaatatattattaggctccatatctcaacaataacttcatttttaacgaaattcatcatttttagtcatcatttcctataccttctttttcaggttaagctctctggttcaattagcagtcaatacttgactgctttgctcatggcagctcctttggctcttggagatgtagaaattgagattattgataaattgatatccattccttatgttgatatgactttgaagttaatggaacgattcggagtcactgcagaacacagtggtagctgggatcgtttcttcattcgtgggggtcaaaagtacaagtatgtttttttcaatatagatctgcaagctttttactgttgtttgactgaaattaacaacctgaaaaatcaattagatctcctggaaaagcttttgttgaaggtgatgcttcgagtgccagttatttcctggctggtgcagcaatcactggtggaaccatcactgttgaaggttgtggaacaagtagtttgcaggtatttcactcattaatatactgtcttactgcttcctataatcttaaaatggcttgtggaagtgcttattgtatccattgctttttgatgtttgaaaatttcgtcaaaattttctcaacatacacatgtatatatgatgtcttctccatggctccctactgtctaatatttcctaggaggaagtttgcaacataattgtatgcatttacacttaccaaaccaacttgcttagtttccttatctttatgaaaatcaagggggatgtaaggtttgctgaagtgcttgacaaaatgggagcaaaagtcacctggacagagaacagtgttactgtgactggaccaccacgcaattctcctattaaaaagcacttgcgagctattgatgtcaacatgaacaaaatgccagatgttgctatgacattggccgtggttgcactttttgctgatggccctactgccataagagacggtataatttgtccttcaaactttgtgaataccagtcatattctttcaataaaatgatcatgcagtacataaacaatgaactggtgattattatttggtggatcatagaacagctttggcttctattcatggaagatggcaatgaatgtaacccaattctaaatcattgaaatcccatttcttgggtccggacttcttgattaatacaaaatggtagtggctaaaagtaggaagcttcagctcatggaggatgaagtttctgaaggaaaaaatggtggctgtttcttttctttatctaataacagctagaacttccatatttgaccttcggtggatatttaagatctaaaacattgttatcttttcatctgaatcaaatcatcagcaggtttcacatatgtttccttgtaacttgtatgccaatgccaatctcttgatttctcttatttccagtggccagttggagagttaaagaaacagaacggatgattgcaatttgcacagagctcaggaaggtcagtttctggcagttcttaaactcatctgaatttcatgcaaacaaaaggtggtttatatttttttcttatataaatgacatttgggctgcaaatttcagctcggagcaaatgttgaagagggaccagactactgtttgatcactccacctgagaaactgaacatcacagagattgacacttatgatgatcacagaatggccatggcgttctcccttgctgcctgcggggatgttccagtcacaatcaaggaccctggttgcacaagaaaaacttttccagactactttgaggtccttgagaggttcactaagcatgattataaggatcacgatggcgactacaaggaccacgatattgactacaaagacgatgacgataaatgagccacacttcaacagtcacaattaataatagagagataggtttaccagcagtgtcactgcaactatgatgaagcaacctatatgtaataattttccccacctgattgcccattgtaatctttgataatttatgcgaggtgatgtttgttagtgtcccagccaaattgtccccaggtgactcgaagttacgtataggttatccagtgaatgcttggggtttagcgaatacttcattggtttctggaacgagacaatcgaaattggaaacacagtcgaccactcggtcataaaatgatttgatagtgaaagcattatttaaagtgatttgatagtggatcataaaatgttaacttagctaaccgttgataattattaaaacaattagtagttgaaagtacatagaatcatatagttccgggcctagttgcatcggccaatatatataaaatataaataagggaagtaattattcagaggtgcagacgttgtgcattatatttgtaacagcttgtgagatgggttggaggttggaggtggctccgggagatttatttctttgctttttcttcaaaatgtaaactaatgaacaagtgtttggggaggaaaaggagtacaagggactgtcaggactcagctatctacccggatcctctagagtcgacctgcaggcatgcaagcttggcgcgccattaaattaaactatgagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtactgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcgcgagctgttggctggctggtggcaggatatattgtggtgtaaacacgagctgttggctggctggtggcaggatatattgtggtgtaaacacgagctgttggctggctggtggcaggatatattgtggtgtaaacaaattgacgcttagacaacttaataacacattgcggacgtttttaatgtactgaatttacgccgaat

SEQ ID NO: 4 - DNA, 17523 bp, artificial
  T-DNA vector with wildtype cassava TME7 promoter driving T102I/P106I EPSPS
taatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaaaagctttcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtcgacagatctagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcgaattcgagctcggtacccggggatccgcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggaggcccgaaagtagtgaagcccagtctacgctatagtccgttttcttcctcgtcataatattcaatttgattaagaaaatggttctaataacgtcaatttttgaaaactttggtggattcaccaacttcattgatgtccaaaatgccaatttacatgacctgaaccgagcggtttgaataaacgggttgaatttgaacctagccatagcaggaagttcacaagataaggaatcaaatgatcgagacattggacgcatgattcggtttttaaaatcttactaaatcagcgttatcattaaagggtttaaatcacaactacacgtgctattcatttttttatattcagttatcatatctttatcatccatttaacatgaagtgtagattgctattaatattttatcaaactttaattattttattttattttaaatttaattaaaatatttttatatttataataacatataaatttatatgagtctttcaaaatcaaaacacttatacactattattgtaatctaaacaatcaccatagacttgagtcatattgtgatcaaattaaaaaaattaatttagaaattgagttctattttttatttattttaatttattttttaattttaaaaattttaattatttttatttagtttgattttaataaaaaaataaaaaccgaactaattagttataataatatattatttttaataatataaaaaaatagattataatgaaagttaaaatattttaattaaattttaaaatattaaaaataaaaaatttattaaaaaatcaaatcaaatcgaattaaattaaattaaattaaattaatttaatttttttaattttaaattaaactgaatatcagaaaaactaatctaatccataacaatataccgaaaaatataacattaacaaacggtcacttaaatcaaaatactaaaaagtaaaaagcacaataaatcactagaaggaaaatgttttataatatatcattttaattattataattttaaccattcataatatttaaatctcttcaattaaaattaataaaacaagtataatagtatattatttatgaatataaatttaatagaaagcaaagagtggaaatacattaattaagtttataaaaagtagataataaattgtcatttattttatgaaaactattttttaaggaaatatttctctttactttttaatatttgatattttgagaaaattattaataatttttaaattaaaaatttatttttaaataaaagaaatttaaaaaataactttcttatttattttctggtatttgacaatatagtaaattttattacataaattcattaatatttttattttttaaattaaaaaaagcctgagttatttttttttaaaaaaatttatatgcattttttaaaaaaaaaaataaaaccaaacttgataaatctacaaggtgcaagcgacgatagcaaaaatcgaattatatctttaaaaaaaaaaggggcaaaaatcgcaacctgaaaatcaaacctcttccccagcagacaaggttggctggtgattggtgaatgccccacttacaacaaactacacaaagcgctgttacacctaccgctgcctccatatctttttcttcacagtagcttttcgaaaaagaaagtaaaatcttcagagaaaaggtagagaaagagcggtaggagaatcttgtagacgcagaagaaatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtattttgactgctgctgttacggcagcaggtggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgttcagaactcataagttgggttggggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgtagaaggggcggagatacactatgtctttataactttaaactctactgctgatcatgtctacatagaggaaataattaaaaaaagttgtaatttatgctgacagctggtgcctgctatttattaatggtcagaatcatttaaaatggtcttactgaggagaaaattaggtaagctagatgcatttaccttttttatttaagaagaatagtttgagtttgaaacatctttcaatttgttaattatttcccatatgtttagtaaaggcactcgtgttgttcagctgtcatgtcatgaattccttgcatatttttaaagattaaaaaaaaaacctgttagctccaatgttttgcttatatcaggcaatgcaatttgttttcctgtttagttcatttctcatttccttcttgaagggtaatatgttgaacttatcatcttatttctatgacaaccgttgagaatcgaacaatcacacaatctaaaaataagcatgaaaagaaattaacactctcaacggacaatcacacaatctaaaaataagcacgaaaagaaattaatacagatttaacgtggttcactaatttggctatgtccaggacggctagagttttcttatttctttatgagtttttggaaccgaataccctaagagaatattacatatttatatgtcaggaaaaatataaaaagacaaaatagtcctaaaaaaattcactttgggagctcccgcctcccacactccccgaccaacttagcgcggagttggattcgcgacagtaggacatggcgtatagtccataatatattattagtctccatattttaacaattcttccacttggagactaatccagtcaacccagtcattttcattctccatgccaatgtattcaacaaaaacacctaattgcaaatagctttcatactactgtaaaagtccaattgagaccttgcacaaccccagctttccagcatcaacacctttggtcaatatgtctagtcaatatgtctgctggatccttagtaccttcaatttttttttaaacacgtctcaccttcttccaccagactacgagtaaaatggtacctgcatcggatgtgttttgttttgaagtgatacactaaattcttcactaactgtatgacactctgactatcaataaaaaaaatcttgttaagctgcttcttgctcaattcttccaagtaatcggtcaatcatatcatttccttaccagcttcaacaattgcaacatattcagcctttgtagatgacatagagacacatttctgaagtttagacatccaactcaccgttgtcccatctatagtgtagacatacctagatgtgtttttccttgtatccatatctctactaagatcggcatcaacaaaaccttctaatattaccttgccattctcataacaaagtgatgtactagctgttcctttcaaatatctcagagaccacttcacagctttccaatactctttacctggatctgatatatatttgctcacagctcccactacatgtgctatatcaggtctggtgcagaccatagcatacatcaaactcccaactgcagaagcataaggtacttttgccatgtgatcacgctccatagttgtcttgggtgattgctcatttgacaatttgagatggctagccaaaggcgtgctcctggacttagtatcatcaattttgaacctggacaacaccttctgaatgtattgctcctgagataaatttagaattccagcagacctgtcccggaaaagcctcatccctaagaatatgctttgctgcatccaaatcttttatctcaaactctaatgacagtctccccttttgagttcgatcgaccatgtttaccatgcccacctttcaccttactcctgcctctaccctcgacactaagtggatctacgtgaactgctaattgcgaaaacaatagtatcccacgactcaggtagagaggataacaaaattagatttcttatctcatcatcaaaattaatcttgagagagcttaattgactgattatcatattaaattcgttaatatgatccgagatagattcacttttagacatctccaaattaatcaaacaatgcatcagatatattttgttaatggttgacggtttctcatatatgttcgctagacctgtcaacaatcgtacagttgttgtctccttaacgatattgaaggcaccgtttctcgccaacgtcaaatggatcaaccctagtgcctttcaatccttcaaatcccaaacctcctgtttcatagtttctggcttctctcctgacaagggttcatggaggttattctgatacaggtaatcttcaatctgcatctttcagaaaccaaaatctattccatcgaatttctcaattcgaatcttcgggctttccattgtcttttcagattgttgtgatacaaaccaaaagctctgataccagttgttaagaatcgaacagtcacacaatgtaaaaataagcacgaaaagaaattaacacataaatttaacgtggttcaccaatttagttacttccacggctactaagtttttttatttctctgagtttttgaaaccgaatacgctaggagaatatattattaggctccatatctcaacaataacttcatttttaacgaaattcatcatttttagtcatcatttcctataccttctttttcaggttaagctctctggttcaattagcagtcaatacttgactgctttgctcatggcagctcctttggctcttggagatgtagaaattgagattattgataaattgatatccattccttatgttgatatgactttgaagttaatggaacgattcggagtcactgcagaacacagtggtagctgggatcgtttcttcattcgtgggggtcaaaagtacaagtatgtttttttcaatatagatctgcaagctttttactgttgtttgactgaaattaacaacctgaaaaatcaattagatctcctggaaaagcttttgttgaaggtgatgcttcgagtgccagttatttcctggctggtgcagcaatcactggtggaaccatcactgttgaaggttgtggaacaagtagtttgcaggtatttcactcattaatatactgtcttactgcttcctataatcttaaaatggcttgtggaagtgcttattgtatccattgctttttgatgtttgaaaatttcgtcaaaattttctcaacatacacatgtatatatgatgtcttctccatggctccctactgtctaatatttcctaggaggaagtttgcaacataattgtatgcatttacacttaccaaaccaacttgcttagtttccttatctttatgaaaatcaagggggatgtaaggtttgctgaagtgcttgacaaaatgggagcaaaagtcacctggacagagaacagtgttactgtgactggaccaccacgcaattctcctattaaaaagcacttgcgagctattgatgtcaacatgaacaaaatgccagatgttgctatgacattggccgtggttgcactttttgctgatggccctactgccataagagacggtataatttgtccttcaaactttgtgaataccagtcatattctttcaataaaatgatcatgcagtacataaacaatgaactggtgattattatttggtggatcatagaacagctttggcttctattcatggaagatggcaatgaatgtaacccaattctaaatcattgaaatcccatttcttgggtccggacttcttgattaatacaaaatggtagtggctaaaagtaggaagcttcagctcatggaggatgaagtttctgaaggaaaaaatggtggctgtttcttttctttatctaataacagctagaacttccatatttgaccttcggtggatatttaagatctaaaacattgttatcttttcatctgaatcaaatcatcagcaggtttcacatatgtttccttgtaacttgtatgccaatgccaatctcttgatttctcttatttccagtggccagttggagagttaaagaaacagaacggatgattgcaatttgcacagagctcaggaaggtcagtttctggcagttcttaaactcatctgaatttcatgcaaacaaaaggtggtttatatttttttcttatataaatgacatttgggctgcaaatttcagctcggagcaaatgttgaagagggaccagactactgtttgatcactccacctgagaaactgaacatcacagagattgacacttatgatgatcacagaatggccatggcgttctcccttgctgcctgcggggatgttccagtcacaatcaaggaccctggttgcacaagaaaaacttttccagactactttgaggtccttgagaggttcactaagcatgattataaggatcacgatggcgactacaaggaccacgatattgactacaaagacgatgacgataaatgagccacacttcaacagtcacaattaataatagagagataggtttaccagcagtgtcactgcaactatgatgaagcaacctatatgtaataattttccccacctgattgcccattgtaatctttgataatttatgcgaggtgatgtttgttagtgtcccagccaaattgtccccaggtgactcgaagttacgtataggttatccagtgaatgcttggggtttagcgaatacttcattggtttctggaacgagacaatcgaaattggaaacacagtcgaccactcggtcataaaatgatttgatagtgaaagcattatttaaagtgatttgatagtggatcataaaatgttaacttagctaaccgttgataattattaaaacaattagtagttgaaagtacatagaatcatatagttccgggcctagttgcatcggccaatatatataaaatataaataagggaagtaattattcagaggtgcagacgttgtgcattatatttgtaacagcttgtgagatgggttggaggttggaggtggctccgggagatttatttctttgctttttcttcaaaatgtaaactaatgaacaagtgtttggggaggaaaaggagtacaagggactgtcaggactcagctatctacccggatcctctagagtcgacctgcaggcatgcaagcttggcgcgccattaaattaaactatgagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtactgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcgcgagctgttggctggctggtggcaggatatattgtggtgtaaacacgagctgttggctggctggtggcaggatatattgtggtgtaaacacgagctgttggctggctggtggcaggatatattgtggtgtaaacaaattgacgcttagacaacttaataacacattgcggacgtttttaatgtactgaatttacgccgaat

SEQ ID NO: 5 - DNA, 16656 bp, artificial
  T-DNA vector with double 35s promoter driving wildtype EPSPS
taatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaaaagctttcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtcgacagatctagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcgaattcgagctcggtacccggggatccctgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatctgtcgatcgaccatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaacagcaatgcgtccattgactgctgctgttacggcagcaggtggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgttcagaactcataagttgggttggggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgtagaaggggcggagatacactatgtctttataactttaaactctactgctgatcatgtctacatagaggaaataattaaaaaaagttgtaatttatgctgacagctggtgcctgctatttattaatggtcagaatcatttaaaatggtcttactgaggagaaaattaggtaagctagatgcatttaccttttttatttaagaagaatagtttgagtttgaaacatctttcaatttgttaattatttcccatatgtttagtaaaggcactcgtgttgttcagctgtcatgtcatgaattccttgcatatttttaaagattaaaaaaaaaacctgttagctccaatgttttgcttatatcaggcaatgcaatttgttttcctgtttagttcatttctcatttccttcttgaagggtaatatgttgaacttatcatcttatttctatgacaaccgttgagaatcgaacaatcacacaatctaaaaataagcatgaaaagaaattaacactctcaacggacaatcacacaatctaaaaataagcacgaaaagaaattaatacagatttaacgtggttcactaatttggctatgtccaggacggctagagttttcttatttctttatgagtttttggaaccgaataccctaagagaatattacatatttatatgtcaggaaaaatataaaaagacaaaatagtcctaaaaaaattcactttgggagctcccgcctcccacactccccgaccaacttagcgcggagttggattcgcgacagtaggacatggcgtatagtccataatatattattagtctccatattttaacaattcttccacttggagactaatccagtcaacccagtcattttcattctccatgccaatgtattcaacaaaaacacctaattgcaaatagctttcatactactgtaaaagtccaattgagaccttgcacaaccccagctttccagcatcaacacctttggtcaatatgtctagtcaatatgtctgctggatccttagtaccttcaatttttttttaaacacgtctcaccttcttccaccagactacgagtaaaatggtacctgcatcggatgtgttttgttttgaagtgatacactaaattcttcactaactgtatgacactctgactatcaataaaaaaaatcttgttaagctgcttcttgctcaattcttccaagtaatcggtcaatcatatcatttccttaccagcttcaacaattgcaacatattcagcctttgtagatgacatagagacacatttctgaagtttagacatccaactcaccgttgtcccatctatagtgtagacatacctagatgtgtttttccttgtatccatatctctactaagatcggcatcaacaaaaccttctaatattaccttgccattctcataacaaagtgatgtactagctgttcctttcaaatatctcagagaccacttcacagctttccaatactctttacctggatctgatatatatttgctcacagctcccactacatgtgctatatcaggtctggtgcagaccatagcatacatcaaactcccaactgcagaagcataaggtacttttgccatgtgatcacgctccatagttgtcttgggtgattgctcatttgacaatttgagatggctagccaaaggcgtgctcctggacttagtatcatcaattttgaacctggacaacaccttctgaatgtattgctcctgagataaatttagaattccagcagacctgtcccggaaaagcctcatccctaagaatatgctttgctgcatccaaatcttttatctcaaactctaatgacagtctccccttttgagttcgatcgaccatgtttaccatgcccacctttcaccttactcctgcctctaccctcgacactaagtggatctacgtgaactgctaattgcgaaaacaatagtatcccacgactcaggtagagaggataacaaaattagatttcttatctcatcatcaaaattaatcttgagagagcttaattgactgattatcatattaaattcgttaatatgatccgagatagattcacttttagacatctccaaattaatcaaacaatgcatcagatatattttgttaatggttgacggtttctcatatatgttcgctagacctgtcaacaatcgtacagttgttgtctccttaacgatattgaaggcaccgtttctcgccaacgtcaaatggatcaaccctagtgcctttcaatccttcaaatcccaaacctcctgtttcatagtttctggcttctctcctgacaagggttcatggaggttattctgatacaggtaatcttcaatctgcatctttcagaaaccaaaatctattccatcgaatttctcaattcgaatcttcgggctttccattgtcttttcagattgttgtgatacaaaccaaaagctctgataccagttgttaagaatcgaacagtcacacaatgtaaaaataagcacgaaaagaaattaacacataaatttaacgtggttcaccaatttagttacttccacggctactaagtttttttatttctctgagtttttgaaaccgaatacgctaggagaatatattattaggctccatatctcaacaataacttcatttttaacgaaattcatcatttttagtcatcatttcctataccttctttttcaggttaagctctctggttcaattagcagtcaatacttgactgctttgctcatggcagctcctttggctcttggagatgtagaaattgagattattgataaattgatatccattccttatgttgatatgactttgaagttaatggaacgattcggagtcactgcagaacacagtggtagctgggatcgtttcttcattcgtgggggtcaaaagtacaagtatgtttttttcaatatagatctgcaagctttttactgttgtttgactgaaattaacaacctgaaaaatcaattagatctcctggaaaagcttttgttgaaggtgatgcttcgagtgccagttatttcctggctggtgcagcaatcactggtggaaccatcactgttgaaggttgtggaacaagtagtttgcaggtatttcactcattaatatactgtcttactgcttcctataatcttaaaatggcttgtggaagtgcttattgtatccattgctttttgatgtttgaaaatttcgtcaaaattttctcaacatacacatgtatatatgatgtcttctccatggctccctactgtctaatatttcctaggaggaagtttgcaacataattgtatgcatttacacttaccaaaccaacttgcttagtttccttatctttatgaaaatcaagggggatgtaaggtttgctgaagtgcttgacaaaatgggagcaaaagtcacctggacagagaacagtgttactgtgactggaccaccacgcaattctcctattaaaaagcacttgcgagctattgatgtcaacatgaacaaaatgccagatgttgctatgacattggccgtggttgcactttttgctgatggccctactgccataagagacggtataatttgtccttcaaactttgtgaataccagtcatattctttcaataaaatgatcatgcagtacataaacaatgaactggtgattattatttggtggatcatagaacagctttggcttctattcatggaagatggcaatgaatgtaacccaattctaaatcattgaaatcccatttcttgggtccggacttcttgattaatacaaaatggtagtggctaaaagtaggaagcttcagctcatggaggatgaagtttctgaaggaaaaaatggtggctgtttcttttctttatctaataacagctagaacttccatatttgaccttcggtggatatttaagatctaaaacattgttatcttttcatctgaatcaaatcatcagcaggtttcacatatgtttccttgtaacttgtatgccaatgccaatctcttgatttctcttatttccagtggccagttggagagttaaagaaacagaacggatgattgcaatttgcacagagctcaggaaggtcagtttctggcagttcttaaactcatctgaatttcatgcaaacaaaaggtggtttatatttttttcttatataaatgacatttgggctgcaaatttcagctcggagcaaatgttgaagagggaccagactactgtttgatcactccacctgagaaactgaacatcacagagattgacacttatgatgatcacagaatggccatggcgttctcccttgctgcctgcggggatgttccagtcacaatcaaggaccctggttgcacaagaaaaacttttccagactactttgaggtccttgagaggttcactaagcatgattataaggatcacgatggcgactacaaggaccacgatattgactacaaagacgatgacgataaatgagccacacttcaacagtcacaattaataatagagagataggtttaccagcagtgtcactgcaactatgatgaagcaacctatatgtaataattttccccacctgattgcccattgtaatctttgataatttatgcgaggtgatgtttgttagtgtcccagccaaattgtccccaggtgactcgaagttacgtataggttatccagtgaatgcttggggtttagcgaatacttcattggtttctggaacgagacaatcgaaattggaaacacagtcgaccactcggtcataaaatgatttgatagtgaaagcattatttaaagtgatttgatagtggatcataaaatgttaacttagctaaccgttgataattattaaaacaattagtagttgaaagtacatagaatcatatagttccgggcctagttgcatcggccaatatatataaaatataaataagggaagtaattattcagaggtgcagacgttgtgcattatatttgtaacagcttgtgagatgggttggaggttggaggtggctccgggagatttatttctttgctttttcttcaaaatgtaaactaatgaacaagtgtttggggaggaaaaggagtacaagggactgtcaggactcagctatctacccggatcctctagagtcgacctgcaggcatgcaagcttggcgcgccattaaattaaactatgagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtactgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcgcgagctgttggctggctggtggcaggatatattgtggtgtaaacacgagctgttggctggctggtggcaggatatattgtggtgtaaacacgagctgttggctggctggtggcaggatatattgtggtgtaaacaaattgacgcttagacaacttaataacacattgcggacgtttttaatgtactgaatttacgccgaat

SEQ ID NO: 6 - DNA, 16656 bp, artificial
  T-DNA vector with double 35s promoter driving G101A/A192T EPSPS
taatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaaaagctttcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtcgacagatctagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcgaattcgagctcggtacccggggatccctgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatctgtcgatcgaccatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcagctacagcaatgcgtccattgactgctgctgttacggcagcaggtggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgttcagaactcataagttgggttggggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgtagaaggggcggagatacactatgtctttataactttaaactctactgctgatcatgtctacatagaggaaataattaaaaaaagttgtaatttatgctgacagctggtgcctgctatttattaatggtcagaatcatttaaaatggtcttactgaggagaaaattaggtaagctagatgcatttaccttttttatttaagaagaatagtttgagtttgaaacatctttcaatttgttaattatttcccatatgtttagtaaaggcactcgtgttgttcagctgtcatgtcatgaattccttgcatatttttaaagattaaaaaaaaaacctgttagctccaatgttttgcttatatcaggcaatgcaatttgttttcctgtttagttcatttctcatttccttcttgaagggtaatatgttgaacttatcatcttatttctatgacaaccgttgagaatcgaacaatcacacaatctaaaaataagcatgaaaagaaattaacactctcaacggacaatcacacaatctaaaaataagcacgaaaagaaattaatacagatttaacgtggttcactaatttggctatgtccaggacggctagagttttcttatttctttatgagtttttggaaccgaataccctaagagaatattacatatttatatgtcaggaaaaatataaaaagacaaaatagtcctaaaaaaattcactttgggagctcccgcctcccacactccccgaccaacttagcgcggagttggattcgcgacagtaggacatggcgtatagtccataatatattattagtctccatattttaacaattcttccacttggagactaatccagtcaacccagtcattttcattctccatgccaatgtattcaacaaaaacacctaattgcaaatagctttcatactactgtaaaagtccaattgagaccttgcacaaccccagctttccagcatcaacacctttggtcaatatgtctagtcaatatgtctgctggatccttagtaccttcaatttttttttaaacacgtctcaccttcttccaccagactacgagtaaaatggtacctgcatcggatgtgttttgttttgaagtgatacactaaattcttcactaactgtatgacactctgactatcaataaaaaaaatcttgttaagctgcttcttgctcaattcttccaagtaatcggtcaatcatatcatttccttaccagcttcaacaattgcaacatattcagcctttgtagatgacatagagacacatttctgaagtttagacatccaactcaccgttgtcccatctatagtgtagacatacctagatgtgtttttccttgtatccatatctctactaagatcggcatcaacaaaaccttctaatattaccttgccattctcataacaaagtgatgtactagctgttcctttcaaatatctcagagaccacttcacagctttccaatactctttacctggatctgatatatatttgctcacagctcccactacatgtgctatatcaggtctggtgcagaccatagcatacatcaaactcccaactgcagaagcataaggtacttttgccatgtgatcacgctccatagttgtcttgggtgattgctcatttgacaatttgagatggctagccaaaggcgtgctcctggacttagtatcatcaattttgaacctggacaacaccttctgaatgtattgctcctgagataaatttagaattccagcagacctgtcccggaaaagcctcatccctaagaatatgctttgctgcatccaaatcttttatctcaaactctaatgacagtctccccttttgagttcgatcgaccatgtttaccatgcccacctttcaccttactcctgcctctaccctcgacactaagtggatctacgtgaactgctaattgcgaaaacaatagtatcccacgactcaggtagagaggataacaaaattagatttcttatctcatcatcaaaattaatcttgagagagcttaattgactgattatcatattaaattcgttaatatgatccgagatagattcacttttagacatctccaaattaatcaaacaatgcatcagatatattttgttaatggttgacggtttctcatatatgttcgctagacctgtcaacaatcgtacagttgttgtctccttaacgatattgaaggcaccgtttctcgccaacgtcaaatggatcaaccctagtgcctttcaatccttcaaatcccaaacctcctgtttcatagtttctggcttctctcctgacaagggttcatggaggttattctgatacaggtaatcttcaatctgcatctttcagaaaccaaaatctattccatcgaatttctcaattcgaatcttcgggctttccattgtcttttcagattgttgtgatacaaaccaaaagctctgataccagttgttaagaatcgaacagtcacacaatgtaaaaataagcacgaaaagaaattaacacataaatttaacgtggttcaccaatttagttacttccacggctactaagtttttttatttctctgagtttttgaaaccgaatacgctaggagaatatattattaggctccatatctcaacaataacttcatttttaacgaaattcatcatttttagtcatcatttcctataccttctttttcaggttaagctctctggttcaattagcagtcaatacttgactgctttgctcatggcagctcctttgactcttggagatgtagaaattgagattattgataaattgatatccattccttatgttgatatgactttgaagttaatggaacgattcggagtcactgcagaacacagtggtagctgggatcgtttcttcattcgtgggggtcaaaagtacaagtatgtttttttcaatatagatctgcaagctttttactgttgtttgactgaaattaacaacctgaaaaatcaattagatctcctggaaaagcttttgttgaaggtgatgcttcgagtgccagttatttcctggctggtgcagcaatcactggtggaaccatcactgttgaaggttgtggaacaagtagtttgcaggtatttcactcattaatatactgtcttactgcttcctataatcttaaaatggcttgtggaagtgcttattgtatccattgctttttgatgtttgaaaatttcgtcaaaattttctcaacatacacatgtatatatgatgtcttctccatggctccctactgtctaatatttcctaggaggaagtttgcaacataattgtatgcatttacacttaccaaaccaacttgcttagtttccttatctttatgaaaatcaagggggatgtaaggtttgctgaagtgcttgacaaaatgggagcaaaagtcacctggacagagaacagtgttactgtgactggaccaccacgcaattctcctattaaaaagcacttgcgagctattgatgtcaacatgaacaaaatgccagatgttgctatgacattggccgtggttgcactttttgctgatggccctactgccataagagacggtataatttgtccttcaaactttgtgaataccagtcatattctttcaataaaatgatcatgcagtacataaacaatgaactggtgattattatttggtggatcatagaacagctttggcttctattcatggaagatggcaatgaatgtaacccaattctaaatcattgaaatcccatttcttgggtccggacttcttgattaatacaaaatggtagtggctaaaagtaggaagcttcagctcatggaggatgaagtttctgaaggaaaaaatggtggctgtttcttttctttatctaataacagctagaacttccatatttgaccttcggtggatatttaagatctaaaacattgttatcttttcatctgaatcaaatcatcagcaggtttcacatatgtttccttgtaacttgtatgccaatgccaatctcttgatttctcttatttccagtggccagttggagagttaaagaaacagaacggatgattgcaatttgcacagagctcaggaaggtcagtttctggcagttcttaaactcatctgaatttcatgcaaacaaaaggtggtttatatttttttcttatataaatgacatttgggctgcaaatttcagctcggagcaaatgttgaagagggaccagactactgtttgatcactccacctgagaaactgaacatcacagagattgacacttatgatgatcacagaatggccatggcgttctcccttgctgcctgcggggatgttccagtcacaatcaaggaccctggttgcacaagaaaaacttttccagactactttgaggtccttgagaggttcactaagcatgattataaggatcacgatggcgactacaaggaccacgatattgactacaaagacgatgacgataaatgagccacacttcaacagtcacaattaataatagagagataggtttaccagcagtgtcactgcaactatgatgaagcaacctatatgtaataattttccccacctgattgcccattgtaatctttgataatttatgcgaggtgatgtttgttagtgtcccagccaaattgtccccaggtgactcgaagttacgtataggttatccagtgaatgcttggggtttagcgaatacttcattggtttctggaacgagacaatcgaaattggaaacacagtcgaccactcggtcataaaatgatttgatagtgaaagcattatttaaagtgatttgatagtggatcataaaatgttaacttagctaaccgttgataattattaaaacaattagtagttgaaagtacatagaatcatatagttccgggcctagttgcatcggccaatatatataaaatataaataagggaagtaattattcagaggtgcagacgttgtgcattatatttgtaacagcttgtgagatgggttggaggttggaggtggctccgggagatttatttctttgctttttcttcaaaatgtaaactaatgaacaagtgtttggggaggaaaaggagtacaagggactgtcaggactcagctatctacccggatcctctagagtcgacctgcaggcatgcaagcttggcgcgccattaaattaaactatgagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtactgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcgcgagctgttggctggctggtggcaggatatattgtggtgtaaacacgagctgttggctggctggtggcaggatatattgtggtgtaaacacgagctgttggctggctggtggcaggatatattgtggtgtaaacaaattgacgcttagacaacttaataacacattgcggacgtttttaatgtactgaatttacgccgaat

SEQ ID NO: 7 - DNA, 16656 bp, artificial
  T-DNA vector with double 35s promoter driving T102I/P106A EPSPS
taatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaaaagctttcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtcgacagatctagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcgaattcgagctcggtacccggggatccctgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatctgtcgatcgaccatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtgctttgactgctgctgttacggcagcaggtggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgttcagaactcataagttgggttggggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgtagaaggggcggagatacactatgtctttataactttaaactctactgctgatcatgtctacatagaggaaataattaaaaaaagttgtaatttatgctgacagctggtgcctgctatttattaatggtcagaatcatttaaaatggtcttactgaggagaaaattaggtaagctagatgcatttaccttttttatttaagaagaatagtttgagtttgaaacatctttcaatttgttaattatttcccatatgtttagtaaaggcactcgtgttgttcagctgtcatgtcatgaattccttgcatatttttaaagattaaaaaaaaaacctgttagctccaatgttttgcttatatcaggcaatgcaatttgttttcctgtttagttcatttctcatttccttcttgaagggtaatatgttgaacttatcatcttatttctatgacaaccgttgagaatcgaacaatcacacaatctaaaaataagcatgaaaagaaattaacactctcaacggacaatcacacaatctaaaaataagcacgaaaagaaattaatacagatttaacgtggttcactaatttggctatgtccaggacggctagagttttcttatttctttatgagtttttggaaccgaataccctaagagaatattacatatttatatgtcaggaaaaatataaaaagacaaaatagtcctaaaaaaattcactttgggagctcccgcctcccacactccccgaccaacttagcgcggagttggattcgcgacagtaggacatggcgtatagtccataatatattattagtctccatattttaacaattcttccacttggagactaatccagtcaacccagtcattttcattctccatgccaatgtattcaacaaaaacacctaattgcaaatagctttcatactactgtaaaagtccaattgagaccttgcacaaccccagctttccagcatcaacacctttggtcaatatgtctagtcaatatgtctgctggatccttagtaccttcaatttttttttaaacacgtctcaccttcttccaccagactacgagtaaaatggtacctgcatcggatgtgttttgttttgaagtgatacactaaattcttcactaactgtatgacactctgactatcaataaaaaaaatcttgttaagctgcttcttgctcaattcttccaagtaatcggtcaatcatatcatttccttaccagcttcaacaattgcaacatattcagcctttgtagatgacatagagacacatttctgaagtttagacatccaactcaccgttgtcccatctatagtgtagacatacctagatgtgtttttccttgtatccatatctctactaagatcggcatcaacaaaaccttctaatattaccttgccattctcataacaaagtgatgtactagctgttcctttcaaatatctcagagaccacttcacagctttccaatactctttacctggatctgatatatatttgctcacagctcccactacatgtgctatatcaggtctggtgcagaccatagcatacatcaaactcccaactgcagaagcataaggtacttttgccatgtgatcacgctccatagttgtcttgggtgattgctcatttgacaatttgagatggctagccaaaggcgtgctcctggacttagtatcatcaattttgaacctggacaacaccttctgaatgtattgctcctgagataaatttagaattccagcagacctgtcccggaaaagcctcatccctaagaatatgctttgctgcatccaaatcttttatctcaaactctaatgacagtctccccttttgagttcgatcgaccatgtttaccatgcccacctttcaccttactcctgcctctaccctcgacactaagtggatctacgtgaactgctaattgcgaaaacaatagtatcccacgactcaggtagagaggataacaaaattagatttcttatctcatcatcaaaattaatcttgagagagcttaattgactgattatcatattaaattcgttaatatgatccgagatagattcacttttagacatctccaaattaatcaaacaatgcatcagatatattttgttaatggttgacggtttctcatatatgttcgctagacctgtcaacaatcgtacagttgttgtctccttaacgatattgaaggcaccgtttctcgccaacgtcaaatggatcaaccctagtgcctttcaatccttcaaatcccaaacctcctgtttcatagtttctggcttctctcctgacaagggttcatggaggttattctgatacaggtaatcttcaatctgcatctttcagaaaccaaaatctattccatcgaatttctcaattcgaatcttcgggctttccattgtcttttcagattgttgtgatacaaaccaaaagctctgataccagttgttaagaatcgaacagtcacacaatgtaaaaataagcacgaaaagaaattaacacataaatttaacgtggttcaccaatttagttacttccacggctactaagtttttttatttctctgagtttttgaaaccgaatacgctaggagaatatattattaggctccatatctcaacaataacttcatttttaacgaaattcatcatttttagtcatcatttcctataccttctttttcaggttaagctctctggttcaattagcagtcaatacttgactgctttgctcatggcagctcctttggctcttggagatgtagaaattgagattattgataaattgatatccattccttatgttgatatgactttgaagttaatggaacgattcggagtcactgcagaacacagtggtagctgggatcgtttcttcattcgtgggggtcaaaagtacaagtatgtttttttcaatatagatctgcaagctttttactgttgtttgactgaaattaacaacctgaaaaatcaattagatctcctggaaaagcttttgttgaaggtgatgcttcgagtgccagttatttcctggctggtgcagcaatcactggtggaaccatcactgttgaaggttgtggaacaagtagtttgcaggtatttcactcattaatatactgtcttactgcttcctataatcttaaaatggcttgtggaagtgcttattgtatccattgctttttgatgtttgaaaatttcgtcaaaattttctcaacatacacatgtatatatgatgtcttctccatggctccctactgtctaatatttcctaggaggaagtttgcaacataattgtatgcatttacacttaccaaaccaacttgcttagtttccttatctttatgaaaatcaagggggatgtaaggtttgctgaagtgcttgacaaaatgggagcaaaagtcacctggacagagaacagtgttactgtgactggaccaccacgcaattctcctattaaaaagcacttgcgagctattgatgtcaacatgaacaaaatgccagatgttgctatgacattggccgtggttgcactttttgctgatggccctactgccataagagacggtataatttgtccttcaaactttgtgaataccagtcatattctttcaataaaatgatcatgcagtacataaacaatgaactggtgattattatttggtggatcatagaacagctttggcttctattcatggaagatggcaatgaatgtaacccaattctaaatcattgaaatcccatttcttgggtccggacttcttgattaatacaaaatggtagtggctaaaagtaggaagcttcagctcatggaggatgaagtttctgaaggaaaaaatggtggctgtttcttttctttatctaataacagctagaacttccatatttgaccttcggtggatatttaagatctaaaacattgttatcttttcatctgaatcaaatcatcagcaggtttcacatatgtttccttgtaacttgtatgccaatgccaatctcttgatttctcttatttccagtggccagttggagagttaaagaaacagaacggatgattgcaatttgcacagagctcaggaaggtcagtttctggcagttcttaaactcatctgaatttcatgcaaacaaaaggtggtttatatttttttcttatataaatgacatttgggctgcaaatttcagctcggagcaaatgttgaagagggaccagactactgtttgatcactccacctgagaaactgaacatcacagagattgacacttatgatgatcacagaatggccatggcgttctcccttgctgcctgcggggatgttccagtcacaatcaaggaccctggttgcacaagaaaaacttttccagactactttgaggtccttgagaggttcactaagcatgattataaggatcacgatggcgactacaaggaccacgatattgactacaaagacgatgacgataaatgagccacacttcaacagtcacaattaataatagagagataggtttaccagcagtgtcactgcaactatgatgaagcaacctatatgtaataattttccccacctgattgcccattgtaatctttgataatttatgcgaggtgatgtttgttagtgtcccagccaaattgtccccaggtgactcgaagttacgtataggttatccagtgaatgcttggggtttagcgaatacttcattggtttctggaacgagacaatcgaaattggaaacacagtcgaccactcggtcataaaatgatttgatagtgaaagcattatttaaagtgatttgatagtggatcataaaatgttaacttagctaaccgttgataattattaaaacaattagtagttgaaagtacatagaatcatatagttccgggcctagttgcatcggccaatatatataaaatataaataagggaagtaattattcagaggtgcagacgttgtgcattatatttgtaacagcttgtgagatgggttggaggttggaggtggctccgggagatttatttctttgctttttcttcaaaatgtaaactaatgaacaagtgtttggggaggaaaaggagtacaagggactgtcaggactcagctatctacccggatcctctagagtcgacctgcaggcatgcaagcttggcgcgccattaaattaaactatgagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtactgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcgcgagctgttggctggctggtggcaggatatattgtggtgtaaacacgagctgttggctggctggtggcaggatatattgtggtgtaaacacgagctgttggctggctggtggcaggatatattgtggtgtaaacaaattgacgcttagacaacttaataacacattgcggacgtttttaatgtactgaatttacgccgaat

SEQ ID NO: 8 - DNA, 16656 bp, artificial
  T-DNA vector with double 35s promoter driving T102I/P106I EPSPS
taatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaaaagctttcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtcgacagatctagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcgaattcgagctcggtacccggggatccctgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgactctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatctgtcgatcgaccatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtattttgactgctgctgttacggcagcaggtggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgttcagaactcataagttgggttggggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgtagaaggggcggagatacactatgtctttataactttaaactctactgctgatcatgtctacatagaggaaataattaaaaaaagttgtaatttatgctgacagctggtgcctgctatttattaatggtcagaatcatttaaaatggtcttactgaggagaaaattaggtaagctagatgcatttaccttttttatttaagaagaatagtttgagtttgaaacatctttcaatttgttaattatttcccatatgtttagtaaaggcactcgtgttgttcagctgtcatgtcatgaattccttgcatatttttaaagattaaaaaaaaaacctgttagctccaatgttttgcttatatcaggcaatgcaatttgttttcctgtttagttcatttctcatttccttcttgaagggtaatatgttgaacttatcatcttatttctatgacaaccgttgagaatcgaacaatcacacaatctaaaaataagcatgaaaagaaattaacactctcaacggacaatcacacaatctaaaaataagcacgaaaagaaattaatacagatttaacgtggttcactaatttggctatgtccaggacggctagagttttcttatttctttatgagtttttggaaccgaataccctaagagaatattacatatttatatgtcaggaaaaatataaaaagacaaaatagtcctaaaaaaattcactttgggagctcccgcctcccacactccccgaccaacttagcgcggagttggattcgcgacagtaggacatggcgtatagtccataatatattattagtctccatattttaacaattcttccacttggagactaatccagtcaacccagtcattttcattctccatgccaatgtattcaacaaaaacacctaattgcaaatagctttcatactactgtaaaagtccaattgagaccttgcacaaccccagctttccagcatcaacacctttggtcaatatgtctagtcaatatgtctgctggatccttagtaccttcaatttttttttaaacacgtctcaccttcttccaccagactacgagtaaaatggtacctgcatcggatgtgttttgttttgaagtgatacactaaattcttcactaactgtatgacactctgactatcaataaaaaaaatcttgttaagctgcttcttgctcaattcttccaagtaatcggtcaatcatatcatttccttaccagcttcaacaattgcaacatattcagcctttgtagatgacatagagacacatttctgaagtttagacatccaactcaccgttgtcccatctatagtgtagacatacctagatgtgtttttccttgtatccatatctctactaagatcggcatcaacaaaaccttctaatattaccttgccattctcataacaaagtgatgtactagctgttcctttcaaatatctcagagaccacttcacagctttccaatactctttacctggatctgatatatatttgctcacagctcccactacatgtgctatatcaggtctggtgcagaccatagcatacatcaaactcccaactgcagaagcataaggtacttttgccatgtgatcacgctccatagttgtcttgggtgattgctcatttgacaatttgagatggctagccaaaggcgtgctcctggacttagtatcatcaattttgaacctggacaacaccttctgaatgtattgctcctgagataaatttagaattccagcagacctgtcccggaaaagcctcatccctaagaatatgctttgctgcatccaaatcttttatctcaaactctaatgacagtctccccttttgagttcgatcgaccatgtttaccatgcccacctttcaccttactcctgcctctaccctcgacactaagtggatctacgtgaactgctaattgcgaaaacaatagtatcccacgactcaggtagagaggataacaaaattagatttcttatctcatcatcaaaattaatcttgagagagcttaattgactgattatcatattaaattcgttaatatgatccgagatagattcacttttagacatctccaaattaatcaaacaatgcatcagatatattttgttaatggttgacggtttctcatatatgttcgctagacctgtcaacaatcgtacagttgttgtctccttaacgatattgaaggcaccgtttctcgccaacgtcaaatggatcaaccctagtgcctttcaatccttcaaatcccaaacctcctgtttcatagtttctggcttctctcctgacaagggttcatggaggttattctgatacaggtaatcttcaatctgcatctttcagaaaccaaaatctattccatcgaatttctcaattcgaatcttcgggctttccattgtcttttcagattgttgtgatacaaaccaaaagctctgataccagttgttaagaatcgaacagtcacacaatgtaaaaataagcacgaaaagaaattaacacataaatttaacgtggttcaccaatttagttacttccacggctactaagtttttttatttctctgagtttttgaaaccgaatacgctaggagaatatattattaggctccatatctcaacaataacttcatttttaacgaaattcatcatttttagtcatcatttcctataccttctttttcaggttaagctctctggttcaattagcagtcaatacttgactgctttgctcatggcagctcctttggctcttggagatgtagaaattgagattattgataaattgatatccattccttatgttgatatgactttgaagttaatggaacgattcggagtcactgcagaacacagtggtagctgggatcgtttcttcattcgtgggggtcaaaagtacaagtatgtttttttcaatatagatctgcaagctttttactgttgtttgactgaaattaacaacctgaaaaatcaattagatctcctggaaaagcttttgttgaaggtgatgcttcgagtgccagttatttcctggctggtgcagcaatcactggtggaaccatcactgttgaaggttgtggaacaagtagtttgcaggtatttcactcattaatatactgtcttactgcttcctataatcttaaaatggcttgtggaagtgcttattgtatccattgctttttgatgtttgaaaatttcgtcaaaattttctcaacatacacatgtatatatgatgtcttctccatggctccctactgtctaatatttcctaggaggaagtttgcaacataattgtatgcatttacacttaccaaaccaacttgcttagtttccttatctttatgaaaatcaagggggatgtaaggtttgctgaagtgcttgacaaaatgggagcaaaagtcacctggacagagaacagtgttactgtgactggaccaccacgcaattctcctattaaaaagcacttgcgagctattgatgtcaacatgaacaaaatgccagatgttgctatgacattggccgtggttgcactttttgctgatggccctactgccataagagacggtataatttgtccttcaaactttgtgaataccagtcatattctttcaataaaatgatcatgcagtacataaacaatgaactggtgattattatttggtggatcatagaacagctttggcttctattcatggaagatggcaatgaatgtaacccaattctaaatcattgaaatcccatttcttgggtccggacttcttgattaatacaaaatggtagtggctaaaagtaggaagcttcagctcatggaggatgaagtttctgaaggaaaaaatggtggctgtttcttttctttatctaataacagctagaacttccatatttgaccttcggtggatatttaagatctaaaacattgttatcttttcatctgaatcaaatcatcagcaggtttcacatatgtttccttgtaacttgtatgccaatgccaatctcttgatttctcttatttccagtggccagttggagagttaaagaaacagaacggatgattgcaatttgcacagagctcaggaaggtcagtttctggcagttcttaaactcatctgaatttcatgcaaacaaaaggtggtttatatttttttcttatataaatgacatttgggctgcaaatttcagctcggagcaaatgttgaagagggaccagactactgtttgatcactccacctgagaaactgaacatcacagagattgacacttatgatgatcacagaatggccatggcgttctcccttgctgcctgcggggatgttccagtcacaatcaaggaccctggttgcacaagaaaaacttttccagactactttgaggtccttgagaggttcactaagcatgattataaggatcacgatggcgactacaaggaccacgatattgactacaaagacgatgacgataaatgagccacacttcaacagtcacaattaataatagagagataggtttaccagcagtgtcactgcaactatgatgaagcaacctatatgtaataattttccccacctgattgcccattgtaatctttgataatttatgcgaggtgatgtttgttagtgtcccagccaaattgtccccaggtgactcgaagttacgtataggttatccagtgaatgcttggggtttagcgaatacttcattggtttctggaacgagacaatcgaaattggaaacacagtcgaccactcggtcataaaatgatttgatagtgaaagcattatttaaagtgatttgatagtggatcataaaatgttaacttagctaaccgttgataattattaaaacaattagtagttgaaagtacatagaatcatatagttccgggcctagttgcatcggccaatatatataaaatataaataagggaagtaattattcagaggtgcagacgttgtgcattatatttgtaacagcttgtgagatgggttggaggttggaggtggctccgggagatttatttctttgctttttcttcaaaatgtaaactaatgaacaagtgtttggggaggaaaaggagtacaagggactgtcaggactcagctatctacccggatcctctagagtcgacctgcaggcatgcaagcttggcgcgccattaaattaaactatgagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtactgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcgcgagctgttggctggctggtggcaggatatattgtggtgtaaacacgagctgttggctggctggtggcaggatatattgtggtgtaaacacgagctgttggctggctggtggcaggatatattgtggtgtaaacaaattgacgcttagacaacttaataacacattgcggacgtttttaatgtactgaatttacgccgaat

SEQ ID NO: 9 - DNA, 16503 bp, artificial
  cloning vector including a repair template for use with either the NHEJ or HR repair pathways
gacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgggagagcgcccatatgcgcactcctcgcatgcggctcgagtggagctgggcccacgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagcttcaaagctcatcatgtttgtatagttcatccatgccatgtgtaatcccagcagctgttacaaactcaagaaggaccatgtggtctctcttttcgttgggatctttcgaaagggcagattgatgggacaggtaatggttgtctggtaaaaggacagggccatcgccaattggagtattttgttgataatggtctgctagttgaacgcttccatcttcaacgttgtgtctaattttgaagttagctttgattccattcttttgtttgtctgccgtgatgtatacgttgtgggagttaaagttgtattccaacttgtggccgaggatgtttccgtcctccttgaaatcgattcccttaagctcgatcctgttgacgagggtgtctccctcaaacttgacttcagcacgtgtcttgtaagtcccgtcgtccttgaaagagatggtcctctcctgcacgtatccctcaggcatggcgctcttgaagaagtcgtgttgcttcatatgatctgggtatcttgaaaagcaagcaacaccatatccgaaagtagtgacaagtgttggccatggaacaggtagttttccagtagtgcaaataaatttaagggtaagttttccatttgttgcatcaccttcaccctctcctctgacagaaaatttgtgcccattaacatcaccatctaattcaacaagaattgggacaactccagtgaaaagttcttctcctttactgaattcggccgaggataatgattggagaagtgaaaagatgagaaagagaaaaagattagtcttcattctagagtcccccgtgttctctccaaatgaaatgaacttccttatatagaggaagggtcttgcgaaaggatagtgggattgtgcgtcatccccttacgtcagtggagatatcacatcaatccacttgcttttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagacctgctgcgtaagcctctctaaccatctgtgggttagcattctttctgaaattgaaaaggcaaatctggggaccgtttaaactatcaggtgacgtacccaaagctctggaagttctcggcgcgccttagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcggtaccgatcggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgactcgagatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataaggagctccggtgacggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtcagaactcataagttgggtgttttagagctagaaatagcaagttaaaataaggctagtccgttatcaacttgaaaaagtggcaccgagtcggtgctcccggcgccatggatgttgttgttaccagaaagtaaataaatgttcaatctctgatgttctcaagtaagtgagttttattgggaataatattaacttatgttcttcttgcatttgatttctttgccgctctcttcttctatcttaaatctgtgtatactatttcactattgggctttttattagtctataatgggactcaaaataaggctttggcccacatcaaaaagataagtcacaaatcaaaactaaattcagagtcttttctcccacatcggtcactgtactcattttgtgtttgtttatatattacacgaaccgatctttggtacgatatactatacatcaaatgggttttagagctagaaatagcaagttaaaataaggctagtccgttatcaacttgaaaaagtggcaccgagtcggtgctttttttcgcgcgtagtcctcggtaatatcgcagaacaaaagtacctgatggacccacggaattgtgtcagtgaatgctaatgagtgttattgttatctggttgggacggagaaagattgacctatttcaaaagttgtggttgttggaactcgtattctcagccacctatcctatcctcattgtcctctttccacttgccagcattcccaccgccaccaccagtggcactgggatgcggggaaatggatgttcttaattgcttttatttttatttttatttttattttttgaaaaaatcttatgataaaaatatattaggcttgctttttgagttaatttttaataatctacattaatatcttgtacctttttaaataataatttcggcaaaaccagatataataaaatctctcatagaaaatctatttttgtgatacagactcaccaacgaattttttaggcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggagcccgaaagtcagaactcataagttgggtgggacatcagtcgacggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatctgtcgatcgaccatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtgctttgactgctgctgttacggcagcaggaggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgccaacccaacttatgagttctgaggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgatcgagtgtacttcaagtcacaccggcgagtgtttgatcgccggcggtaccgagtgtacttcaagtcagtgggaaatcaataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggagaagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcatagtttagtgccagccag

SEQ ID NO: 10 - DNA, 16517 bp, artificial
  cloning vector including a repair template for use with the HR repair pathway
gacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgggagagcgcccatatgcgcactcctcgcatgcggctcgagtggagctgggcccacgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagcttcaaagctcatcatgtttgtatagttcatccatgccatgtgtaatcccagcagctgttacaaactcaagaaggaccatgtggtctctcttttcgttgggatctttcgaaagggcagattgatgggacaggtaatggttgtctggtaaaaggacagggccatcgccaattggagtattttgttgataatggtctgctagttgaacgcttccatcttcaacgttgtgtctaattttgaagttagctttgattccattcttttgtttgtctgccgtgatgtatacgttgtgggagttaaagttgtattccaacttgtggccgaggatgtttccgtcctccttgaaatcgattcccttaagctcgatcctgttgacgagggtgtctccctcaaacttgacttcagcacgtgtcttgtaagtcccgtcgtccttgaaagagatggtcctctcctgcacgtatccctcaggcatggcgctcttgaagaagtcgtgttgcttcatatgatctgggtatcttgaaaagcaagcaacaccatatccgaaagtagtgacaagtgttggccatggaacaggtagttttccagtagtgcaaataaatttaagggtaagttttccatttgttgcatcaccttcaccctctcctctgacagaaaatttgtgcccattaacatcaccatctaattcaacaagaattgggacaactccagtgaaaagttcttctcctttactgaattcggccgaggataatgattggagaagtgaaaagatgagaaagagaaaaagattagtcttcattctagagtcccccgtgttctctccaaatgaaatgaacttccttatatagaggaagggtcttgcgaaaggatagtgggattgtgcgtcatccccttacgtcagtggagatatcacatcaatccacttgcttttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagacctgctgcgtaagcctctctaaccatctgtgggttagcattctttctgaaattgaaaaggcaaatctggggaccgtttaaactatcaggtgacgtacccaaagctctggaagttctcggcgcgccttagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcggtaccgatcggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgactcgagatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataaggagctccggtgacggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtcagaactcataagttgggtgttttagagctagaaatagcaagttaaaataaggctagtccgttatcaacttgaaaaagtggcaccgagtcggtgctttttttcccggcgccatggatgttgttgttaccagaaagtaaataaatgttcaatctctgatgttctcaagtaagtgagttttattgggaataatattaacttatgttcttcttgcatttgatttctttgccgctctcttcttctatcttaaatctgtgtatactatttcactattgggctttttattagtctataatgggactcaaaataaggctttggcccacatcaaaaagataagtcacaaatcaaaactaaattcagagtcttttctcccacatcggtcactgtactcattttgtgtttgtttatatattacacgaaccgatctttggtacgatatactatacatcaaatgggttttagagctagaaatagcaagttaaaataaggctagtccgttatcaacttgaaaaagtggcaccgagtcggtgctttttttcgcgcgtagtcctcggtaatatcgcagaacaaaagtacctgatggacccacggaattgtgtccgcagaacaaaagtacctgatggacccacggaattgtgtcagtgaatgctaatgagtgttattgttatctggttgggacggagaaagattgacctatttcaaaagttgtggttgttggaactcgtattctcagccacctatcctatcctcattgtcctctttccacttgccagcattcccaccgccaccaccagtggcactgggatgcggggaaatggatgttcttaattgcttttatttttatttttatttttattttttgaaaaaatcttatgataaaaatatattaggcttgctttttgagttaatttttaataatctacattaatatcttgtacctttttaaataataatttcggcaaaaccagatataataaaatctctcatagaaaatctatttttgtgatacagactcaccaacgaattttttaggcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggagcccgaaagtcgacggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtgctttgactgctgctgttacggcagcaggaggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgttcagaactcataagttgttggggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgatcgagtgtacttcaagtcacaccggcgagtgtttgatcgccggcggtaccgagtgtacttcaagtcagtgggaaatcaataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggagaagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcatagtttagtgccagccag

SEQ ID NO: 11 - DNA, 16900 bp, artificial
  T-DNA vector including a repair template for use with either the NHEJ or HR repair pathways
aattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtttccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagaggccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagccgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttttcgtacgaccctcggtaccgatcggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgactcgagatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataaggagctccggtgacggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtcagaactcataagttgggtgttttagagctagaaatagcaagttaaaataaggctagtccgttatcaacttgaaaaagtggcaccgagtcggtgctttttttttcccggcgccatggatgttgttgttaccagaaagtaaataaatgttcaatctctgatgtttctcaagtaagtgagttttattgggaataatattaacttatgttcttcttgcatttgatttctttgccgctctcttcttctatcttaaatctgtgtatactatttcactattgggctttttattagtctataatgggactcaaaataaggctttggcccacatcaaaaagataagtcacaaatcaaaactaaattcagagtcttttctcccacatcggtcactgtactcattttgtgtttgtttatatattacacgaaccgatctttggtacgatatactatacatcaaatgggttttagagctagaaatagcaagttaaaataaggctagtccgttatcaacttgaaaaagtggcaccgagtcggtgctttttttcgcgcgtagtcctcggtaatatcgcagaacaaaagtacctgatggacccacggaattgtgtcagtgaatgctaatgagtgttattgttatctggttgggacggagaaagattgacctatttcaaaagttgtggttgttggaactcgtattctcagccacctatcctatcctcattgtcctctttccacttgccagcattcccaccgccaccaccagtggcactgggatgcggggaaatggatgttcttaattgcttttatttttatttttatttttattttttgaaaaaatcttatgataaaaatatattaggcttgctttttgagttaatttttaataatctacattaatatcttgtacctttttaaataataatttcggcaaaaccagatataataaaatctctcatagaaaatctatttttgtgatacagactcaccaacgaattttttaggcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggagcccgaaagtcagaactcataagttgggtgggacatcagtcgacggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtgctttgactgctgctgttacggcagcaggaggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgccaacccaacttatgagttctgaggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgatcgagtgtacttcaagtcacaccggcgagtgtttgatcgccggcggtaccgagtgtacttcaagtcactc

SEQ ID NO: 12 - DNA, 16906 bp, artificial
  T-DNA vector including a repair template for use with the HR repair pathway
aattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtttccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagaggccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagccgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttttcgtacgaccctcggtaccgatcggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgactcgagatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataaggagctccggtgacggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtcagaactcataagttgggtgttttagagctagaaatagcaagttaaaataaggctagtccgttatcaacttgaaaaagtggcaccgagtcggtgctttttttcccggcgccatggatgttgttgttaccagaaagtaaataaatgttcaatctctgatgttctcaagtaagtgagttttattgggaataatattaacttatgttcttcttgcatttgatttctttgccgctctcttcttctatcttaaatctgtgtatactatttcactattgggctttttattagtctataatgggactcaaaataaggctttggcccacatcaaaaagataagtcacaaatcaaaactaaattcagagtcttttctcccacatcggtcactgtactcattttgtgtttgtttatatattacacgaaccgatctttggtacgatatactatacatcaaatgggttttagagctagaaatagcaagttaaaataaggctagtccgttatcaacttgaaaaagtggcaccgagtcggtgctttttttcgcgcgtagtcctcggtaatatcgcagaacaaaagtacctgatggacccacggaattgtgtccgcagaacaaaagtacctgatggacccacggaattgtgtcagtgaatgctaatgagtgttattgttatctggttgggacggagaaagattgacctatttcaaaagttgtggttgttggaactcgtattctcagccacctatcctatcctcattgtcctctttccacttgccagcattcccaccgccaccaccagtggcactgggatgcggggaaatggatgttcttaattgcttttatttttatttttatttttattttttgaaaaaatcttatgataaaaatatattaggcttgctttttgagttaatttttaataatctacattaatatcttgtacctttttaaataataatttcggcaaaaccagatataataaaatctctcatagaaaatctatttttgtgatacagactcaccaacgaattttttaggcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggagcccgaaagtcgacggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtgctttgactgctgctgttacggcagcaggaggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgttcagaactcataagttgttggggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgatcgagtgtacttcaagtcacaccggcgagtgtttgatcgccggcggtaccgagtgtacttcaagtcactc

SEQ ID NO: 13 - DNA, 16895 bp, artificial
  T-DNA vector including a negative control nuclease
aattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtttccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagaggccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagccgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttttcgtacgaccctcggtaccgatcggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgactcgagatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataaggagctccggtgacggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgctgccagcctgatctttgagttttagagctagaaatagcaagttaaaataaggctagtccgttatcaacttgaaaaagtggcaccgagtcggtgctttttttcccggcgccatggatgttgttgttaccagaaagtaaataaatgttcaatctctgatgttctcaagtaagtgagttttattgggaataatattaacttatgttcttcttgcatttgatttctttgccgctctcttcttctatcttaaatctgtgtatactatttcactattgggctttttattagtctataatgggactcaaaataaggctttggcccacatcaaaaagataagtcacaaatcaaaactaaattcagagtcttttctcccacatcggtcactgtactcattttgtgtttgtttatatattacacgaaccgatctttggtacgtaaagttttctggttaattgttttagagctagaaatagcaagttaaaataaggctagtccgttatcaacttgaaaaagtggcaccgagtcggtgctttttttcgcgcgtagtcctcggtaatatcgcagaacaaaagtacctgatggacccacggaattgtgtcagtgaatgctaatgagtgttattgttatctggttgggacggagaaagattgacctatttcaaaagttgtggttgttggaactcgtattctcagccacctatcctatcctcattgtcctctttccacttgccagcattcccaccgccaccaccagtggcactgggatgcggggaaatggatgttcttaattgcttttatttttatttttatttttattttttgaaaaaatcttatgataaaaatatattaggcttgctttttgagttaatttttaataatctacattaatatcttgtacctttttaaataataatttcggcaaaaccagatataataaaatctctcatagaaaatctatttttgtgatacagactcaccaacgaattttttaggcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggagcccgaaagtcagaactcataagttgggtgggacatcagtcgacggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcaataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtgctttgactgctgctgttacggcagcaggaggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgccaacccaacttatgagttctgaggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgatcgagtgtacttcaagtcacaccggcgagtgtttgatcgccggcggtaccgagtgtacttcaagtcactc

SEQ ID NO: 14 - DNA, 18864 bp, artificial
  T-DNA vector including a geminivirus replicon
gtgggaaatcaataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggagaagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcatagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacggctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggcctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggaaacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtagagctcaaagtttaacgcgttagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcggtaccgatcggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgactcgagatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataaggagctccggtgacggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtcagaactcataagttgggtgttttagagctagaaatagcaagttaaaataaggctagtccgttatcaacttgaaaaagtggcaccgagtcggtgctttttttcccggcgccatggatgttgttgttaccagaaagtaaataaatgttcaatctctgatgttctcaagtaagtgagttttattgggaataatattaacttatgttcttcttgcatttgatttctttgccgctctcttcttctatcttaaatctgtgtatactatttcactattgggctttttattagtctataatgggactcaaaataaggctttggcccacatcaaaaagataagtcacaaatcaaaactaaattcagagtcttttctcccacatcggtcactgtactcattttgtgtttgtttatatattacacgaaccgatctttggtacgatatactatacatcaaatgggttttagagctagaaatagcaagttaaaataaggctagtccgttatcaacttgaaaaagtggcaccgagtcggtgctttttttcgcgcgtagtcctcggtaatatcgcagaacaaaagtacctgatggacccacggaattgtgtcagtgaatgctaatgagtgttattgttatctggttgggacggagaaagattgacctatttcaaaagttgtggttgttggaactcgtattctcagccacctatcctatcctcattgtcctctttccacttgccagcattcccaccgccaccaccagtggcactgggatgcggggaaatggatgttcttaattgcttttatttttatttttatttttattttttgaaaaaatcttatgataaaaatatattaggcttgctttttgagttaatttttaataatctacattaatatcttgtacctttttaaataataatttcggcaaaaccagatataataaaatctctcatagaaaatctatttttgtgatacagactcaccaacgaattttttaggcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggagcccgaaagtcagaactcataagttgggtgggacatcagtcgacggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtgctttgactgctgctgttacggcagcaggaggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgccaacccaacttatgagttctgaggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgatcgagtgtacttcaagtcacaccggcgagtgtttgatcgccggcggtaccgagtgtacttcaagtca

SEQ ID NO: 15 - DNA, 17210 bp, artificial
  T-DNA vector including a repair template designed to incorporate the cassava cold, circadian rhythm, RNA binding 1 annotated promoter into the edited target site to drive T102I/P106A EPSPS expression
aattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtttccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagaggccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagccgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttttcgtacgaccctcggtaccgatcggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgactcgagatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataaggagctccggtgacggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtcagaactcataagttgggtgttttagagctagaaatagcaagttaaaataaggctagtccgttatcaacttgaaaaagtggcaccgagtcggtgctttttttttcccggcgccatggatgttgttgttaccagaaagtaaataaatgttcaatctctgatgtttctcaagtaagtgagttttattgggaataatattaacttatgttcttcttgcatttgatttctttgccgctctcttcttctatcttaaatctgtgtatactatttcactattgggctttttattagtctataatgggactcaaaataaggctttggcccacatcaaaaagataagtcacaaatcaaaactaaattcagagtcttttctcccacatcggtcactgtactcattttgtgtttgtttatatattacacgaaccgatctttggtacgatatactatacatcaaatgggttttagagctagaaatagcaagttaaaataaggctagtccgttatcaacttgaaaaagtggcaccgagtcggtgctttttttcgcgcgtagtcctcggtaatatcgcagaacaaaagtacctgatggacccacggaattgtgtcagtgaatgctaatgagtgttattgttatctggttgggacggagaaagattgacctatttcaaaagttgtggttgttggaactcgtattctcagccacctatcctatcctcattgtcctctttccacttgccagcattcccaccgccaccaccagtggcactgggatgcggggaaatggatgttcttaattgcttttatttttatttttatttttattttttgaaaaaatcttatgataaaaatatattaggcttgctttttgagttaatttttaataatctacattaatatcttgtacctttttaaataataatttcggcaaaaccagatataataaaatctctcatagaaaatctatttttgtgatacagactcaccaacgaattttttaggcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggagcccgaaagtcagaactcataagttgggtgggacatcagcactacaattacgtgttgtgtagcataaatggggcatatttgattatatataatttcataaatctcaaatacaataaaactattcacagtactgataaaaataagaataattcatttattttttgaataacttttaattatgagtatttgattttgagttctatttcatttatattgctcagtttttatttattttaataaagttacttaatttttatttttattttaaataaaattattttaacttgttacgacgaaattttagattaataaccactatattcattctcctttttagtataatattttatttttaagtaattgatagttatttattaataatatcaattgatttttaatatatttattattagattttataatcaattaaagaaattaaattaatgtcttcttttcttctattttttctaaaaaataattttttatatatattaactaataaaaaaaagacaagattttctgaaaggcattctaaataattgaattttattaatataattttttttaattagattctatttgtttatggaaaataatttttttaataaaataatttatttttaattatttaatttaaaaataaatatattaatataaaattttaataaaaattttaaagtctaaaaaataatttaattttttaattaaaaaaatatttttatttaaatataaaaaattataaaaaaaaaatattttcattttcctaaaagaaatgaagatttaatttagacaaattggagaaaaaaatttagacaattaaggaattccataaactcagagggacaaaattgtacttgcacctatcaagatattttcgaagactataaaatatcagcaactttcatccctatctatccacccacctggccactatcagcgtgattgtggaccccacttgactcgggtcaccgcttttgttaaccatggtttaccattccacaaatactgaccacttgccttcggattttattcgaatatactcgcggcacctttcgctattcccctaatccctgttctgaataatcacatcttttgaaccaaaaaccaggttaagtttatcgctgtaaccaaacatccttcacattcaattaaccatagttaatttcctccctctataaaaccatggtttagccttgctttgtttctcacaatctcctctcttggcttgtgttctctcttatttttcagagatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtgctttgactgctgctgttacggcagcaggaggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgccaacccaacttatgagttctgaggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgatcgagtgtacttcaagtcacaccggcgagtgtttgatcgccggcggtaccgagtgtacttcaagtcactc

SEQ ID NO: 16 - DNA, 19174 bp, artificial
  geminivirus replicon T-DNA vector including a repair template designed to incorporate the cassava cold, circadian rhythm, RNA binding 1 annotated promoter into the edited target site to drive T102I/P106A EPSPS expression
gtgggaaatcaataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggagaagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcatagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacggctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggcctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggaaacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtagagctcaaagtttaacgcgttagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcggtaccgatcggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgactcgagatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataaggagctccggtgacggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtcagaactcataagttgggtgttttagagctagaaatagcaagttaaaataaggctagtccgttatcaacttgaaaaagtggcaccgagtcggtgctttttttcccggcgccatggatgttgttgttaccagaaagtaaataaatgttcaatctctgatgttctcaagtaagtgagttttattgggaataatattaacttatgttcttcttgcatttgatttctttgccgctctcttcttctatcttaaatctgtgtatactatttcactattgggctttttattagtctataatgggactcaaaataaggctttggcccacatcaaaaagataagtcacaaatcaaaactaaattcagagtcttttctcccacatcggtcactgtactcattttgtgtttgtttatatattacacgaaccgatctttggtacgatatactatacatcaaatgggttttagagctagaaatagcaagttaaaataaggctagtccgttatcaacttgaaaaagtggcaccgagtcggtgctttttttcgcgcgtagtcctcggtaatatcgcagaacaaaagtacctgatggacccacggaattgtgtcagtgaatgctaatgagtgttattgttatctggttgggacggagaaagattgacctatttcaaaagttgtggttgttggaactcgtattctcagccacctatcctatcctcattgtcctctttccacttgccagcattcccaccgccaccaccagtggcactgggatgcggggaaatggatgttcttaattgcttttatttttatttttatttttattttttgaaaaaatcttatgataaaaatatattaggcttgctttttgagttaatttttaataatctacattaatatcttgtacctttttaaataataatttcggcaaaaccagatataataaaatctctcatagaaaatctatttttgtgatacagactcaccaacgaattttttaggcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggagcccgaaagtcagaactcataagttgggtgggacatcagcactacaattacgtgttgtgtagcataaatggggcatatttgattatatataatttcataaatctcaaatacaataaaactattcacagtactgataaaaataagaataattcatttattttttgaataacttttaattatgagtatttgattttgagttctatttcatttatattgctcagtttttatttattttaataaagttacttaatttttatttttattttaaataaaattattttaacttgttacgacgaaattttagattaataaccactatattcattctcctttttagtataatattttatttttaagtaattgatagttatttattaataatatcaattgatttttaatatatttattattagattttataatcaattaaagaaattaaattaatgtcttcttttcttctattttttctaaaaaataattttttatatatattaactaataaaaaaaagacaagattttctgaaaggcattctaaataattgaattttattaatataattttttttaattagattctatttgtttatggaaaataatttttttaataaaataatttatttttaattatttaatttaaaaataaatatattaatataaaattttaataaaaattttaaagtctaaaaaataatttaattttttaattaaaaaaatatttttatttaaatataaaaaattataaaaaaaaaatattttcattttcctaaaagaaatgaagatttaatttagacaaattggagaaaaaaatttagacaattaaggaattccataaactcagagggacaaaattgtacttgcacctatcaagatattttcgaagactataaaatatcagcaactttcatccctatctatccacccacctggccactatcagcgtgattgtggaccccacttgactcgggtcaccgcttttgttaaccatggtttaccattccacaaatactgaccacttgccttcggattttattcgaatatactcgcggcacctttcgctattcccctaatccctgttctgaataatcacatcttttgaaccaaaaaccaggttaagtttatcgctgtaaccaaacatccttcacattcaattaaccatagttaatttcctccctctataaaaccatggtttagccttgctttgtttctcacaatctcctctcttggcttgtgttctctcttatttttcagagatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtgctttgactgctgctgttacggcagcaggaggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgccaacccaacttatgagttctgaggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgatcgagtgtacttcaagtcacaccggcgagtgtttgatcgccggcggtaccgagtgtacttcaagtca

SEQ ID NO: 17 - DNA, 17301 bp, artificial
  T-DNA vector including a repair template designed to incorporate the cassava dehydrin family annotated promoter into the edited target site to drive T102I/P106A EPSPS expression
aattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtttccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagaggccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagccgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttttcgtacgaccctcggtaccgatcggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgactcgagatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataaggagctccggtgacggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtcagaactcataagttgggtgttttagagctagaaatagcaagttaaaataaggctagtccgttatcaacttgaaaaagtggcaccgagtcggtgctttttttttcccggcgccatggatgttgttgttaccagaaagtaaataaatgttcaatctctgatgtttctcaagtaagtgagttttattgggaataatattaacttatgttcttcttgcatttgatttctttgccgctctcttcttctatcttaaatctgtgtatactatttcactattgggctttttattagtctataatgggactcaaaataaggctttggcccacatcaaaaagataagtcacaaatcaaaactaaattcagagtcttttctcccacatcggtcactgtactcattttgtgtttgtttatatattacacgaaccgatctttggtacgatatactatacatcaaatgggttttagagctagaaatagcaagttaaaataaggctagtccgttatcaacttgaaaaagtggcaccgagtcggtgctttttttcgcgcgtagtcctcggtaatatcgcagaacaaaagtacctgatggacccacggaattgtgtcagtgaatgctaatgagtgttattgttatctggttgggacggagaaagattgacctatttcaaaagttgtggttgttggaactcgtattctcagccacctatcctatcctcattgtcctctttccacttgccagcattcccaccgccaccaccagtggcactgggatgcggggaaatggatgttcttaattgcttttatttttatttttatttttattttttgaaaaaatcttatgataaaaatatattaggcttgctttttgagttaatttttaataatctacattaatatcttgtacctttttaaataataatttcggcaaaaccagatataataaaatctctcatagaaaatctatttttgtgatacagactcaccaacgaattttttaggcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggagcccgaaagtcagaactcataagttgggtgggacatcagttcgctatggtatcggcaattatggcgacagcaacggttactctacccatcagaatcaccatatccaaatttagatattaaatatcaatttcgaactcaattaaaatttaccccatcagaatcaccatatccaaacttagatattaaatatcaatttcgaactcaattaaaatttactttaaataattaaattaattaatattattatttataagacattcacactcatttacttttatgaattttattaatttatcaaaaaactttaaaaataaatattgaaataagggataaatgaaatttttacaaaattatatataattttaatttttaaataaattatttaaattaaaaaattaatccttttcttttaattaaaaattaattaaattatatttttataaaattctcaatttcaaaaaatatgaaatgatttgacagttgctccatataaaatcaaagttatttttatcactattaaccaaattctatccttcgttgttagaagaacagagaggacaatgacctttctggcaccacaaagccactctccatttcgccacaacatttggcatcacaatatcctcttatttttgctttcttttaaagagaatatatacatacaatgaataatcaagacctatataaaattatacaagtttaataaatatcagcctctaatttttaggtttattttaaacagctgtgggctaattctcaaatagtaaatctaatgttagtggttttgctgtaaccatggagaaattaattaccctaatctaataaccaaatacattggatctgaccatcatctttatttaatgaattttatgcctatatttgtgcctgtaaatggggacgtaaagaactttctatgtcccttcaaatctgacatatccaaatgcattttttaattaaaaaaattactatttaatctcaaatttttattaatttctaaatattttttaaattttaaaaaatttaattaatcgggtcgttaacaaaagttaaccgttctaatcaaatgagactttaatatttaaaattaataaaataattaattagtatatttttaaaattttcgtgtagaatccgtgtaaaagggggggcatttatgtgatttcaattcatcggataactgtatacacacatattttttataaatgggaaaatttctttattctcctcctctttatactgattggcctataaataaacaagttctccacccgctttttctcatctcatctcatcagatcagagtcacagaagcaaaagctttgatttcagaagaaagagaaaatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtgctttgactgctgctgttacggcagcaggaggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgccaacccaacttatgagttctgaggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgatcgagtgtacttcaagtcacaccggcgagtgtttgatcgccggcggtaccgagtgtacttcaagtcactc

SEQ ID NO: 18 - DNA, 19265 bp, artificial
  geminivirus replicon T-DNA vector including a repair template designed to incorporate the cassava dehydrin family annotated promoter into the edited target site to drive T102I/P106A EPSPS expression
gtgggaaatcaataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggagaagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcatagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacggctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggcctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggaaacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtagagctcaaagtttaacgcgttagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcggtaccgatcggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgactcgagatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataaggagctccggtgacggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtcagaactcataagttgggtgttttagagctagaaatagcaagttaaaataaggctagtccgttatcaacttgaaaaagtggcaccgagtcggtgctttttttcccggcgccatggatgttgttgttaccagaaagtaaataaatgttcaatctctgatgttctcaagtaagtgagttttattgggaataatattaacttatgttcttcttgcatttgatttctttgccgctctcttcttctatcttaaatctgtgtatactatttcactattgggctttttattagtctataatgggactcaaaataaggctttggcccacatcaaaaagataagtcacaaatcaaaactaaattcagagtcttttctcccacatcggtcactgtactcattttgtgtttgtttatatattacacgaaccgatctttggtacgatatactatacatcaaatgggttttagagctagaaatagcaagttaaaataaggctagtccgttatcaacttgaaaaagtggcaccgagtcggtgctttttttcgcgcgtagtcctcggtaatatcgcagaacaaaagtacctgatggacccacggaattgtgtcagtgaatgctaatgagtgttattgttatctggttgggacggagaaagattgacctatttcaaaagttgtggttgttggaactcgtattctcagccacctatcctatcctcattgtcctctttccacttgccagcattcccaccgccaccaccagtggcactgggatgcggggaaatggatgttcttaattgcttttatttttatttttatttttattttttgaaaaaatcttatgataaaaatatattaggcttgctttttgagttaatttttaataatctacattaatatcttgtacctttttaaataataatttcggcaaaaccagatataataaaatctctcatagaaaatctatttttgtgatacagactcaccaacgaattttttaggcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggagcccgaaagtcagaactcataagttgggtgggacatcagttcgctatggtatcggcaattatggcgacagcaacggttactctacccatcagaatcaccatatccaaatttagatattaaatatcaatttcgaactcaattaaaatttaccccatcagaatcaccatatccaaacttagatattaaatatcaatttcgaactcaattaaaatttactttaaataattaaattaattaatattattatttataagacattcacactcatttacttttatgaattttattaatttatcaaaaaactttaaaaataaatattgaaataagggataaatgaaatttttacaaaattatatataattttaatttttaaataaattatttaaattaaaaaattaatccttttcttttaattaaaaattaattaaattatatttttataaaattctcaatttcaaaaaatatgaaatgatttgacagttgctccatataaaatcaaagttatttttatcactattaaccaaattctatccttcgttgttagaagaacagagaggacaatgacctttctggcaccacaaagccactctccatttcgccacaacatttggcatcacaatatcctcttatttttgctttcttttaaagagaatatatacatacaatgaataatcaagacctatataaaattatacaagtttaataaatatcagcctctaatttttaggtttattttaaacagctgtgggctaattctcaaatagtaaatctaatgttagtggttttgctgtaaccatggagaaattaattaccctaatctaataaccaaatacattggatctgaccatcatctttatttaatgaattttatgcctatatttgtgcctgtaaatggggacgtaaagaactttctatgtcccttcaaatctgacatatccaaatgcattttttaattaaaaaaattactatttaatctcaaatttttattaatttctaaatattttttaaattttaaaaaatttaattaatcgggtcgttaacaaaagttaaccgttctaatcaaatgagactttaatatttaaaattaataaaataattaattagtatatttttaaaattttcgtgtagaatccgtgtaaaagggggggcatttatgtgatttcaattcatcggataactgtatacacacatattttttataaatgggaaaatttctttattctcctcctctttatactgattggcctataaataaacaagttctccacccgctttttctcatctcatctcatcagatcagagtcacagaagcaaaagctttgatttcagaagaaagagaaaatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtgctttgactgctgctgttacggcagcaggaggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgccaacccaacttatgagttctgaggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgatcgagtgtacttcaagtcacaccggcgagtgtttgatcgccggcggtaccgagtgtacttcaagtca

SEQ ID NO: 19 - DNA, 20395 bp, artificial
  T-DNA vector including a repair template designed to incorporate the cassava rotamase CYP1 annotated promoter into the edited target site to drive T102I/P106A EPSPS expression
  misc_feature (14362)..(14388): n is a, c, g, or t
aattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtttccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagaggccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagccgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttttcgtacgaccctcggtaccgatcggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgactcgagatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataaggagctccggtgacggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtcagaactcataagttgggtgttttagagctagaaatagcaagttaaaataaggctagtccgttatcaacttgaaaaagtggcaccgagtcggtgctttttttttcccggcgccatggatgttgttgttaccagaaagtaaataaatgttcaatctctgatgtttctcaagtaagtgagttttattgggaataatattaacttatgttcttcttgcatttgatttctttgccgctctcttcttctatcttaaatctgtgtatactatttcactattgggctttttattagtctataatgggactcaaaataaggctttggcccacatcaaaaagataagtcacaaatcaaaactaaattcagagtcttttctcccacatcggtcactgtactcattttgtgtttgtttatatattacacgaaccgatctttggtacgatatactatacatcaaatgggttttagagctagaaatagcaagttaaaataaggctagtccgttatcaacttgaaaaagtggcaccgagtcggtgctttttttcgcgcgtagtcctcggtaatatcgcagaacaaaagtacctgatggacccacggaattgtgtcagtgaatgctaatgagtgttattgttatctggttgggacggagaaagattgacctatttcaaaagttgtggttgttggaactcgtattctcagccacctatcctatcctcattgtcctctttccacttgccagcattcccaccgccaccaccagtggcactgggatgcggggaaatggatgttcttaattgcttttatttttatttttatttttattttttgaaaaaatcttatgataaaaatatattaggcttgctttttgagttaatttttaataatctacattaatatcttgtacctttttaaataataatttcggcaaaaccagatataataaaatctctcatagaaaatctatttttgtgatacagactcaccaacgaattttttaggcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggagcccgaaagtcagaactcataagttgggtgggacatcagtaatagctttgtgtcctggtctaaaggcattgatgggtccctgcaaaagaggatttcattgcgtagtttttgaaagataggcaaagtggtagagatttatatggaataatgactattttttaatccatatacttttttttttatatttgaatccattatatattttgtctttttataatttaatagttgaattgttcaaagttgaatttgactctggtgactagtatcctaattttatgcagtatggtgcaaggacaagttggagcagatattcataaatcagttaaatacatctatgttgttttaatgaggctaggatactttgatgaaactcctttcaattcattgggcaagaaagatattagcaccaatgagaattttgagttggctgctgaaacaacataagaataagggtgtaaatgaatcaaatcattcgtgaactacttgaggtttggttcgataaaactcgaccaagcttgacttgatttctaaacgagtcaagctcgagcttaccaaacgagccaaacttgaactctatagtattcggctcgttaagactcgtgagctcagctcgttaagaatcgtgagctccgctcatttatgagtttatgagcaggctcgcaaataagctcgtgaacagtctcattaagtaaactgagattattattttaagagaaataaactcaaactctgcatatactttcatgagctaaacttaaannnnnnnnnnnnnnnnnnnnnnnnnnntagttacactttttaacttgcatatattagatcattaagatttaaaaactcttatttataagtttacgaactaatttataaatatacttatttaactaaaattattttaattttaaacttattagttttaaactaaaacttattatacatgtaaataaattgaactactcgtgaactattcgaactaaactcgataaaacttcgactcgtctaagtttgtttgctaaacgagccaaatttgaatttcttaatactaggctcgagttcgaaatgagtaaagttcgaattcgactagaactcaaaaaaatttaatgaaccaagtttgaattcttcaaaactcggctcgacttgattcatttacccctacataagaaggaattgttctttttaaaaatgacaattattttcaattacaagaaacttactctaattggacgacatgacaatactacaaaagcaacaatcagaaattatggaagcttcaattacttaattatcccagaaatataaatatattgtttaagtgttccttgtcgttatgtttcaccaattgaccgatccaaagtaacgtatgaaataggatgtggagatgtagcttgtaaaaatgacggtttaacctttgcactgaattgtaaaaatgacagtaaatataacggtcaacggaaccgagtgcacattcagctgcctgcttgtgggaattgtcccacattggcggattcagaatgggaatcgttccacatcggcggattatggcaattccgaattgtatataatagctagcacgaaattattaaacaacttgggttaaccattttggtccaagtggaaagtggattcaaaagttatttgagccagtttgggccgggctgcttcagtaaaagttcacttatcttatagttttggatgttataaaatagagtactctccttccataaaaagccgaatatgtttataccactcatatgataaggcccaatacgttgatatgatacccccatttggtatggactcacttaggaacttcaattgtcctttttgttgacaacttgaattgtgattaataatctgagatttaggagagagcagagattagagtatgtgtgtgtaaatttagtctgaatagtttaggctatttttctttattttttccctttaatttttttcttttgtcctctgtcaaccgccatcttgttacattgtcgcatgtcgccatcatgcaaaatcatacatatggacgtactgctcctttcttcattgtaaggtgggtatttaataacctccgacgccgcctgacatggttttggatgtaaagaggtttattttccgctgatccatcaagtcaagtgagttagatccttctctattgaacccaagtcctgttctcggctgcctgagcatggcctggactgcgtggtagaggatagatttgcgtattgagttctgatgggattacgctggacttttctacgtgaattcgattgctgctcggatggcaaaaacccagcggttattagtgatcatggaggaacccagtggtcatcaagaaatggaaaaaaattaaatgcaagtaaagcaataattatttactctaagcctccaagatggattattgataacttttacataacaatgaaaagttctcttcctgttataaaataaaaaatatggtaattatgtataattattactttgtttgagtatatataccatattatttattttatattaaattataattaataatttattatgaaatttaaaatataattttattagataatttataaatatataaaaataatttagcaaaaaaaaatttataattattaaatataaaaataatatgatgtaaataaaaaaaatcttaataatcacctaaaagaaaatatagttatggagtacatattagctaccaatttttttgtccaatagctaccaattgtcattttaatttattaaataaataaataaataaattatttcaaagtttatcgattattgattgtatgataaaataaactaaatcatcttaatcttatgtcttatatggatttcaagccggaacaaatttaataaatatgatgtaatgttacttgatttaaatgtttagacaactagattttagtagtgaatataaaattattatctttagtaatttaaaattattaaaaacattatttttattttataatatgataatttacagtgtaattattaaaatttaataaaattcataatttattctctgcttatttaataataaataatttaatctttaattttttattttattaataaaataatttttttataattttatcattattcgattattaattgaataaattaaatcaaattaaaaaattaatttattataaatatttttttacatagtaaggagggtattaaacctttaagcaaagtcacgaaaccatataatttcaacagtatcagcttgcaagtagtaccgagacttcatagaaggcgtttccaaaatttgcatctcaagagagcctttaactgacgatttcgctaagcaatccgccacagcgttagcctcacgaaagatgtgtttgaaataaacttttcatgcataacccaccaaagtcgtaatctcagaaactagatttctaatccggcccgctccatctaaactttattataaaaattaaataattctattgtctttatagataaattgataattttaagaattatttgatataaataatccttattcttttacatatttaaaattgtatttaaattttaaactgatgataattttactactcaaactaatggtagaattatcaaaatatctaatatacaatttatagaaaagtgctaattaattttaatattttaaaattataaatattaaagataatatttgaatcatattatttagaatactgagtttaaatttgaaatttgttttaacttatctggtacaccattattgtaataaaattaaaatgtgaaatttgtttaatgtttttaatatttaattaacttaaatatcaccttatcttgcttctaattgattgtaacaaactttggtatttatcctttttactaaatctttttataagttatgttgtttactattatctatacaataacaaaaatttccttcaaaatcaattcttgttatatcattaattgttttaataaagctacaattttcatgattttctttctaatcttggaaattcaaagaacaaatttccctttatacccttctggtatgagtagtggtctcagatgcttgtaaggctatgaatgacacttgtacgttttaccgtttcatcataaataaatccattaaatacatccactttctacaagaagacaaaacctcgaaacccgacatgaaacggcgtcatttcctctcaaatgaggcaaaaagcatataccaccacggatcccaaacttaacccaagtcaaaactcaacctaatcatgggtaattcctgctccctcaagatttctacacgtgtccgcatccaacggcacccatccaccttgcacagatattcaccgcctcatcttcctatataactactcatcagaccagccaaaaaccctaccctgttctctttatcctctaaagattttccgtctataaatcgacttcctctcttctaatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtgctttgactgctgctgttacggcagcaggaggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgccaacccaacttatgagttctgaggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgatcgagtgtacttcaagtcacaccggcgagtgtttgatcgccggcggtaccgagtgtacttcaagtcactc

SEQ ID NO: 20 - DNA, 22359 bp, artificial
  geminivirus replicon T-DNA vector including a repair template designed to incorporate the cassava rotamase CYP1 annotated promoter into the edited target site to drive T102I/P106A EPSPS expression
  misc_feature (16329)..(16355): n is a, c, g, or t
gtgggaaatcaataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggagaagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcatagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacggctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggcctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggaaacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtagagctcaaagtttaacgcgttagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcggtaccgatcggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgactcgagatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataaggagctccggtgacggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtcagaactcataagttgggtgttttagagctagaaatagcaagttaaaataaggctagtccgttatcaacttgaaaaagtggcaccgagtcggtgctttttttcccggcgccatggatgttgttgttaccagaaagtaaataaatgttcaatctctgatgttctcaagtaagtgagttttattgggaataatattaacttatgttcttcttgcatttgatttctttgccgctctcttcttctatcttaaatctgtgtatactatttcactattgggctttttattagtctataatgggactcaaaataaggctttggcccacatcaaaaagataagtcacaaatcaaaactaaattcagagtcttttctcccacatcggtcactgtactcattttgtgtttgtttatatattacacgaaccgatctttggtacgatatactatacatcaaatgggttttagagctagaaatagcaagttaaaataaggctagtccgttatcaacttgaaaaagtggcaccgagtcggtgctttttttcgcgcgtagtcctcggtaatatcgcagaacaaaagtacctgatggacccacggaattgtgtcagtgaatgctaatgagtgttattgttatctggttgggacggagaaagattgacctatttcaaaagttgtggttgttggaactcgtattctcagccacctatcctatcctcattgtcctctttccacttgccagcattcccaccgccaccaccagtggcactgggatgcggggaaatggatgttcttaattgcttttatttttatttttatttttattttttgaaaaaatcttatgataaaaatatattaggcttgctttttgagttaatttttaataatctacattaatatcttgtacctttttaaataataatttcggcaaaaccagatataataaaatctctcatagaaaatctatttttgtgatacagactcaccaacgaattttttaggcatatccgacgaagtgtaagtggcatattctgtatagggtaatacactgttcattcaaattcgaaaatgatatttttccaataaaatatcacaattcccttaattttgatgatttattatgttcaaattatgctgcggttttccatcctatttccattttatggctgtgtaaatttgattttatccaagaaatccattttaaatcaaatagatacattattaaattttttgttgaattaattaaaatgtatataattagtgtataaatttaatcaattttaaaatgtatatcattaatgtataaattaattaaaataaatttaaatattatgttcattaatcaaattttaattgaagtgattttcccaagcaacttctcgttgtcctaccgtgacgataggctaagcttggtccatcactgtcaaccgaggtgttattatatactatacatcaaatggagcccgaaagtcagaactcataagttgggtgggacatcagtaatagctttgtgtcctggtctaaaggcattgatgggtccctgcaaaagaggatttcattgcgtagtttttgaaagataggcaaagtggtagagatttatatggaataatgactattttttaatccatatacttttttttttatatttgaatccattatatattttgtctttttataatttaatagttgaattgttcaaagttgaatttgactctggtgactagtatcctaattttatgcagtatggtgcaaggacaagttggagcagatattcataaatcagttaaatacatctatgttgttttaatgaggctaggatactttgatgaaactcctttcaattcattgggcaagaaagatattagcaccaatgagaattttgagttggctgctgaaacaacataagaataagggtgtaaatgaatcaaatcattcgtgaactacttgaggtttggttcgataaaactcgaccaagcttgacttgatttctaaacgagtcaagctcgagcttaccaaacgagccaaacttgaactctatagtattcggctcgttaagactcgtgagctcagctcgttaagaatcgtgagctccgctcatttatgagtttatgagcaggctcgcaaataagctcgtgaacagtctcattaagtaaactgagattattattttaagagaaataaactcaaactctgcatatactttcatgagctaaacttaaannnnnnnnnnnnnnnnnnnnnnnnnnntagttacactttttaacttgcatatattagatcattaagatttaaaaactcttatttataagtttacgaactaatttataaatatacttatttaactaaaattattttaattttaaacttattagttttaaactaaaacttattatacatgtaaataaattgaactactcgtgaactattcgaactaaactcgataaaacttcgactcgtctaagtttgtttgctaaacgagccaaatttgaatttcttaatactaggctcgagttcgaaatgagtaaagttcgaattcgactagaactcaaaaaaatttaatgaaccaagtttgaattcttcaaaactcggctcgacttgattcatttacccctacataagaaggaattgttctttttaaaaatgacaattattttcaattacaagaaacttactctaattggacgacatgacaatactacaaaagcaacaatcagaaattatggaagcttcaattacttaattatcccagaaatataaatatattgtttaagtgttccttgtcgttatgtttcaccaattgaccgatccaaagtaacgtatgaaataggatgtggagatgtagcttgtaaaaatgacggtttaacctttgcactgaattgtaaaaatgacagtaaatataacggtcaacggaaccgagtgcacattcagctgcctgcttgtgggaattgtcccacattggcggattcagaatgggaatcgttccacatcggcggattatggcaattccgaattgtatataatagctagcacgaaattattaaacaacttgggttaaccattttggtccaagtggaaagtggattcaaaagttatttgagccagtttgggccgggctgcttcagtaaaagttcacttatcttatagttttggatgttataaaatagagtactctccttccataaaaagccgaatatgtttataccactcatatgataaggcccaatacgttgatatgatacccccatttggtatggactcacttaggaacttcaattgtcctttttgttgacaacttgaattgtgattaataatctgagatttaggagagagcagagattagagtatgtgtgtgtaaatttagtctgaatagtttaggctatttttctttattttttccctttaatttttttcttttgtcctctgtcaaccgccatcttgttacattgtcgcatgtcgccatcatgcaaaatcatacatatggacgtactgctcctttcttcattgtaaggtgggtatttaataacctccgacgccgcctgacatggttttggatgtaaagaggtttattttccgctgatccatcaagtcaagtgagttagatccttctctattgaacccaagtcctgttctcggctgcctgagcatggcctggactgcgtggtagaggatagatttgcgtattgagttctgatgggattacgctggacttttctacgtgaattcgattgctgctcggatggcaaaaacccagcggttattagtgatcatggaggaacccagtggtcatcaagaaatggaaaaaaattaaatgcaagtaaagcaataattatttactctaagcctccaagatggattattgataacttttacataacaatgaaaagttctcttcctgttataaaataaaaaatatggtaattatgtataattattactttgtttgagtatatataccatattatttattttatattaaattataattaataatttattatgaaatttaaaatataattttattagataatttataaatatataaaaataatttagcaaaaaaaaatttataattattaaatataaaaataatatgatgtaaataaaaaaaatcttaataatcacctaaaagaaaatatagttatggagtacatattagctaccaatttttttgtccaatagctaccaattgtcattttaatttattaaataaataaataaataaattatttcaaagtttatcgattattgattgtatgataaaataaactaaatcatcttaatcttatgtcttatatggatttcaagccggaacaaatttaataaatatgatgtaatgttacttgatttaaatgtttagacaactagattttagtagtgaatataaaattattatctttagtaatttaaaattattaaaaacattatttttattttataatatgataatttacagtgtaattattaaaatttaataaaattcataatttattctctgcttatttaataataaataatttaatctttaattttttattttattaataaaataatttttttataattttatcattattcgattattaattgaataaattaaatcaaattaaaaaattaatttattataaatatttttttacatagtaaggagggtattaaacctttaagcaaagtcacgaaaccatataatttcaacagtatcagcttgcaagtagtaccgagacttcatagaaggcgtttccaaaatttgcatctcaagagagcctttaactgacgatttcgctaagcaatccgccacagcgttagcctcacgaaagatgtgtttgaaataaacttttcatgcataacccaccaaagtcgtaatctcagaaactagatttctaatccggcccgctccatctaaactttattataaaaattaaataattctattgtctttatagataaattgataattttaagaattatttgatataaataatccttattcttttacatatttaaaattgtatttaaattttaaactgatgataattttactactcaaactaatggtagaattatcaaaatatctaatatacaatttatagaaaagtgctaattaattttaatattttaaaattataaatattaaagataatatttgaatcatattatttagaatactgagtttaaatttgaaatttgttttaacttatctggtacaccattattgtaataaaattaaaatgtgaaatttgtttaatgtttttaatatttaattaacttaaatatcaccttatcttgcttctaattgattgtaacaaactttggtatttatcctttttactaaatctttttataagttatgttgtttactattatctatacaataacaaaaatttccttcaaaatcaattcttgttatatcattaattgttttaataaagctacaattttcatgattttctttctaatcttggaaattcaaagaacaaatttccctttatacccttctggtatgagtagtggtctcagatgcttgtaaggctatgaatgacacttgtacgttttaccgtttcatcataaataaatccattaaatacatccactttctacaagaagacaaaacctcgaaacccgacatgaaacggcgtcatttcctctcaaatgaggcaaaaagcatataccaccacggatcccaaacttaacccaagtcaaaactcaacctaatcatgggtaattcctgctccctcaagatttctacacgtgtccgcatccaacggcacccatccaccttgcacagatattcaccgcctcatcttcctatataactactcatcagaccagccaaaaaccctaccctgttctctttatcctctaaagattttccgtctataaatcgacttcctctcttctaatggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgaggtacaaatgattatgagtttgtaggcaattgcaatttgtgcccgtttgaattgtagtctgagctaattcatccattttccttttcaattctgaagaaatcaggaaaggggaagctttttctaaaaaaataagactcttacacaattaaattgatcgtcctggttgcattttgcgatgcgagcttaatagaatttgaagaatggagtttcggtagttgaaaataatatatagttggtgcattttatttgagactgaattcctgtttttctgtttacatttatagcttggagggattgtaatggtggattgttcctgtcacgattatgattcgagtttgttatgtattcgtacctggacatcatattattagctgctcgataaactaagggggcaaaaattatttttatggtgcttataaatttttgtatgtagtgtctaaagagaatgtatgagctggagtaatttgacgcaaaaatttaaaaaagaagcaaatttgactttagaacctctaactggattttactttctaatgtttatccacttgtttccaggaagattttctcagagttgcttatgttattgaagaataagtggcttcaatttttggcttttctaacttcagttcaaacaaatgagagtcccttgagtgatcattgattgtctttgtgactttgtctatcattcagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaattgcaatgcgtgctttgactgctgctgttacggcagcaggaggaaattcaaggtctcccttatcttgctttcctttacttcctgttttatcttgctcctgttacaccctgttaatgttgccttttaacaaattaatgtgggaaaattcaactcctaccttgcttcgagtattaatggaattgctcgccaacccaacttatgagttctgaggccaactatatgcgaagttgcaaactgaagttgtatgtggccctaatgtctatcaccaagctttctaatctctctctgtgagccctatttggcatggtctaaaattgacaaggatggatttccccaccccatgatacctgcatctccatcttgatgggatgcagacctgacacctccaggttagaagtttgaaacataaataaattgtctcgctgggacaatatgaatattattttggcagaggttccatcttatatgatattctctgtgacacgtttggagtaagtgtcaatttaggattctcataggtggcgagcccagtaggacattttttaaaacttctccaacctgattgtacgtgtgaacattgtttggatccttggcccactgtttaattatattttttccattgaatgcaacaatcaattgacacccgcttttacatagtaggattctttctatatggtgtcatctgcctcctgcgccctttagtttcaatgtatccttttctcttcctgtctatttgatttaactattcaaaatatttcagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggtctgattgctatcacttatacattaatattttgttgaacatgtttgataacttgtcatgcctgtatcttctggcaatatgtaatgctgcaatctctattataataattagaaggtagctttaagttctgtatcagcatgctgtggatgatcgagtgtacttcaagtcacaccggcgagtgtttgatcgccggcggtaccgagtgtacttcaagtca

SEQ ID NO: 21 - DNA, 1173 bp, Zea mays
atgctcggggccttgaggactcttggtctctctgtcgaagcggacaaagctgccaaaagagctgtagttgttggctgtggtggaaagttcccagttgaggattctaaagaggaagtgcagctcttcttggggaatgctggaactgcaatgcggccattgacagcagctgttactgctgctggtggaaatgcaacttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggtcaagctgtctggctccatcagcagtcagtacttgagtgccttgctgatggctgctcctttggctcttggggatgtggagattgaaatcattgataaattaatctccattccctacgtcgaaatgacattgagattgatggagcgttttggtgtgaaagcagagcattctgatagctgggacagattctacattaagggaggtcaaaaatacaagtcccctaaaaatgcctatgttgaaggtgatgcctcaagcgcaagctatttcttggctggtgctgcaattactggagggactgtgactgtggaaggttgtggcaccaccagtttgcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacgtggcttcctggagagtaaaggagaccgagaggatggttgcgatccggacggagctaaccaagctgggagcatctgttgaggaagggccggactactgcatcatcacgccgccggagaagctgaacgtgacggcgatcgacacgtacgacgaccacaggatggccatggccttctcccttgccgcctgtgccgaggtccccgtgaccatccgggaccctgggtgcacccggaagaccttccccgactacttcgatgtgctgagcactttcgtcaagaattaa

SEQ ID NO: 22 - protein, 390 aa, Zea mays
  MISC_FEATURE (47)..(47): the glycine can be substituted with, for example, alanine
  MISC_FEATURE (48)..(48): the threonine can be substituted with, for example, isoleucine
  MISC_FEATURE (51)..(51): the proline can be substituted with, for example, alanine, serine, threonine, glycine, cysteine, isoleucine, valine, methionine, or leucine
  MISC_FEATURE (90)..(90): the glycine can be substituted with, for example, asparagine or aspartic acid
  MISC_FEATURE (135)..(135): the alanine can be substituted with, for example, threonine
MLGALRTLGLSVEADKAAKRAVVVGCGGKFPVEDSKEEVQLFLGNAGTAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVTIRDPGCTRKTFPDYFDVLSTFVKN

SEQ ID NO: 23 - DNA, 1569 bp, Manihot esculenta
atggcccaagtgagcaaaatctgcaatggggctctaaataattgtagtctccccaatctctcgaaaccccaaagacccaaatacttatcttcaatttcatttagatcacaacttcaggggtcttcactttcatgggctttaaagcagtgtcaaaggaaggctgactatgaagttggtacagttaagatgatccctgttagagtttcagcttcagtagccgcagcggagaagccttcaacagcaccagagatagtcttgcaacccattaaagaaatctccggtaccgtcaacttgccgggttccaagtctctatccaatcggattctccttctcgcagctctttctgagggtataactgttgttgacaacttgctgaacagtgatgatgttcgttacatgcttggtgcactgagaacgcttgggttgcatgtggaagacaatagtgaactcaaacaagccattgtggaaggttgtggcggtcattttccagtgggtaaagaatcaaaagatgaccttgaacttttccttggaaatgcaggaacagcaatgcgtccattgactgctgctgttacggcagcaggtggaaattcaagctacatacttgatggggtcccacgaatgcgagagagaccaattggagatttggttgctggtcttaagcagcttggtgcagatgttgaatgttctcctactaactgccccccagttcgtgtaaatggaaagggaggccttcctggggggaaggttaagctctctggttcaattagcagtcaatacttgactgctttgctcatggcagctcctttggctcttggagatgtagaaattgagattattgataaattgatatccattccttatgttgatatgactttgaagttaatggaacgattcggagtcactgcagaacacagtggtagctgggatcgtttcttcattcgtgggggtcaaaagtacaaatctcctggaaaagcttttgttgaaggtgatgcttcgagtgccagttatttcctggctggtgcagcaatcactggtggaaccatcactgttgaaggttgtggaacaagtagtttgcagggggatgtaaggtttgctgaagtgcttgacaaaatgggagcaaaagtcacctggacagagaacagtgttactgtgactggaccaccacgcaattctcctattaaaaagcacttgcgagctattgatgtcaacatgaacaaaatgccagatgttgctatgacattggccgtggttgcactttttgctgatggccctactgccataagagacgtggccagttggagagttaaagaaacagaacggatgattgcaatttgcacagagctcaggaagctcggagcaaatgttgaagagggaccagactactgtttgatcactccacctgagaaactgaacatcacagagattgacacttatgatgatcacagaatggccatggcgttctcccttgctgcctgcggggatgttccagtcacaatcaaggaccctggttgcacaagaaaaacttttccagactactttgaggtccttgagaggttcactaagcattga

SEQ ID NO: 24 - protein, 522 aa, Manihot esculenta
  MISC_FEATURE (180)..(180): the glycine can be substituted with, for example, alanine
  MISC_FEATURE (181)..(181): the threonine can be substituted with, for example, isoleucine
  MISC_FEATURE (185)..(185): the proline can be substituted with, for example, alanine, serine, threonine, glycine, cysteine, isoleucine, valine, methionine, or leucine
  MISC_FEATURE (223)..(223): the glycine can be substituted with, for example, asparagine or aspartic acid
  MISC_FEATURE (267)..(267): the alanine can be substituted with, for example, threonine
MAQVSKICNGALNNCSLPNLSKPQRPKYLSSISFRSQLQGSSLSWALKQCQRKADYEVGTVKMIPVRVSASVAAAEKPSTAPEIVLQPIKEISGTVNLPGSKSLSNRILLLAALSEGITVVDNLLNSDDVRYMLGALRTLGLHVEDNSELKQAIVEGCGGHFPVGKESKDDLELFLGNAGTAMRPLTAAVTAAGGNSSYILDGVPRMRERPIGDLVAGLKQLGADVECSPTNCPPVRVNGKGGLPGGKVKLSGSISSQYLTALLMAAPLALGDVEIEIIDKLISIPYVDMTLKLMERFGVTAEHSGSWDRFFIRGGQKYKSPGKAFVEGDASSASYFLAGAAITGGTITVEGCGTSSLQGDVRFAEVLDKMGAKVTWTENSVTVTGPPRNSPIKKHLRAIDVNMNKMPDVAMTLAVVALFADGPTAIRDVASWRVKETERMIAICTELRKLGANVEEGPDYCLITPPEKLNITEIDTYDDHRMAMAFSLAACGDVPVTIKDPGCTRKTFPDYFEVLERFTKH

SEQ ID NO: 25 - DNA, 1548 bp, Oryza sativa
atggcggcgaccatggcgtccaacgccgcggctgcggcggcggtgtccctggaccaggccgtggcggcgtcggcggcgttctcgtcgcggaagcagctgcggctgcccgccgcggcgcgcggggggatgcgggtgcgggtgcgggcgcgggggcggcgggaggcggtggtggtggcgtccgcgtcgtcgtcgtcggtggcagcgccggcggcgaaggcggaggagatcgtgctccagcccatcagggagatctccggggcggttcagctgccagggtccaagtcgctctccaacaggatcctcctcctctccgccctctccgagggcacaacagtggtggacaacttgctgaacagtgaggatgttcactacatgcttgaggccctgaaagccctcgggctctctgtggaagcagataaagttgcaaaaagagctgtagtcgttggctgtggtggcaagtttcctgttgagaaggatgcgaaagaggaagtgcaactcttcttggggaacgctggaactgcaatgcgaccattgacagcagccgtgactgctgctggtggaaatgcaacttatgtgcttgatggagtgccacgaatgagggagagaccgattggtgacttggttgtcgggttgaaacaacttggtgcggatgtcgactgtttccttggcactgaatgcccacctgttcgtgtcaagggaattggaggacttcctggtggcaaggttaagctctctggttccatcagcagtcagtacttgagtgccttgctgatggctgctcctttggcccttggggatgtggagatcgaaatcattgacaaactaatctccattccttacgttgaaatgacattgagattgatggagcgttttggtgtgaaggcagagcattctgatagttgggacagattctatattaagggagggcagaagtacaaatctcctggaaatgcctatgttgaaggtgatgcctcaagcgcgagctatttcttggctggtgctgcaatcactggaggcactgtgacagttcaaggttgtggtacgaccagtttgcagggtgatgtcaaatttgctgaggtacttgagatgatgggagcaaaggttacatggactgacaccagtgtaaccgtaactggtccaccacgtgagccttatgggaagaaacacctgaaagctgttgatgtcaacatgaacaaaatgcctgatgttgccatgacccttgccgttgttgcactcttcgctgatggtccaactgctatcagagatgtggcttcctggagagtaaaggaaaccgaaaggatggttgcaattcggaccgagctaacaaagctgggagcatcggttgaagaaggtcctgactactgcatcatcaccccaccggagaagctgaacatcacggcaatcgacacctacgatgatcacaggatggccatggccttctccctcgctgcctgcgccgacgtgcccgtgacgatcagggaccctggttgcacccgcaagaccttccccaactacttcgacgttctaagcactttcgtcaggaactga

SEQ ID NO: 26 - protein, 515 aa, Oryza sativa
  MISC_FEATURE (172)..(172): the glycine can be substituted with, for example, alanine
  MISC_FEATURE (173)..(173): the threonine can be substituted with, for example, isoleucine
  MISC_FEATURE (177)..(177): the proline can be substituted with, for example, alanine, serine, threonine, glycine, cysteine, isoleucine, valine, methionine, or leucine
  MISC_FEATURE (215)..(215): the glycine can be substituted with, for example, asparagine or aspartic acid
  MISC_FEATURE (260)..(260): the alanine can be substituted with, for example, threonine
MAATMASNAAAAAAVSLDQAVAASAAFSSRKQLRLPAAARGGMRVRVRARGRREAVVVASASSSSVAAPAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAGTAMRPLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDVAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 27 - DNA, 1563 bp, Arabidopsis thaliana
atggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcggagaaagcgtcggagattgtacttcaacccattagagaaatctccggtcttattaagcttcctggctccaagtctctatcaaatcggatcctgcttctcgctgctctgtctgagggaacaactgtagtggacaacttgttgaatagcgatgacatcaattacatgcttgatgcgttgaagagattgggacttaatgtggaaactgacagtgaaaataatcgtgctgtagttgaaggatgtggcgggatattcccagcttccatagattcaaagagtgatatcgaactttacctcggtaatgcaggaacagcaatgcgtccacttaccgctgcggtcactgctgcaggtggaaacgcaagttatgtgcttgatggggtgcctcgtatgagagaaagacctataggggatttggttgttggtcttaagcagcttggtgctgatgttgaatgtactcttggaactaactgccctcctgttcgtgtcaacgctaatggtggccttcccggtggaaaggtgaagctttctggatcaattagtagtcagtacttgactgctctgctcatgtctgctcccttagctcttggagacgtcgagattgagattgtcgataaattaatttctgttccatatgttgaaatgacattgaagttgatggaacgtttcggggttagtgtcgagcatagtgatagctgggatcgtttctttgtcaagggcgggcaaaaatacaagtctccgggtaatgcgtatgtagaaggtgatgcttctagtgctagttatttcttggctggtgctgccattaccggtgaaactgtcacagtcgaaggttgtggaactaccagcttgcagggagatgtaaaattcgccgaggtccttgagaaaatgggatgtaaagtgtcctggacagagaacagtgtgactgtgacaggaccacctagagatgcttttggaatgagacacttgcgggctattgatgtcaacatgaacaaaatgcctgatgtagccatgacccttgccgtcgttgctctctttgctgacggtccaaccaccattagagatgtggctagctggagagtaaaggagacagaaaggatgattgccatttgcacagagcttagaaaactgggagctacagtggaagaaggttcagattattgtgtgataactccgcccaaaaaggtgaaaacggcagagattgatacatatgatgatcatagaatggcaatggcattctctcttgcagcttgtgctgatgttccaatcaccatcaacgatcctggttgcaccaggaaaaccttccccgactacttccaagtacttgaaagaatcacaaagcactaa

SEQ ID NO: 28 - protein, 520 aa, Arabidopsis thaliana
  MISC_FEATURE (177)..(177): the glycine can be substituted with, for example, alanine
  MISC_FEATURE (178)..(178): the threonine can be substituted with, for example, isoleucine
  MISC_FEATURE (182)..(182): the proline can be substituted with, for example, alanine, serine, threonine, glycine, cysteine, isoleucine, valine, methionine, or leucine
  MISC_FEATURE (220)..(220): the glycine can be substituted with, for example, asparagine or aspartic acid
  MISC_FEATURE (265)..(265): the alanine can be substituted with, for example, threonine
MAQVSRICNGVQNPSLISNLSKSSQRKSPLSVSLKTQQHPRAYPISSSWGLKKSGMTLIGSELRPLKVMSSVSTAEKASEIVLQPIREISGLIKLPGSKSLSNRILLLAALSEGTTVVDNLLNSDDINYMLDALKRLGLNVETDSENNRAVVEGCGGIFPASIDSKSDIELYLGNAGTAMRPLTAAVTAAGGNASYVLDGVPRMRERPIGDLVVGLKQLGADVECTLGTNCPPVRVNANGGLPGGKVKLSGSISSQYLTALLMSAPLALGDVEIEIVDKLISVPYVEMTLKLMERFGVSVEHSDSWDRFFVKGGQKYKSPGNAYVEGDASSASYFLAGAAITGETVTVEGCGTTSLQGDVKFAEVLEKMGCKVSWTENSVTVTGPPRDAFGMRHLRAIDVNMNKMPDVAMTLAVVALFADGPTTIRDVASWRVKETERMIAICTELRKLGATVEEGSDYCVITPPKKVKTAEIDTYDDHRMAMAFSLAACADVPITINDPGCTRKTFPDYFQVLERITKH

SEQ ID NO: 29 - DNA, 1551 bp, Petunia hybrid
atggcacaaattaacaacatggctcaagggatacaaacccttaatcccaattccaatttccataaaccccaagttcctaaatcttcaagttttcttgtttttggatctaaaaaactgaaaaattcagcaaattctatgttggttttgaaaaaagattcaatttttatgcaaaagttttgttcctttaggatttcagcatcagtggctacagcacagaagccttctgagatagtgttgcaacccattaaagagatttcaggcactgttaaattgcctggctctaaatcattatctaatagaattctccttcttgctgccttatctgaaggaacaactgtggttgacaatttactaagtagtgatgatattcattacatgcttggtgccttgaaaacacttggactgcatgtagaagaagatagtgcaaaccaacgagctgttgttgaaggttgtggtgggcttttccctgttggtaaagagtccaaggaagaaattcaactgttccttggaaatgcaggaacagcaatgcggccactaacagcagcagttactgtagctggtggaaattcaaggtatgtacttgatggagttcctcgaatgagagagagaccaattagtgatttggttgatggtcttaaacagcttggtgcagaggttgattgtttccttggtacgaaatgtcctcctgttcgaattgtcagcaagggaggtcttcctggagggaaggtcaagctctctggatccattagcagccaatacttgactgctctgcttatggctgctccactggctttaggagatgtggagattgaaatcattgacaaactaattagtgtaccttatgtcgagatgacattgaagttgatggagcgatttggtatttctgtggagcacagtagtagctgggacaggttctttgtccgaggaggtcagaaatacaagtctcctggaaaagcttttgtcgaaggtgatgcttcaagtgctagctacttcttggctggtgcagcagtcacaggtggaactatcactgttgaaggttgtgggacaaacagtttacagggggatgtcaaatttgctgaggtacttgaaaaaatgggagctgaagttacgtggacagagaacagtgtcacagtcaaaggacctccaaggagttcttctgggaggaagcatttgcgtgccattgatgtgaacatgaataaaatgcctgatgttgccatgacacttgctgttgttgcactttatgctgatggtcccacagctataagagatgttgctagctggagagtcaaggaaactgagcgcatgatcgccatatgcacagaacttaggaagttaggagcaaccgttgaagaaggaccagactactgcataatcaccccaccggagaaactaaatgtgaccgatattgatacatacgatgatcacaggatggccatggctttttctcttgctgcttgtgcagatgttcccgtcaccatcaatgaccctggctgcacgcggaaaaccttccctaactactttgatgtacttcagcagtactccaagcattga

SEQ ID NO: 30 - protein, 516 aa, Petunia hybrid
  MISC_FEATURE (173)..(173): the glycine can be substituted with, for example, alanine
  MISC_FEATURE (174)..(174): the threonine can be substituted with, for example, isoleucine
  MISC_FEATURE (178)..(178): the proline can be substituted with, for example, alanine, serine, threonine, glycine, cysteine, isoleucine, valine, methionine, or leucine
  MISC_FEATURE (216)..(216): the glycine can be substituted with, for example, asparagine or aspartic acid
  MISC_FEATURE (271)..(271): the alanine can be substituted with, for example, threonine
MAQINNMAQGIQTLNPNSNFHKPQVPKSSSFLVFGSKKLKNSANSMLVLKKDSIFMQKFCSFRISASVATAQKPSEIVLQPIKEISGTVKLPGSKSLSNRILLLAALSEGTTVVDNLLSSDDIHYMLGALKTLGLHVEEDSANQRAVVEGCGGLFPVGKESKEEIQLFLGNAGTAMRPLTAAVTVAGGNSRYVLDGVPRMRERPISDLVDGLKQLGAEVDCFLGTKCPPVRIVSKGGLPGGKVKLSGSISSQYLTALLMAAPLALGDVEIEIIDKLISVPYVEMTLKLMERFGISVEHSSSWDRFFVRGGQKYKSPGKAFVEGDASSASYFLAGAAVTGGTITVEGCGTNSLQGDVKFAEVLEKMGAEVTWTENSVTVKGPPRSSSGRKHLRAIDVNMNKMPDVAMTLAVVALYADGPTAIRDVASWRVKETERMIAICTELRKLGATVEEGPDYCIITPPEKLNVTDIDTYDDHRMAMAFSLAACADVPVTINDPGCTRKTFPNYFDVLQQYSKH

SEQ ID NO: 31 - DNA, 94 bp, artificial
  expected gene targeting product of glyphosate tolerant plant generated by NHEJ using vector H055
gggtaaagaatcaaaagatgaccttgaacttttccttggaaatgcaggaattgcaatgcgtgctttgactgctgctgttacggcagcaggagga

SEQ ID NO: 32 - DNA, 45 bp, artificial
  sequencing read to verify gene editing events
  misc_feature (40)..(40): n is a, c, g, or t
gggtaaagaatcaaaagatgaccttgaacttttccttggnaatgc

SEQ ID NO: 33 - DNA, 94 bp, artificial; identical to SEQ ID NO: 31
  sequencing read to verify gene editing events

SEQ ID NO: 34 - DNA, 94 bp, artificial; identical to SEQ ID NO: 31
  consensus sequence of sequencing reads to verify gene editing events

SEQ ID NO: 35 - DNA, 78 bp, artificial
  expected gene targeting product of glyphosate tolerant plant generated by NHEJ using vector H055
cttgcttcgagtattaatggaattgctcgccaaccggttggggccaactatatgcgaagttgcaaactgaagttgtat

SEQ ID NO: 36 - DNA, 90 bp, artificial
  sequencing read to verify gene editing events
cttgcttcgagtattaatggaattgctcgccaaccacaacttatgagttctgaggccaactatatgcgaagttgcaaactgaagttgtat

SEQ ID NO: 37 - DNA, 90 bp, artificial; identical to SEQ ID NO: 36
  sequencing read to verify gene editing events

SEQ ID NO: 38 - DNA, 68 bp, artificial
  sequencing read to verify gene editing events
  misc_feature (10)..(10): n is a, c, g, or t
tgctcgccanccacaaccttatgagttctgaggccaactatatgcgaagttgcaaactgaagttgtat

SEQ ID NO: 39 - DNA, 90 bp, artificial; identical to SEQ ID NO: 36
  consensus sequence of sequencing reads to verify gene editing events

SEQ ID NO: 40 - DNA, 94 bp, artificial; identical to SEQ ID NO: 31
  expected gene targeting product of glyphosate tolerant plant generated by NHEJ using vector H056

SEQ ID NO: 41 - DNA, 94 bp, artificial
  sequencing read to verify gene editing events
  misc_feature (40)..(40): n is a, c, g, or t
gggtaaagaatcaaaagatgaccttgaacttttccttggnaatgcaggaattgcaatgcgtgctttgactgctgctgttacggcagcaggagga

SEQ ID NO: 42 - DNA, 94 bp, artificial; identical to SEQ ID NO: 31
  sequencing read to verify gene editing events

SEQ ID NO: 43 - DNA, 48 bp, artificial
  sequencing read to verify gene editing events
  misc_feature (1)..(6): n is a, c, g, or t
nnnnnntgcatgcgtgctttgactgctgctgttacggcagcaggagga

SEQ ID NO: 44 - DNA, 94 bp, artificial; identical to SEQ ID NO: 31
  consensus sequence of sequencing reads to verify gene editing events

SEQ ID NO: 45 - DNA, 82 bp, artificial
  expected gene targeting product of glyphosate tolerant plant generated by NHEJ using vector H056
aatggaattgctcgttcagaactcataagttgttggggccaactatatgcgaagttgcaaactgaagttgtatgtggcccta

SEQ ID NO: 46 - DNA, 82 bp, artificial; identical to SEQ ID NO: 45
  sequencing read to verify gene editing events

SEQ ID NO: 47 - DNA, 82 bp, artificial; identical to SEQ ID NO: 45
  sequencing read to verify gene editing events

SEQ ID NO: 48 - DNA, 82 bp, artificial; identical to SEQ ID NO: 45
  sequencing read to verify gene editing events

SEQ ID NO: 49 - DNA, 82 bp, artificial; identical to SEQ ID NO: 45
  consensus sequence of sequencing reads to verify gene editing events

SEQ ID NO: 50 - DNA, 25 bp, artificial
  synthetic oligonucleotide primer
gcttcgagtgccagttatttcctgg

SEQ ID NO: 51 - DNA, 25 bp, artificial
  synthetic oligonucleotide primer
caagcacttcagcaaaccttacatc

SEQ ID NO: 52 - DNA, 21 bp, artificial
  synthetic oligonucleotide primer
gttgccttcttttgcgtttct

SEQ ID NO: 53 - DNA, 21 bp, artificial
  synthetic oligonucleotide primer
gcaatttgatccgttttccat

SEQ ID NO: 54 - DNA, 23 bp, artificial
  synthetic oligonucleotide primer
ttgcaatttgcacagagctcagg

SEQ ID NO: 55 - DNA, 20 bp, artificial
  synthetic oligonucleotide primer
tcgtggtccttgtagtcgcc